SEQUENCE LISTING (39 sequences)

SEQ ID NO: 1 - DNA, 7561 bp, VEE virus
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 2 - RNA, 7561 nt, VEE virus
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaag

SEQ ID NO: 3 - DNA, 66 bp, VEE virus
tctagaggcccctataactctctacggctaacctgaatggactacgacatagtctagtccgccaag

SEQ ID NO: 4 - RNA, 66 nt, VEE virus
ucuagaggccccuauaacucucuacggcuaaccugaauggacuacgacauagucuaguccgccaag

SEQ ID NO: 5 - DNA, 2439 bp, VEE virus
tctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 6 - RNA, 2439 nt, VEE virus
ucuagacggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaagaagagcguuuaaacacgugauaucuggccucaugggccuuccuuucacugcccgcuuuccagucgggaaaccugucgugccagcugcauuaacauggucauagcuguuuccuugcguauugggcgcucuccgcuuccucgcucacugacucgcugcgcucggucguucggguaaagccuggggugccuaaugagcaaaaggccagcaaaaggccaggaaccguaaaaaggccgcguugcuggcguuuuuccauaggcuccgccccccugacgagcaucacaaaaaucgacgcucaagucagagguggcgaaacccgacaggacuauaaagauaccaggcguuucccccuggaagcucccucgugcgcucuccuguuccgacccugccgcuuaccggauaccuguccgccuuucucccuucgggaagcguggcgcuuucucauagcucacgcuguagguaucucaguucgguguaggucguucgcuccaagcugggcugugugcacgaaccccccguucagcccgaccgcugcgccuuauccgguaacuaucgucuugaguccaacccgguaagacacgacuuaucgccacuggcagcagccacugguaacaggauuagcagagcgagguauguaggcggugcuacagaguucuugaagugguggccuaacuacggcuacacuagaagaacaguauuugguaucugcgcucugcugaagccaguuaccuucggaaaaagaguugguagcucuugauccggcaaacaaaccaccgcugguagcggugguuuuuuuguuugcaagcagcagauuacgcgcagaaaaaaaggaucucaagaagauccuuugaucuuuucuacggggucugacgcucaguggaacgaaaacucacguuaagggauuuuggucaugaauacacggugccugacugcguuagcaauuuaacugugauaaacuaccgcauuaaagcuuaucgaugauaagcugucaaacaugagaauucuuagaaaaacucaucgagcaucaaaugaaacugcaauuuauucauaucaggauuaucaauaccauauuuuugaaaaagccguuucuguaaugaaggagaaaacucaccgaggcaguuccauaggauggcaagauccugguaucggucugcgauuccgacucguccaacaucaauacaaccuauuaauuuccccucgucaaaaauaagguuaucaagugagaaaucaccaugagugacgacugaauccggugagaauggcaaaagcuuaugcauuucuuuccagacuuguucaacaggccagccauuacgcucgucaucaaaaucacucgcaucaaccaaaccguuauucauucgugauugcgccugagcgagacgaaauacgcgaucgcuguuaaaaggacaauuacaaacaggaaucgaaugcaaccggcgcaggaacacugccagcgcaucaacaauauuuucaccugaaucaggauauucuucuaauaccuggaaugcuguuuucccggggaucgcaguggugaguaaccaugcaucaucaggaguacggauaaaaugcuugauggucggaagaggcauaaauuccgucagccaguuuagucugaccaucucaucuguaacaucauuggcaacgcuaccuuugccauguuucagaaacaacucuggcgcaucgggcuucccauacaaucgauagauugucgcaccugauugcccgacauuaucgcgagcccauuuauacccauauaaaucagcauccauguuggaauuuaaucgcggccucgagcaagacguuucccguugaauauggcucauaacaccccuuguauuacuguuuauguaagcagacaguuuuauuguucaugagcggauacauauuugaauguauuuagaaaaauaaacaaauagggguuccgcgcacauuuccccgaaaagugccaccuaaauuguaagcguuaauauuuuguuaaaauucgcguuaaauuuuuguuaaaucagcucauuuuuuaaccaauaggccgaaaucggcaaaaucccuuauaaaucaaaagaauagaccgagauaggguugaguggccgcuacagggcgcucccauucgccauucaggcugcgcaacuguugggaagggcguuucggugcgggccucuucgcuauuacgccagcuggcgaaagggggaugugcugcaaggcgauuaaguuggguaacgccaggguuuucccagucacacgcguaauacgacucacuauag

SEQ ID NO: 7 - DNA, 7561 bp, VEE virus
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagattcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtatgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 8 - RNA, 7561 nt, VEE virus
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagauucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacugguaugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaag

SEQ ID NO: 9 - DNA, 723 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataa

SEQ ID NO: 10 - RNA, 723 nt, Aquaia victoria
auggugagcaagggcgaggagcuguucaccgggguggugcccauccuggucgagcuggacggcgacguaaacggccacaaguucagcguguccggcgagggcgagggcgaugccaccuacggcaagcugacccugaaguucaucugcaccaccggcaagcugcccgugcccuggcccacccucgugaccacccugaccuacggcgugcagugcuucagccgcuaccccgaccacaugaagcagcacgacuucuucaaguccgccaugcccgaaggcuacguccaggagcgcaccaucuucuucaaggacgacggcaacuacaagacccgcgccgaggugaaguucgagggcgacacccuggugaaccgcaucgagcugaagggcaucgacuucaaggaggacggcaacauccuggggcacaagcuggaguacaacuacaacagccacaacgucuauaucauggccgacaagcagaagaacggcaucaaggugaacuucaagauccgccacaacaucgaggacggcagcgugcagcucgccgaccacuaccagcagaacacccccaucggcgacggccccgugcugcugcccgacaaccacuaccugagcacccaguccgcccugagcaaagaccccaacgagaagcgcgaucacaugguccugcuggaguucgugaccgccgccgggaucacucucggcauggacgagcuguacaagugauaa

SEQ ID NO: 11 - protein, 239 aa, Aquaia victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 12 - DNA, 723 bp, Aquaia victoria; identical to SEQ ID NO: 9

SEQ ID NO: 13 - RNA, 723 nt, Aquia victoria; identical to SEQ ID NO: 10

SEQ ID NO: 14 - protein, 239 aa, Aquaia victoria; identical to SEQ ID NO: 11

SEQ ID NO: 15 - DNA, 696 bp, Influenza A virus
atggaccctaatacggtctcttctttccaagtggattgctttctgtggcatgttagaaagagagtggctgatcaagaattgggtgatgcaccgttcttggatagactgcgacgcgatcagaagtcccttcgcggtagaggcagcacgcttgggttggacatcaaaactgcgactcgagccgggaaacaaattgtagagaggattcttaaggaggaaagtgacgaggcactcaaaatgacaatggcaagcgtaccggccagtcggtatttgacagacatgactctcgaagagatgtcacgagattggtccatgctcataccgaagcagaaggtggccggtcctctctgtattagaatggatcaggcgattatggataagaatatcattttgaaggccaatttttctgtcattttcgacagactggaaacattgattctgctccgggctttcaccgaggaaggtgcaatcgtaggtgagatcagtccgctccctagccttcccggtcataccgcagaggatgttaaaaacgctgtgggtgttttgattgggggcttggagtggaacgacaacacagtcagggtgagcgagacacttcaacggtttgcatggcggagtagtaacgagaacggacgcccgcccctcacaccaaagcagaaacgagagatggcaggaactatccgatctgaggtctgataa

SEQ ID NO: 16 - RNA, 696 nt, Influenza A virus
auggacccuaauacggucucuucuuuccaaguggauugcuuucuguggcauguuagaaagagaguggcugaucaagaauugggugaugcaccguucuuggauagacugcgacgcgaucagaagucccuucgcgguagaggcagcacgcuuggguuggacaucaaaacugcgacucgagccgggaaacaaauuguagagaggauucuuaaggaggaaagugacgaggcacucaaaaugacaauggcaagcguaccggccagucgguauuugacagacaugacucucgaagagaugucacgagauugguccaugcucauaccgaagcagaagguggccgguccucucuguauuagaauggaucaggcgauuauggauaagaauaucauuuugaaggccaauuuuucugucauuuucgacagacuggaaacauugauucugcuccgggcuuucaccgaggaaggugcaaucguaggugagaucaguccgcucccuagccuucccggucauaccgcagaggauguuaaaaacgcuguggguguuuugauugggggcuuggaguggaacgacaacacagucagggugagcgagacacuucaacgguuugcauggcggaguaguaacgagaacggacgcccgccccucacaccaaagcagaaacgagagauggcaggaacuauccgaucugaggucugauaa

SEQ ID NO: 17 - protein, 230 aa, Influenza A virus
MDPNTVSSFQVDCFLWHVRKRVADQELGDAPFLDRLRRDQKSLRGRGSTLGLDIKTATRAGKQIVERILKEESDEALKMTMASVPASRYLTDMTLEEMSRDWSMLIPKQKVAGPLCIRMDQAIMDKNIILKANFSVIFDRLETLILLRAFTEEGAIVGEISPLPSLPGHTAEDVKNAVGVLIGGLEWNDNTVRVSETLQRFAWRSSNENGRPPLTPKQKREMAGTIRSEV

SEQ ID NO: 18 - DNA, 276 bp, Influenza 1918 strain
atgggacaagagcaggacacaccgtggattctgtctaccggacatataagtacgcaaaagcgcgaggatgggcagcagactccccggctcgagcaccacaatagtacacggcttatggaccattgccagaagacaatgaaccaggtagtaatgccaaagcaaatcgtctattggaaacaatggctctctctgaggagtcccacacctgtgtccctgaagacccgcgtcttgaaaaggtggcgcttgttctctaaacatgagtggacatcttgataa

SEQ ID NO: 19 - RNA, 276 nt, Influenza 1918 strain
augggacaagagcaggacacaccguggauucugucuaccggacauauaaguacgcaaaagcgcgaggaugggcagcagacuccccggcucgagcaccacaauaguacacggcuuauggaccauugccagaagacaaugaaccagguaguaaugccaaagcaaaucgucuauuggaaacaauggcucucucugaggagucccacaccugugucccugaagacccgcgucuugaaaagguggcgcuuguucucuaaacaugaguggacaucuugauaa

SEQ ID NO: 20 - protein, 90 aa, Influenza 1918 strain
MGQEQDTPWILSTGHISTQKREDGQQTPRLEHHNSTRLMDHCQKTMNQVVMPKQIVYWKQWLSLRSPTPVSLKTRVLKRWRLFSKHEWTS

SEQ ID NO: 21 - DNA, 576 bp, Vaccinia virus
atgagtaaaatatatattgacgaacggagtaacgctgagatcgtttgtgaggctattaagacaatagggatcgaaggagcgacggcggcgcaactcacccgccagttgaatatggaaaaacgagaagtcaataaggcgctgtatgacctccagcgatcagccatggtttactctagtgatgatattccaccaagatggttcatgacgaccgaggctgataaacctgatgcagatgcaatggccgacgtcattattgacgatgtgagccgcgagaagtccatgcgcgaggatcataaatcttttgatgatgtcatcccggccaaaaaaataatcgactggaaaggagcgaatcccgtgaccgtcataaacgagtattgccagataacacgacgagattggagcttcaggattgagtccgttggtccgtctaactctcccaccttctatgcgtgtgttgacatagacggtagggtctttgacaaagctgatggtaaatccaagcgagacgccaagaataatgcagccaaactcgccgtagataagttgctgggatacgtgattatccgattctaataa

SEQ ID NO: 22 - RNA, 576 nt, Vaccinia virus
augaguaaaauauauauugacgaacggaguaacgcugagaucguuugugaggcuauuaagacaauagggaucgaaggagcgacggcggcgcaacucacccgccaguugaauauggaaaaacgagaagucaauaaggcgcuguaugaccuccagcgaucagccaugguuuacucuagugaugauauuccaccaagaugguucaugacgaccgaggcugauaaaccugaugcagaugcaauggccgacgucauuauugacgaugugagccgcgagaaguccaugcgcgaggaucauaaaucuuuugaugaugucaucccggccaaaaaaauaaucgacuggaaaggagcgaaucccgugaccgucauaaacgaguauugccagauaacacgacgagauuggagcuucaggauugaguccguugguccgucuaacucucccaccuucuaugcguguguugacauagacgguagggucuuugacaaagcugaugguaaauccaagcgagacgccaagaauaaugcagccaaacucgccguagauaaguugcugggauacgugauuauccgauucuaauaa

SEQ ID NO: 23 - protein, 190 aa, Vaccinia
MSKIYIDERSNAEIVCEAIKTIGIEGATAAQLTRQLNMEKREVNKALYDLQRSAMVYSSDDIPPRWFMTTEADKPDADAMADVIIDDVSREKSMREDHKSFDDVIPAKKIIDWKGANPVTVINEYCQITRRDWSFRIESVGPSNSPTFYACVDIDGRVFDKADGKSKRDAKNNAAKLAVDKLLGYVIIRF

SEQ ID NO: 24 - DNA, 1026 bp, Ebola virus
atgacgacgcggacaaaaggccgaggccacactgcggtcacaacccagaatgacaggatgcccggtccggaattgtcaggctggatcagcgaacaacttatgacggggaggatacccgtaagtgatatattctgcgacatagagaataacccgggtctttgccacgtaagccagatgcaacagacaaaaccgaaccccaaaacgcgcaacagtcaaacccagaccgaccccatttgtaaccatagcttcgaagaggttgttcaaactctggcaagcctggctacagtcgtccaacaacagacaatagcttccgaaagcttggagcagcgcataacgtctttggaaaacggattgaaaccagtttacgacatggcgaaaactataagctcacttaacagggtctgtgcggaaatggtcgccaagtacgaccttctcgttatgactactggccgagcgaccgccactgccgcagcgacagaagcatattgggcagagcatggacagccacccccagggccttctttgtacgaggaatccaccatacgaggaaagatagaaagtagagatgaaaccgtgcctcagtctgtgcgagaggcgtttaacaatcttgactctacaacatcattgacagaagaaaacttcggtaaaccagacataagcgcgaaggaccttcgaaatatcatgtacgaccaccttccggggttcggaaccgcgtttcaccaactggttcaagtgatctgtaagctcggtaaagattctaatagtcttgacatcattcacgccgagtttcaggccagtctggcggagggggattcaccacagtgtgcacttatacaaattaccaaacgggtgccgattttccaggatgccgcgccccctgttattcacattcggtccaggggcgatatcccgagagcgtgccaaaagtccctgcgccctgtaccgcccagtccaaagattgaccgaggctgggtttgtgtcttccagttgcaggacggaaagacactcgggttgaagatatgataa

SEQ ID NO: 25 - RNA, 1026 nt, Ebola virus
augacgacgcggacaaaaggccgaggccacacugcggucacaacccagaaugacaggaugcccgguccggaauugucaggcuggaucagcgaacaacuuaugacggggaggauacccguaagugauauauucugcgacauagagaauaacccgggucuuugccacguaagccagaugcaacagacaaaaccgaaccccaaaacgcgcaacagucaaacccagaccgaccccauuuguaaccauagcuucgaagagguuguucaaacucuggcaagccuggcuacagucguccaacaacagacaauagcuuccgaaagcuuggagcagcgcauaacgucuuuggaaaacggauugaaaccaguuuacgacauggcgaaaacuauaagcucacuuaacagggucugugcggaaauggucgccaaguacgaccuucucguuaugacuacuggccgagcgaccgccacugccgcagcgacagaagcauauugggcagagcauggacagccacccccagggccuucuuuguacgaggaauccaccauacgaggaaagauagaaaguagagaugaaaccgugccucagucugugcgagaggcguuuaacaaucuugacucuacaacaucauugacagaagaaaacuucgguaaaccagacauaagcgcgaaggaccuucgaaauaucauguacgaccaccuuccgggguucggaaccgcguuucaccaacugguucaagugaucuguaagcucgguaaagauucuaauagucuugacaucauucacgccgaguuucaggccagucuggcggagggggauucaccacagugugcacuuauacaaauuaccaaacgggugccgauuuuccaggaugccgcgcccccuguuauucacauucgguccaggggcgauaucccgagagcgugccaaaagucccugcgcccuguaccgcccaguccaaagauugaccgaggcuggguuugugucuuccaguugcaggacggaaagacacucggguugaagauaugauaa

SEQ ID NO: 26 - protein, 340 aa, Ebola virus
MTTRTKGRGHTAVTTQNDRMPGPELSGWISEQLMTGRIPVSDIFCDIENNPGLCHVSQMQQTKPNPKTRNSQTQTDPICNHSFEEVVQTLASLATVVQQQTIASESLEQRITSLENGLKPVYDMAKTISSLNRVCAEMVAKYDLLVMTTGRATATAAATEAYWAEHGQPPPGPSLYEESTIRGKIESRDETVPQSVREAFNNLDSTTSLTEENFGKPDISAKDLRNIMYDHLPGFGTAFHQLVQVICKLGKDSNSLDIIHAEFQASLAEGDSPQCALIQITKRVPIFQDAAPPVIHIRSRGDIPRACQKSLRPVPPSPKIDRGWVCVFQLQDGKTLGLKI

SEQ ID NO: 27 - RNA, 375 nt, PRRS virus
augccgaauaauaacgguaagcaacagaagaagaaaaaggguaauggacaacccguuaaccaacuuugccagaugcuggguaagauaauugcgcaacaaaaccaaucaagggggaaggggccagggaagaagaauagaaagaaaaaucccgaaaaaccccauuuuccuuuggcaacggaggacgauguacgacaucauuucacccccagcgagaggcaacuuugucugaguagcauucaaaccgccuuuaaucaaggcgcugggacuugcgcccuuagugacucuggucgaaucucauacaccguagaauuuagucucccgacccagcauacuguucgacugauccgagccaccgccucuccuucugcuugauaa

SEQ ID NO: 28 - RNA, 639 nt, Homo sapiens
augguugcccauaaucagguagcugcggacaaugccgucuccaccgcggcugaaccucgccggaggcccgaaccgaguucaaguaguuccaguagucccgccgcuccugcuaggccgagacccugucccgccguuccagccccugcaccaggagauacacacuuuaggacuuuccggagucacgcggacuauagacgcaucacacgcgcaucugcguugcuggacgcgugcgguuucuacuggggaccucucaguguacacggggcucacgaaaggcuucgagcggagcccguugguacguuucuuguucgagacuccaggcagcgaaauugcuuuuuugcucucagcgucaagauggccuccggaccgacuucaaucagaguucacuuccaagcagguagguuucauuuggacggaucucgcgagaguuucgacugccucuuugaacuucuggagcauuauguugcugcgcccaggcgaauguugggugcgccccuccgccaacggaggguccgaccgcugcaagaguuguguaggcaacgcauuguagcuaccguugguagagaaaaccuggcuaggauuccucuuaacccggugcuucgggauuaucucuccagcuucccauuccaaauaugauaa

SEQ ID NO: 29 - RNA, 1965 nt, Homo sapiens
auguucacucuccaagauucuuacguuaaacagaugguaaugagcuugcggguuucagaguugcaaguucuuuuggguuacgccggacgaaauaaacacgggaggaaacacgaguugcuuacaaaggcucugcaucugcuuaaagcggggugcucaccagccguucagaugaaaauaaaagagcuguaucgccgacgguuuccucaaaaaaucaugaccccggcggacuugucaauuccuaaugugcacaguucucccaugcccgcgacccugucucccaguacaaucccacaacugaccuaugauggccacccugcgucaaguccucucuugccggucucacuucuggguccgaagcaugaguuggagcuuccucaucucacauccgcguugcacccuguccauccugacaucaaauugcaaaagcuuccauucuacgaccuccuggacgagcuuaucaagccaacgagccucgcgucugacaacucucagcgauuuagggagacgugcuucgcguucgcacugaccccgcaacagguacaacagaucagcucgucuauggauauaucaggcacgaagugcgacuucacuguccaaguacagcuucgguuuugccucucagaaacauccugcccucaggaggaucacuucccuccgaaccuuugcgucaaggugaacacgaagccauguucucucccgggauauuugccuccuaccaagaauggcguugagccuaaacgccccucccgaccgaucaacauuaccucauugguccgcuugucaacuaccgucccgaauacuauuguggugaguuggacagcagagauaggucgaaauuacucaauggcgguuuaucugguaaagcagcugagcagcacugucuugcugcagagacuccgcgccaaggguauacgaaacccggaucacucccgagcauugaucaaggaaaaacucacagcggauccagacagcgagauagccaccaccuccuugcggguuagccucuuguguccguuggguaagaugcgacugaccauuccuugcagggcacucacguguucacaucuccaauguuuugacgccacguuguauauacagaugaaugaaaagaaaccgacuuggguuuguccagugugugauaagaaagcaccauaugaacaucuuaucauagauggccuguuuauggagauucugaaguauugcacugauugugaugaaauccaguucaaagaagaugguacgugggcucccaugagaaguaaaaaggaggugcaagaaguuagcgcgagcuacaacgggguggaugggugcuugaguuccacuuuggaacaccaaguggccucccaucaccaguccagcaauaagaacaagaaggucgaaguaaucgaccugacuauugauucauccuccgacgaggaggaagaggaaccuagcgccaagcgcacaugcccuucacuuucucccacgucaccuuugaacaauaagggaauuuugucuuugccccaccaagcuuccccgguguccaggacaccaagccuccccgccguugauaccuccuauauaaauacgucacugauucaggacuaucgccacccauuucacaugaccccuaugcccuaugaucugcaaggacuggacuucuucccguuuuuguccggagauaaucagcauuacaacacuagucuucuugcagccgcggcagcggcgguaagcgaugaccaggaucuucugcacucuaguagauuuuucccguacaccuccucacaaauguuucuggaucagcuuagugcuggcggaagcacuucucucccgacuaccaacggcaguucauccggcucaaacucuagccucgugucuaguaauagucuuagagagagucauucacauaccguuacgaauaggucuucaaccgauaccgcgucaaucuuuggcauuauuccugauauaauaucacucgauugauaa

SEQ ID NO: 30 - DNA, 375 bp, PRRS virus
atgccgaataataacggtaagcaacagaagaagaaaaagggtaatggacaacccgttaaccaactttgccagatgctgggtaagataattgcgcaacaaaaccaatcaagggggaaggggccagggaagaagaatagaaagaaaaatcccgaaaaaccccattttcctttggcaacggaggacgatgtacgacatcatttcacccccagcgagaggcaactttgtctgagtagcattcaaaccgcctttaatcaaggcgctgggacttgcgcccttagtgactctggtcgaatctcatacaccgtagaatttagtctcccgacccagcatactgttcgactgatccgagccaccgcctctccttctgcttgataa

SEQ ID NO: 31 - protein, 123 aa, PRRS virus
MPNNNGKQQKKKKGNGQPVNQLCQMLGKIIAQQNQSRGKGPGKKNRKKNPEKPHFPLATEDDVRHHFTPSERQLCLSSIQTAFNQGAGTCALSDSGRISYTVEFSLPTQHTVRLIRATASPSA

SEQ ID NO: 32 - DNA, 7561 bp, VEE virus
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattcggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 33 - RNA, 7561 nt, VEE virus
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauucgguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaag

SEQ ID NO: 34 - DNA, 1653 bp, Firefly
atggaagatgccaaaaacattaagaagggcccagcgccattctacccactcgaagacgggaccgccggcgagcagctgcacaaagccatgaagcgctacgccctggtgcccggcaccatcgcctttaccgacgcacatatcgaggtggacattacctacgccgagtacttcgagatgagcgttcggctggcagaagctatgaagcgctatgggctgaatacaaaccatcggatcgtggtgtgcagcgagaatagcttgcagttcttcatgcccgtgttgggtgccctgttcatcggtgtggctgtggccccagctaacgacatctacaacgagcgcgagctgctgaacagcatgggcatcagccagcccaccgtcgtattcgtgagcaagaaagggctgcaaaagatcctcaacgtgcaaaagaagctaccgatcatacaaaagatcatcatcatggatagcaagaccgactaccagggcttccaaagcatgtacaccttcgtgacttcccatttgccacccggcttcaacgagtacgacttcgtgcccgagagcttcgaccgggacaaaaccatcgccctgatcatgaacagtagtggcagtaccggattgcccaagggcgtagccctaccgcaccgcaccgcttgtgtccgattcagtcatgcccgcgaccccatcttcggcaaccagatcatccccgacaccgctatcctcagcgtggtgccatttcaccacggcttcggcatgttcaccacgctgggctacttgatctgcggctttcgggtcgtgctcatgtaccgcttcgaggaggagctattcttgcgcagcttgcaagactataagattcaatctgccctgctggtgcccacactatttagcttcttcgctaagagcactctcatcgacaagtacgacctaagcaacttgcacgagatcgccagcggcggggcgccgctcagcaaggaggtaggtgaggccgtggccaaacgcttccacctaccaggcatccgccagggctacggcctgacagaaacaaccagcgccattctgatcacccccgaaggggacgacaagcctggcgcagtaggcaaggtggtgcccttcttcgaggctaaggtggtggacttggacaccggtaagacactgggtgtgaaccagcgcggcgagctgtgcgtccgtggccccatgatcatgagcggctacgttaacaaccccgaggctacaaacgctctcatcgacaaggacggctggctgcacagcggcgacatcgcctactgggacgaggacgagcacttcttcatcgtggaccggctgaaaagcctgatcaaatacaagggctaccaggtagccccagccgaactggagagcatcctgctgcaacaccccaacatcttcgacgccggggtcgccggcctgcccgacgacgatgccggcgagctgcccgccgcagtcgtcgtgctggaacacggtaaaaccatgaccgagaaggagatcgtggactatgtggccagccaggttacaaccgccaagaagctgcgcggtggtgttgtgttcgtggacgaggtgcctaaaggactgaccggcaagttggacgcccgcaagatccgcgagattctcattaaggccaagaagggcggcaagatcgccgtgtaa

SEQ ID NO: 35 - RNA, 1653 nt, Firefly
auggaagaugccaaaaacauuaagaagggcccagcgccauucuacccacucgaagacgggaccgccggcgagcagcugcacaaagccaugaagcgcuacgcccuggugcccggcaccaucgccuuuaccgacgcacauaucgagguggacauuaccuacgccgaguacuucgagaugagcguucggcuggcagaagcuaugaagcgcuaugggcugaauacaaaccaucggaucguggugugcagcgagaauagcuugcaguucuucaugcccguguugggugcccuguucaucgguguggcuguggccccagcuaacgacaucuacaacgagcgcgagcugcugaacagcaugggcaucagccagcccaccgucguauucgugagcaagaaagggcugcaaaagauccucaacgugcaaaagaagcuaccgaucauacaaaagaucaucaucauggauagcaagaccgacuaccagggcuuccaaagcauguacaccuucgugacuucccauuugccacccggcuucaacgaguacgacuucgugcccgagagcuucgaccgggacaaaaccaucgcccugaucaugaacaguaguggcaguaccggauugcccaagggcguagcccuaccgcaccgcaccgcuuguguccgauucagucaugcccgcgaccccaucuucggcaaccagaucauccccgacaccgcuauccucagcguggugccauuucaccacggcuucggcauguucaccacgcugggcuacuugaucugcggcuuucgggucgugcucauguaccgcuucgaggaggagcuauucuugcgcagcuugcaagacuauaagauucaaucugcccugcuggugcccacacuauuuagcuucuucgcuaagagcacucucaucgacaaguacgaccuaagcaacuugcacgagaucgccagcggcggggcgccgcucagcaaggagguaggugaggccguggccaaacgcuuccaccuaccaggcauccgccagggcuacggccugacagaaacaaccagcgccauucugaucacccccgaaggggacgacaagccuggcgcaguaggcaagguggugcccuucuucgaggcuaaggugguggacuuggacaccgguaagacacugggugugaaccagcgcggcgagcugugcguccguggccccaugaucaugagcggcuacguuaacaaccccgaggcuacaaacgcucucaucgacaaggacggcuggcugcacagcggcgacaucgccuacugggacgaggacgagcacuucuucaucguggaccggcugaaaagccugaucaaauacaagggcuaccagguagccccagccgaacuggagagcauccugcugcaacaccccaacaucuucgacgccggggucgccggccugcccgacgacgaugccggcgagcugcccgccgcagucgucgugcuggaacacgguaaaaccaugaccgagaaggagaucguggacuauguggccagccagguuacaaccgccaagaagcugcgcggugguguuguguucguggacgaggugccuaaaggacugaccggcaaguuggacgcccgcaagauccgcgagauucucauuaaggccaagaagggcggcaagaucgccguguaa

SEQ ID NO: 36 - DNA, 1698 bp, Respiratory syncytial virus
atggaactgctgatcctgaaggccaacgccatcaccaccatcctgaccgccgtgaccttctgcttcgccagcggccagaacatcaccgaggaattctaccagagcacctgcagcgccgtgagcaagggctacctgagcgccctgcggaccggctggtacaccagcgtgatcaccatcgagctgtccaacatcaaagaaaacaagtgcaacggcaccgacgccaaggtgaaactgatcaagcaggaactggacaagtacaagaacgccgtgaccgagctgcagctgctgatgcagagcacccccgccaccaacaaccgggccagaagagagctgccccggttcatgaactacaccctgaacaacgccaagaaaaccaacgtgaccctgagcaagaagcggaagcggcggagcgccatcgccagcggggtggccgtgtccaaggtgctgcacctggaaggcgaggtgaacaagatcaagtccgccctgctgtccaccaacaaggccgtggtgtccctgagcaacggcgtgagcgtgctgaccagcaaggtgctggatctgaagaactacatcgacaagcagctgctgcccatcgtgaacaagcagagctgcagcatcagcaacatcgagaccgtgatcgagttccagcagaagaacaaccggctgctggaaatcacccgggagttcagcgtgaacgccggcgtgaccacccccgtgagcacctacatgctgaccaacagcgagctgctgtccctgatcaatgacatgcccatcaccaacgaccagaaaaagctgatgagcaacaacgtgcagatcgtgcggcagcagagctactccatcatgagcatcatcaaagaagaggtgctggcctacgtggtgcagctgcccctgtacggcgtgatcgacaccccctgctggaagctgcacaccagccccctgtgcaccaccaacaccaaagagggcagcaacatctgcctgacccggaccgaccggggctggtactgcgacaacgccggcagcgtgagcttcttcccccaagccgagacctgcaaggtgcagagcaaccgggtgttctgcgacaccatgaacagcctgaccctgccctccgaggtgaacctgtgcaacgtggacatcttcaaccccaagtacgactgcaagatcatgacctccaagaccgacgtgagcagctccgtgatcacctccctgggcgccatcgtgagctgctacggcaagaccaagtgcaccgccagcaacaagaaccggggcatcatcaagaccttcagcaacggctgcgactacgtgagcaacaagggcgtggacaccgtgagcgtgggcaacacactgtactacgtgaataagcaggaaggcaagagcctgtacgtgaagggcgagcccatcatcaacttctacgaccccctggtgttccccagcgacgagttcgacgccagcatcagccaggtcaacgagaagatcaaccagagcctggccttcatccggaagtccgacgagctgctgcacaatgtgaatgccggcaagagcaccaccaatatcatgatcaccacaatcatcatcgtgatcattgtgatcctgctgtctctgattgccgtgggcctgctgctgtactgcaaggcccgcagcacccctgtgaccctgtccaaggaccagctgtccggcatcaacaatatcgccttctccaactga

SEQ ID NO: 37 - RNA, 1698 nt, RSV
auggaacugcugauccugaaggccaacgccaucaccaccauccugaccgccgugaccuucugcuucgccagcggccagaacaucaccgaggaauucuaccagagcaccugcagcgccgugagcaagggcuaccugagcgcccugcggaccggcugguacaccagcgugaucaccaucgagcuguccaacaucaaagaaaacaagugcaacggcaccgacgccaaggugaaacugaucaagcaggaacuggacaaguacaagaacgccgugaccgagcugcagcugcugaugcagagcacccccgccaccaacaaccgggccagaagagagcugccccgguucaugaacuacacccugaacaacgccaagaaaaccaacgugacccugagcaagaagcggaagcggcggagcgccaucgccagcgggguggccguguccaaggugcugcaccuggaaggcgaggugaacaagaucaaguccgcccugcuguccaccaacaaggccguggugucccugagcaacggcgugagcgugcugaccagcaaggugcuggaucugaagaacuacaucgacaagcagcugcugcccaucgugaacaagcagagcugcagcaucagcaacaucgagaccgugaucgaguuccagcagaagaacaaccggcugcuggaaaucacccgggaguucagcgugaacgccggcgugaccacccccgugagcaccuacaugcugaccaacagcgagcugcugucccugaucaaugacaugcccaucaccaacgaccagaaaaagcugaugagcaacaacgugcagaucgugcggcagcagagcuacuccaucaugagcaucaucaaagaagaggugcuggccuacguggugcagcugccccuguacggcgugaucgacacccccugcuggaagcugcacaccagcccccugugcaccaccaacaccaaagagggcagcaacaucugccugacccggaccgaccggggcugguacugcgacaacgccggcagcgugagcuucuucccccaagccgagaccugcaaggugcagagcaaccggguguucugcgacaccaugaacagccugacccugcccuccgaggugaaccugugcaacguggacaucuucaaccccaaguacgacugcaagaucaugaccuccaagaccgacgugagcagcuccgugaucaccucccugggcgccaucgugagcugcuacggcaagaccaagugcaccgccagcaacaagaaccggggcaucaucaagaccuucagcaacggcugcgacuacgugagcaacaagggcguggacaccgugagcgugggcaacacacuguacuacgugaauaagcaggaaggcaagagccuguacgugaagggcgagcccaucaucaacuucuacgacccccugguguuccccagcgacgaguucgacgccagcaucagccaggucaacgagaagaucaaccagagccuggccuucauccggaaguccgacgagcugcugcacaaugugaaugccggcaagagcaccaccaauaucaugaucaccacaaucaucaucgugaucauugugauccugcugucucugauugccgugggccugcugcuguacugcaaggcccgcagcaccccugugacccuguccaaggaccagcuguccggcaucaacaauaucgccuucuccaacuga

SEQ ID NO: 38 - DNA, 747 bp, Human enterovirus
ttaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgacc

SEQ ID NO: 39 - RNA, 747 nt, Human enterovirus
uuaaaacagcuguggguuguucccacccacagggcccacugggcgcuagcacucugauuuuacgaaauccuugugcgccuguuuuauaucccuucccuaauucgaaacguagaagcaaugcgcaccacugaucaauaguaggcguaacgcgccaguuacgucaugaucaagcauaucuguucccccggacugaguaucaauagacugcuuacgcgguugaaggagaaaacguucguuauccggcuaacuacuucgagaagcccaguaacaccauggaagcugcaggguguuucgcucagcacuucccccguguagaucaggucgaugagccacugcaauccccacaggugacuguggcaguggcugcguuggcggccugccuauggggagacccauaggacgcucuaauguggacauggugcgaagagucuauugagcuaguuaguaguccuccggccccugaaugcggcuaauccuaacugcggagcacaugccuucaacccagaggguagugugucguaacgggcaacucugcagcggaaccgacuacuuuggguguccguguuucuuuuuuauucuuauauuggcugcuuauggugacaauuacagaauuguuaccauauagcuauuggauuggccauccgguguguaauagagcuguuauauaccuauuuguuggcuuuguaccacuaacuuuaaaaucuauaacuacccucaacuuuauauuaacccucaauacaguugacc

(19) United States
(12) Patent Application Publication
(10) Pub. No.: US 2024/0301006 A1
(43) Pub. Date: Sep. 12, 2024

(54) SELF-AMPLIFYING MESSENGER RNA

(71) Applicant: GLAXOSMITHKLINE BIOLOGICALS SA, Rixeensart (BE)

(72) Inventors: Giulietta MARUGGI, Rockville, MD (US); Jason William WESTERBECK, Rockville, MD (US); Dong YU, Rockville, MD (US)

(73) Assignee: GLAXOSMITHKLINE BIOLOGICALS SA, Rixensart (BE)

(21) Appl. No.: 18/269,120

(22) PCT Filed: Dec. 21, 2021
(86) PCT No.: PCT/IB2021/062120

§ 371 Date: Jun. 22, 2023

Related U.S. Application Data
(60) Provisional application No. 63/129,837, filed on Dec. 23, 2020.

(51) Int. Cl. C07K 14/005; A61K 9/127; A61K 39/00; C12N 15/86

(52) U.S. Cl. CPC C07K 14/005; A61K 9/127; C12N 15/86; A61K 39/00; A61K 2039/55555; A61K 2039/572; A61K 2039/575; C12N 2710/24122; C12N 2710/24133; C12N 2760/14122; C12N 2760/14133; C12N 2760/16122; C12N 2760/16133; C12N 2770/36143

(57) ABSTRACT

Compounds useful as components of immunogenic compositions for the induction of an immunogenic response in a subject against infection, methods for their use in treatment, and processes for their manufacture are provided herein. The compounds comprise a nucleic acid construct comprising a sequence which encodes an interferon effector.

This application claims priority to U.S. provisional patent application U.S. Ser. No. 63/129,837, filed 23 Dec. 2020, which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on 21 Dec. 2021, is named VU66992WOPatentln_ST25.txt and is 106,000 bytes in size.

### FIELD OF THE INVENTION

Compositions and methods for modulating the interferon response to a ribonucleic acid (RNA) are provided.

### BACKGROUND TO THE INVENTION

RNAs are known to induce an interferon response when introduced into a cell. Messenger RNAs (mRNAs) introduced into a cell are known to induce an interferon response. Should the introduced mRNA undergo replication, this too can contribute to the induction of an interferon response (IFN). This may be pronounced in the case of a self-replicating mRNA or a trans-replicated mRNA. The interferon response may interfere with the function in vivo of a gene of interest introduced via mRNA. Alternatively, an elevated interferon response may be desirable in certain circumstances.

In the context of delivering a mRNA or a self-replicating RNA coding for a polypeptide antigen, an antigen-binding polypeptide, an immune-modulatory polypeptide, or a therapeutic polypeptide to a subject in vivo, there is a need to modulate the interferon responses.

### SUMMARY OF THE INVENTION

The present inventors provide mRNAs and self-amplifying mRNAs comprising sequences for modulating an interferon response, as well as the nucleic acids encoding them. Methods for their use in treatment, and processes for their manufacture are also provided.

In some embodiments, compositions are provided comprising a self-replicating (mRNA) comprising a construct encoding a heterologous polypeptide interferon effector that suppresses an interferon response, wherein the heterologous polypeptide interferon effector is VP35, or a variant or fragment thereof.

In some embodiments, compositions are provided comprising a self-replicating (mRNA) comprising a construct encoding a heterologous polypeptide interferon effector that suppresses an interferon response, wherein the heterologous polypeptide interferon effector is N, or a variant or fragment thereof.

In some embodiments, compositions are provided comprising self-replicating messenger RNA (mRNA) comprising a construct encoding two or more heterologous polypeptide interferon effectors that suppress an interferon response, wherein the heterologous polypeptide interferon effectors one or more of VP35, or a variant or fragment thereof; NS1, or a variant or fragment thereof; and E3, or a variant or fragment thereof.

In some embodiments, compositions are provided comprising a self-replicating mRNA comprising a construct encoding a heterologous polypeptide interferon effector that enhances an interferon response, wherein the heterologous polypeptide interferon effector is PB1-F2, or a variant or fragment thereof.

In some embodiments, compositions comprising a RNA molecule comprised from 5′ to 3′ of (a) a polynucleotide of SEQ ID NO:2 or SEQ ID NO:8, variants and fragments thereof; (b) a polynucleotide sequence encoding a polypeptide comprising the sequence of SEQ ID NO:17, SEQ ID NO:20; SEQ ID NO:23; or SEQ ID NO:26, variants and fragments thereof; and (c) a polynucleotide sequence comprising the sequence of SEQ ID NO:6, variants and fragments thereof are provided.

In some embodiments, the compositions further comprise a non-viral delivery system.

In some embodiments, DNA encoding the RNA molecules are provided. In some embodiments, processes for making the compositions and methods for their use are provided.

### DESCRIPTION OF DRAWINGS/FIGURES

FIG. 1A-FIG. 1C. FIG. 1A shows the percent of cells GFP (+) by FACS. FIG. 1B shows the levels of mean fluorescent intensity of GFP expression by FACS. FIG. 1C shows the expression of IFN-beta (by ELISA).

FIG. 2A-FIG. 2E. FIG. 2A shows the results of the C2C12 potency analysis, revealing that when compared to control SAM replicons expressing luciferase alone, or RSVF alone, as well as to a control SAM expressing the innate immune-inert influenza HA protein after the respective antigens, the SAM IFN modulating replicons expressing the corresponding antigens were similarly potent across multiple RNA concentrations. From left to right, eight constructs were tested:
1) SAM_SG-LUC_IRES-HA (luciferase under the SAM subgenomic promoter; HA under an IRES). RNA concentration was, left to right, 2000 ng/well; 667 ng/well; 222 ng/well; 74 ng/well; 8.23 ng/well; 2.7 ng/well; and 0.91 ng/well.
2) SAM(nsP3mut)_SG-Luc (SAM having mutations in nsP3 (SEQ ID NO:8) and luciferase under the subgenomic promoter). RNA concentration as above.
3) SAM_SG-Luc_IRES-VP35 (SAM encoding luciferase under the subgenomic promoter and VP35 under the IRES). RNA concentration as above.
4) SAM_SG-Luc_IRES-NS1 (SAM encoding luciferase under the subgenomic promoter and NS1 under the IRES). RNA concentration as above.
5) SAM_SG-Luc_IRES-E3 (SAM encoding luciferase under the subgenomic reporter and E3 under the IRES). RNA concentration as above.
6) SAM_SG-Luc_IRES-N(SAM encoding luciferase under the subgenomic reporter and N under the IRES). RNA concentration as above.
7) SAM_SG-Luc_IRES-PB1F2 (SAM encoding luciferase under the subgenomic reporter and PB1F2 under the IRES). RNA concentration as above.
8) SAM SG_Luc (SAM encoding luciferase under the subgenomic reporter). RNA concentration as above.

FIG. 2B compares SAM IFN modulating construct down regulation of IFN-β expression when viral proteins were expressed to the antigen alone. Each construct was tested in triplicate (left to right, 222 ng/well; 74 ng/well; 24 ng/well). (See 2A for construct content.)

FIG. 2C depicts the results of LNP formulated luciferase versions of the SAM IFN modulating replicons tested in C2C12 cells. (See 2A for construct content.)

FIG. 2D shows potency analysis in C2C12 cells of control SAM replicons expressing respiratory syncytial virus F antigen (RSVF) alone, a control SAM expressing the innate immune-inert influenza HA protein after the respective antigens. From left to right, the replicons expressed RSVF antigen: alone (A375); in SAM having mutations in nsP3 (pJW34); with the innate immune-inert influenza HA protein (pJW35); with VP35 (pJW37); with NS1 (pJW38); with E3 (pJW39); with N (pJW40); with PB1F2 (pJW41); an irrelevant antibody; cells. Each replicon was tested using 2000 ng/well; 667 ng/well; 222 ng/well; 74 ng/well; 8.23 ng/well; 2.7 ng/well; and 0.91 ng/well.

FIG. 2E shows analysis of the IFN-β expression levels by ELISA comparing SAM IFN modulating replicons RSVF constructs to the antigen. (See FIG. 2D for replicon content.) Each replicon was tested in triplicate (left to right, 222 ng/well; 74 ng/well; 24 ng/well).

FIG. 3A-FIG. 3H. FIG. 3A shows the results of the potency assay for SAM replicons expressing the luciferase antigen in HSKM cells, as measured by percent cells expressing the constructs.

FIG. 3B shows the results of the potency assay for SAM replicons expressing the luciferase antigen in HSKM cells, as measured by the mean total intensity of the cells in each well of the 96-well plate. See FIG. 2A for replicon content.

FIG. 3C shows the results of the IFN ELIZA assay for SAM replicons expressing the luciferase antigen in HSKM cells. See FIG. 2A for replicon content.

FIG. 3D shows down regulation of innate signalling factor by the IFN modulating SAM replicons for the inflammatory factor IL-6. FIG. 3E shows the same for IP-10; FIG. 3F, MCP-1; FIG. 3G, MIP1-β; and FIG. 3H, TNFα. See FIG. 2A for replicon content.

FIG. 4 shows the results of a study of luminescence overtime of IFN modulating replicons in mice. See FIG. 2A for replicon content.

FIG. 5A shows the results of a CD8 T cell immunity analysis from the spleens of mice vaccinated with SAM IFN modulating replicons expressing RSVF. In each case, the replicon expressed RSVF under the subgenomic promoter and, if IRES appears, the indicated antigen under control of an IRES.

FIG. 5A shows the results of a CD4 T cell immunity analysis from the spleens of mice vaccinated with SAM IFN modulating replicons expressing RSVF.

FIG. 6 shows neutralizing antibody (Nab) titers for IFN modulating replicons compared to the RSVF alone and RSVF-HA control constructs.

### DETAILED DESCRIPTION OF THE INVENTION

### Constructs: Variants: Fragments

The present inventors provide constructs comprising one or more coding regions for a heterologous polypeptide interferon effector, which constructs find use in mRNA and self-replicating mRNA comprising them. Such constructs may further comprise a coding region for a polypeptide antigen; an antigen-binding polypeptide; an immune-modulatory polypeptide; or a therapeutic polypeptide. A construct can be delivered to a subject as a RNA component of a mRNA or a self-replicating mRNA, or also refers to the nucleic acid, such as DNA, from which the RNA construct is transcribed. Thus, by “construct” is intended a nucleic acid that encodes polypeptide sequences described herein, and may comprise DNA, RNA, or non-naturally occurring nucleic acid monomers. The nucleic acid components of constructs are described more fully in the Nucleic Acids section herein.

In some embodiments, the constructs herein encode wild-type polypeptide sequences, or a variant, or a fragment thereof. In some embodiments, a construct may encode polypeptide sequences heterologous to each other.

A “variant” of a polypeptide sequence includes amino acid sequences having one or more amino acid substitutions and/or deletions when compared to the reference sequence. In some embodiments, a variant includes the relevant polypeptide from a TC-83 alpha viral vector. In some embodiments, the variant may comprise an amino acid sequence which is at least 70%, at least 75%, at least 80%, at least 85%, at least 90%, at least 95%, at least 96%, at least 97%, at least 98%, or at least 99%) identical to a full-length wild-type polypeptide. Alternatively, or in addition, a fragment of a polypeptide may comprise an interferon effector fragment of the full-length polypeptide which may comprise a contiguous amino acid sequence of at least 8, at least 9, at least 10, at least 11, at least 12, at least 13, at least 14, at least 15, at least 16, at least 17, at least 18, at least, or more amino acids which is identical to a contiguous amino acid sequence of the full-length polypeptide. As used herein, the term “antigen” refers to a molecule containing one or more epitopes (e.g., linear, conformational or both) that will stimulate a host's immune system to make a humoral and/or cellular antigen-specific immunological response (i.e. an immune response which specifically recognizes a naturally occurring polypeptide). An “epitope” is that portion of an antigen that determines its immunological specificity.

In some embodiments, constructs and self-replicating RNA molecules are provided herein that encode a heterologous polypeptide interferon effector. A “heterologous polypeptide interferon effector” includes wild-type viral or host cell proteins that alter or interrupt IFN functions, such as cytoplasmic RNA sensing, or hinder IFN signaling pathways, such as JAK-STAT, or a variant, or a fragment thereof.

By “VP35” is intended a polypeptide of the Ebola virus or a variant, or a fragment thereof. See Basler et al. (2003 J. Virol. 77:7945-7956.

By “N” is intended Porcine Reproductive and Respiratory Syndrome Virus N or a variant, or a fragment thereof. See Patel (2010) J. Virol. 84: 11045-11055.

By “NS1” is intended the NS1 polypeptide of influenza A or a variant, or a fragment thereof. See Koliopoulos et al. (2018). Nat Commun 9, 1820 By “PB1-F2” is intended a polypeptide of the 1918 pandemic influenza strain or a variant, or a fragment thereof. See Park et al. (2019) EMBO J 38.

It has been observed in vitro that application of purified viral interferon effector proteins to cells transfected with an mRNA or a self-replicating mRNA encoding a desired polypeptide can reduce unwanted IFN-mediated antiviral responses, thereby increasing the expression of the desired protein. See, for instance, Yoshioka et al. (2017) “Enhanced generation of iPSCs from older adult human cells by a synthetic five-factor self-replicative RNA.” PLoSOne 12, e0182018; Kim et al. (2017) “Recombinant Vaccinia virus-coded interferon inhibitor B18R: Expression, refolding and a use in a mammalian expression system with a RNA-vector” PLOSOne, 12(12): e0189308. It has also been observed that in cells transfected with a conventional mRNA encoding an interferon effector, the expression of a desired protein from a second mRNA or self-replicating mRNA is enhanced. See, for instance, Yoshioka et al. (2013) “Efficient Generation of Human iPSCs by a Synthetic Self-Replicative RNA” Cell Stem Cell 13, 246-254. A high degree of variability has been observed in the effectiveness from one interferon effector to another. Beissert et al. (2017) “Improvement of In Vivo Expression of Genes Delivered by Self-amplifying RNA Using Vaccinia Virus Immune Evasion Proteins” HUMAN GENE THERAPY, 28(12): 1138-1146; Blakney et al. (2020) “Innate Inhibiting Proteins Enhance Expression and Immunogenicity of Self-Amplifying RNA,” Molecular Therapy, https://doi.org/10.1016/j.ymthe.2020.11.011. See also Shattock, WO2020254804, published 24 Dec. 2020, in which 10 interferon effectors were screened, resulting in two that were unique in their ability to enhance expression in primary human cells. The applicants concluded that the “activity was not predictable given other [effectors] evaluated were thought to work through similar mechanisms.”

In one aspect, the present invention aims at providing a suit of heterologous polypeptide interferon effectors that can be delivered via a self-replicating mRNA, wherein the heterologous polypeptide interferon effectors suppress or enhance an interferon response, depending on the desired effect, without altering significantly the expression of the polypeptides expressed from the self-replicating mRNA. For instance, suppressing the interferon-mediated response in a subject receiving a self-replicating mRNA may be desirable where the self-replicating mRNA is not being used to deliver an antigen, or where the self-replicating mRNA is being used to deliver an antigen but a strong interferon response is not deemed necessary. On the other hand, enhancing the interferon response may be desirable where the self-replicating mRNA is being utilized for its adjuvanting properties, for instance in conjunction with another nucleic acid vector or a recombinant protein.

In some embodiments, compositions are provided comprising a self-replicating messenger mRNA comprising a construct encoding two or more heterologous polypeptide interferon effectors that suppress an interferon response, wherein the heterologous polypeptide interferon effectors are selected from the group consisting of:
(a) VP35, or a variant or fragment thereof;
(b) NS1, or a variant or fragment thereof; and
(c) E3, or a variant or fragment thereof.

Thus, in some embodiments, a self-replicating mRNA comprising a construct encoding VP35, or a variant or fragment thereof and NS1, or a variant or fragment thereof are provided. In some embodiments, a self-replicating mRNA comprising a construct encoding VP35, or a variant or fragment thereof and E3, or a variant or fragment thereof are provided. In some embodiments, a self-replicating mRNA comprising a construct encoding NS1, or a variant or fragment thereof and E3, or a variant or fragment thereof are provided.

In some embodiments, compositions are provided comprising a self-replicating (mRNA) comprising a construct encoding a heterologous polypeptide interferon effector that suppresses an interferon response, wherein the heterologous polypeptide interferon effector is VP35, or a variant or fragment thereof.

Suitable VP35, NS1, and E3 polypeptides comprise the amino acid sequences set forth herein as SEQ ID NO:26; SEQ ID NO:17; and SEQ ID NO:23, respectively. Suitable RNA molecules encoding these polypeptides comprise the polynucleotide sequences set forth as SEQ ID NO:25; SEQ ID NO:16; and SEQ ID NO:22. Suitable DNA molecules encoding these RNAs comprise the polynucleotide sequences set forth as SEQ ID NO:24; SEQ ID NO:15; and SEQ ID NO:21.

In some embodiments where the construct encodes a VP35, NS1, and E3 polypeptide, the construct encodes a polypeptide having an amino acid sequence selected from SEQ ID NO:26; SEQ ID NO:17; and SEQ ID NO:23, or a variant which is at least 70%, at least 75%, at least 80%, at least 85%, at least 90%, at least 95%, at least 96%, at least 97%, at least 98%, or at least 99% identical thereto. In some embodiments, the construct encodes a polypeptide which comprises a fragment of a full-length sequence selected from the group consisting of SEQ ID NO:26; SEQ ID NO:17; and SEQ ID NO:23, wherein the fragment comprises a contiguous stretch of the amino acid sequence of the full-length sequence up to 1, 2, 3, 4, 5, 6, 7, 8, 9, or 10 amino acids shorter than full-length sequence.

In some embodiments, the construct comprises a RNA nucleic acid sequence selected from the group consisting of SEQ ID NO:25; SEQ ID NO:16; and SEQ ID NO:22. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence selected from the group consisting of SEQ ID NO:25; SEQ ID NO:16; and SEQ ID NO:22. In some embodiments, the construct comprises a nucleic acid sequence which comprises a fragment of a full-length sequence selected from the group consisting of SEQ ID NO:25; SEQ ID NO:16; and SEQ ID NO:22, wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, the construct comprises a DNA nucleic acid sequence selected from the group consisting of SEQ ID NO:24; SEQ ID NO:15; and SEQ ID NO:21. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence selected from the group consisting of SEQ ID NO:24; SEQ ID NO:15; and SEQ ID NO:21. In some embodiments, the construct comprises a nucleic acid sequence which comprises a fragment of a full-length sequence selected from the group consisting of SEQ ID NO:24; SEQ ID NO:15; and SEQ ID NO:21, wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, compositions comprising a self-replicating mRNA comprising a construct encoding a heterologous polypeptide interferon effector that enhances an interferon response, wherein the heterologous polypeptide interferon effector is PB1-F2, or a variant or fragment thereof.

Suitable PB1-F2 polypeptides comprise the amino acid sequences set forth herein as SEQ ID NO:20. Suitable RNA molecules encoding this polypeptide comprise the polynucleotide sequences set forth as SEQ ID NO:19. Suitable DNA molecules encoding these RNAs comprise the polynucleotide sequence set forth as SEQ ID NO:18.

In some embodiments where the construct encodes a PB1-F2 polypeptide, the construct encodes a polypeptide having an amino acid sequence selected from SEQ ID NO:20, or a variant which is at least 70%, at least 75%, at least 80%, at least 85%, at least 90%, at least 95%, at least 96%, at least 97%, at least 98%, or at least 99% identical thereto. In some embodiments, the construct encodes a polypeptide which comprises a fragment of a full-length sequence selected from the group consisting of SEQ ID NO:20, wherein the fragment comprises a contiguous stretch of the amino acid sequence of the full-length sequence up to 1, 2, 3, 4, 5, 6, 7, 8, 9, or 10 amino acids shorter than full-length sequence.

In some embodiments, the construct comprises a RNA nucleic acid sequence of SEQ ID NO:19. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence selected from the group consisting of SEQ ID NO:19. In some embodiments, the construct comprises a nucleic acid sequence which comprises a fragment of a full-length sequence selected from the group consisting of SEQ ID NO:19, wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, the construct comprises a DNA nucleic acid sequence of SEQ ID NO:18. In some embodiments, the construct comprises a nucleic acid sequence which is at least 70%, at least 75%, at least 80%, at least 85%, at least 90%, at least 95%, at least 96%, at least 97%, at least 98%, or at least 99% identical to a sequence selected from the group consisting of SEQ ID NO:18. In some embodiments, the construct comprises a nucleic acid sequence which comprises a fragment of a full-length sequence selected from the group consisting of SEQ ID NO:18, wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

The constructs described above may further comprise one or more polynucleotide sequences encoding one or more polypeptides selected from the group consisting of: a polypeptide antigen; an antigen-binding polypeptide; an immune-modulatory polypeptide; or a therapeutic polypeptide. Where a construct comprises more than one polynucleotide sequence encoding more than one polypeptide, the polypeptides may be expressed as a single fusion protein or the polynucleotide sequences may be separated by control elements such as a subgenomic promoter. A suitable polynucleotide comprising a subgenomic promotor is set forth in SEQ ID NO:3 (DNA) and SEQ ID NO:4 (RNA).

### Nucleic Acids

Nucleic acid as disclosed herein can take various forms (e.g. single-stranded, double-stranded, vectors etc.). Nucleic acids may be circular or branched, but will generally be linear.

The nucleic acids used herein are preferably provided in purified or substantially purified form i.e. substantially free from other nucleic acids (e.g. free from naturally-occurring nucleic acids), particularly from reagents and enzymes, or production cell nucleic acids, generally being at least about 50% pure (by weight), and usually at least about 90% pure.

Nucleic acids may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap.

Typically, the nucleic acids of the invention will be in recombinant form, i. e. a form which does not occur in nature. For example, the nucleic acid may comprise one or more heterologous nucleic acid sequences (e.g. a sequence encoding another antigen and/or a control sequence such as a promoter or an internal ribosome entry site) in addition to the sequence encoding an expressed polypeptide. The nucleic acid may be part of a vector i.e. part of a nucleic acid designed for transduction/transfection of one or more cell types. Vectors may be, for example, “expression vectors” which are designed for expression of a nucleotide sequence in a host cell, or “viral vectors” which are designed to result in the production of a recombinant virus or virus-like particle.

Alternatively, or in addition, the sequence or chemical structure of the nucleic acid may be modified compared to a naturally-occurring sequence which encodes an expressed polypeptide. The sequence of the nucleic acid molecule may be modified, e.g. to increase the efficacy of expression or replication of the nucleic acid, or to provide additional stability or resistance to degradation.

The nucleic acid encoding the polypeptides described above may be codon optimized. In some embodiments, the nucleic acid encoding the polypeptides described above may be codon optimized for expression in human cells. By “codon optimized” is intended modification with respect to codon usage may increase translation efficacy and half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5) ppp (5) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increases translation efficacy.

The nucleic acids may comprise one or more nucleotide analogs or modified nucleotides. As used herein, “nucleotide analog” or “modified nucleotide” refers to a nucleotide that contains one or more chemical modifications (e.g., substitutions) in or on the nitrogenous base of the nucleoside (e.g., cytosine (C), thymine (T) or uracil (U)), adenine (A) or guanine (G)). A nucleotide analog can contain further chemical modifications in or on the sugar moiety of the nucleoside (e.g., ribose, deoxyribose, modified ribose, modified deoxyribose, six-membered sugar analog, or open-chain sugar analog), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases which can be incorporated into modified nucleosides and nucleotides and be present in the RNA molecules include: m5C (5-methylcytidine), m5U (5-methyluridine), m6A (N6-methyladenosine), s2U (2-thiouridine), Um (2′-O-methyluridine), mIA (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′im (I,2′-O-dimethylinosine); m3C (3-methylcytidine); Cm (2T-O-methylcytidine); s2C (2-thiocytidine); ac4C (N4-acetylcytidine); 5C (5-fonnylcytidine); m5Cm (5,2-O-dimethylcytidine); ac4Cm (N4acetyl2TOmethylcytidine); k2C (lysidine); mIG (1-methylguanosine); m2G (N2-methylguanosine); m7G (7-methylguanosine); Gm (2′-O-methylguanosine); m22G (N2,N2-dimethylguanosine); m2Gm (N2,2′-O-dimethylguanosine); m22Gm (N2,N2,2′-O-trimethylguanosine); Gr(p) (2′-O-ribosylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5s2U (5-aminomethyl-2-thiouridine); mnm5U (5-methylaminomethyluridine); mnm5s2U (5-methylaminomethyl-2-thiouridine); mnm5se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-O-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); 5Cm (5-formyl-2′-O-methylcytidine); mlGm (I 2′-O-dimethylguanosine); m′Am (1,2-O-dimethyl adenosine) irinomethyluridine); tm5s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, such as N1-methylpseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(C1-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides, such as with N1-methylpseudouridines (N1L), are available from commercial suppliers. In one embodiment, the RNA herein comprises at least one N1-methylpseudouridines (N14j).

Self-Replicating mRNA (SAM): SAM Vaccines

Self-replicating RNA molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells.

One suitable system for achieving self-replication in this manner is to use an alphavirus-based replicon. These replicons are +-stranded RNAs which lead to translation of a replicase (or replicase-transcriptase) after delivery to a cell. The replicase is translated as a polyprotein which auto-cleaves to provide a replication complex which creates genomic-strand copies of the +-strand delivered RNA. These −-strand transcripts can themselves be transcribed to give further copies of the +-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see the following reference: WO2005/113782.

In certain embodiments, the self-replicating RNA molecule described herein encodes (i) a RNA-dependent RNA polymerase which can transcribe RNA from the self-replicating RNA molecule and (ii) a construct as described above. The polymerase can be an alphavirus replicase e.g. comprising one or more of alphavirus proteins nsPI, nsP2, nsP3 and nsP4.

Whereas natural alphavirus genomes encode structural virion proteins in addition to the non-structural replicase polyprotein, in certain embodiments, the self-replicating RNA molecules do not encode alphavirus structural proteins. Thus, the self-replicating RNA can lead to the production of genomic RNA copies of itself in a cell, but not to the production of RNA-containing virions. The inability to produce these virions means that, unlike a wild-type alphavirus, the self-replicating RNA molecule cannot perpetuate itself in infectious form. The alphavirus structural proteins which are necessary for perpetuation in wild-type viruses are absent from self-replicating RNAs of the present disclosure and their place is taken by gene(s) encoding the immunogen of interest, such that the subgenomic transcript encodes the immunogen rather than the structural alphavirus virion proteins.

Thus a self-replicating RNA molecule useful with the invention may have two open reading frames. The first (5) open reading frame encodes a replicase; the second (3′) open reading frame encodes an antigen. In some embodiments the RNA may have additional (e.g. downstream) open reading frames e.g. to encode further antigens or to encode accessory polypeptides.

An empty TC83 self-replicating mRNA would comprise from 5′ to 3′ the polynucleotide sequence of SEQ ID NO:2 (and SEQ ID NO:4 if a subgenomic promoter were present), and SEQ ID NO:6. A DNA encoding an empty TC83 self-replicating mRNA would comprise from 5′ to 3′ the polynucleotide sequence of SEQ ID NO:1 (and SEQ ID NO:3 if a subgenomic promoterwere present), and SEQ ID NO:5. A construct would be inserted in between (and after the subgenomic promotor, if present) in order to express a heterologous polypeptide.

In certain embodiments, the self-replicating RNA molecule disclosed herein has a 5′ cap (e.g. a 7-methylguanosine). This cap can enhance in vivo translation of the RNA. In some embodiments the 5′ sequence of the self-replicating RNA molecule must be selected to ensure compatibility with the encoded replicase. In certain embodiments the first 5′ ribonucleotide after the 5′ cap comprises a 2′-methyl group on the ribose (cap1).

A self-replicating RNA molecule may have a 3′ poly-A tail. It may also include a poly-A polymerase recognition sequence (e.g. AAUAAA) near its 3′ end.

Self-replicating RNA molecules can have various lengths, but they are typically 5000-25000 nucleotides long. Self-replicating RNA molecules will typically be single-stranded. Single-stranded RNAs can generally initiate an adjuvant effect by binding to TLR7, TLR8, RNA helicases and/or PKR. RNA delivered in double-stranded form (dsRNA) can bind to TLR3, and this receptor can also be triggered by dsRNA which is formed either during replication of a single-stranded RNA or within the secondary structure of a single-stranded RNA.

The self-replicating RNA can conveniently be prepared by in vitro transcription (IVT). IVT can use a (cDNA) template created and propagated in plasmid form in bacteria, or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-replicating RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-replicating RNA can include (in addition to any 5′ cap structure) one or more nucleotides having a modified nucleobase. A RNA used with the invention ideally includes only phosphodiester linkages between nucleosides, but in some embodiments it can contain phosphoramidate, phosphorothioate, and/or methylphosphonate linkages.

The self-replicating RNA molecule may include a construct, as described elsewhere herein. Thus, the self-replicating RNA molecules described herein may be engineered to express multiple nucleotide sequences, from two or more open reading frames, thereby allowing co-expression of proteins, such as one, two or more together with cytokines or other immunomodulators, which can enhance the generation of an immune response. Such a self-replicating RNA molecule might be particularly useful, for example, in the production of various gene products (e.g., proteins) at the same time, for example, as a bivalent or multivalent vaccine.

If desired, the self-replicating RNA molecules can be screened or analyzed to confirm their therapeutic and prophylactic properties using various in vitro or in vivo testing methods that are known to those of skill in the art. For example, vaccines comprising self-replicating RNA molecule can be tested for their effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a self-replicating RNA molecule that encodes an T-cell epitope. In addition, T helper cell differentiation can be analyzed by measuring proliferation or production of TH1 (IL-2 and IFN-γ) and/or TH2 (IL-4 and IL-5) cytokines by ELISA or directly in CD4+ T cells by cytoplasmic cytokine staining and flow cytometry.

Self-replicating RNA molecules that encode a antigen can also be tested for ability to induce humoral immune responses, as evidenced, for example, by induction of B cell production of antibodies specific for a antigen of interest. These assays can be conducted using, for example, peripheral B lymphocytes from immunized individuals. Such assay methods are known to those of skill in the art. Other assays that can be used to characterize the self-replicating RNA molecules can involve detecting expression of the encoded antigen by the target cells. For example, FACS can be used to detect antigen expression on the cell surface or intracellularly. Another advantage of FACS selection is that one can sort for different levels of expression; sometimes-lower expression may be desired. Other suitable method for identifying cells which express a particular antigen involve panning using monoclonal antibodies on a plate or capture using magnetic beads coated with monoclonal antibodies.

In some embodiments, the self-replicating RNA molecules themselves comprises modified sequence that modulates the self-replicating mRNA response to interferon. In some embodiments, the self-replicating mRNA comprises a NSP3 region and the modified sequence comprises an amino acid substitution within the NSP3 region. In some embodiments, the NSP3 region encodes an E1595D amino acid substitution, a V1645M amino acid substitution, or both. See Li et al. (2019) “In vitro evolution of enhanced RNA replicons for immunotherapy,” Sci Rep 9, 6932. A suitable 5′ portion of a modified self-replicating mRNA having both is set forth in SEQ ID NO:8 (RNA) and SEQ ID NO:7 (DNA). A suitable 3′ portion would be unmodified and have the same TC83 sequence as described above.

In some embodiments, the self-replicating RNA molecules comprise from 5′ to 3′ a sequence which is at least 70%, at least 75%, at least 80%, at least 85%, at least 90%, at least 95%, at least 96%, at least 97%, at least 98%, or at least 99% identical to SEQ ID NO:2 or SEQ ID NO:8, a RNA construct, and a sequence which is at least 70%, at least 75%, at least 80%, at least 85%, at least 90%, at least 95%, at least 96%, at least 97%, at least 98%, or at least 99% identical to SEQ ID NO:6. In some embodiments, the self-replicating RNA molecule comprises from 5′ to 3′ a sequence that is a fragment of SEQ ID NO:2 or SEQ ID NO:8, a RNA construct, and a sequence that is a fragment of SEQ ID NO:6, wherein a fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, a DNA sequence encoding a self-replicating RNA molecule is provided, said DNA sequence comprising from 5′ to 3′ a DNA sequence having SEQ ID NO:1 or SEQ ID NO:7, a DNA construct, and a DNA sequence having SEQ ID NO:5. In some embodiments, a DNA sequence encoding a self-replicating RNA molecule is provided, said DNA sequence comprising from 5′ to 3′ a sequence which is at least 70%, at least 75%, at least 80%, at least 85%, at least 90%, at least 95%, at least 96%, at least 97%, at least 98%, or at least 99% identical to having SEQ ID NO:1 or SEQ ID NO:7, a DNA construct, and a DNA sequence which is at least 70%, at least 75%, at least 80%, at least 85%, at least 90%, at least 95%, at least 96%, at least 97%, at least 98%, or at least 99% identical to SEQ ID NO:5. In some embodiments, the DNA sequence encoding a self-replicating RNA molecule comprises from 5′ to 3′ a sequence that is a fragment of having SEQ ID NO:1 or SEQ ID NO:7, a DNA construct, and a sequence that is a fragment of SEQ ID NO:5, wherein the fragment comprises a contiguous stretch of the nucleic acid sequence of the full-length sequence up to 1, 2, 3, 4, 5, 6, 7, 8, 9, 10, 15, 20, 25, or 30 nucleic acids shorter than full-length sequence.

In some embodiments, compositions are provided comprising the self-replicating mRNA described herein, further comprising a non-viral delivery system. The delivery system (also referred to herein as a delivery vehicle) may have adjuvant effects which enhance the immunogenicity an encoded antigen. For example, the nucleic acid molecule may be encapsulated in liposomes, non-toxic biodegradable polymeric microparticles or viral replicon particles (VRPs), or complexed with particles of a cationic oil-in-water emulsion. In some embodiments, the nucleic acid-based vaccine comprises a cationic nano-emulsion (CNE) delivery system or a lipid nanoparticle (LNP) delivery system. In some embodiments, the nucleic acid-based vaccine comprises a non-viral delivery system, i.e., the nucleic acid-based vaccine is substantially free of viral capsid. Alternatively, the nucleic acid-based vaccine may comprise viral replicon particles. In other embodiments, the nucleic acid-based vaccine may comprise a naked nucleic acid, such as naked RNA (e.g. mRNA), but delivery via CNEs or LNPs, especially LNP, is preferred.

In certain embodiments, the nucleic acid-based vaccine comprises a cationic nano-emulsion (CNE) delivery system. CNE delivery systems and methods for their preparation are described in the following reference: WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in the following references: WO2012/006380; WO2013/006834; and WO2013/006837 (the contents of each of which are incorporated herein in their entirety).

Thus, in a nucleic acid-based vaccine of the invention, an RNA molecule herein may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non-viral delivery system, such as CNE, and the nucleic acid-based vaccine comprises a self-replicating RNA (mRNA). This may be particularly effective in eliciting humoral and cellular immune responses. Advantages also include the absence of a limiting anti-vector immune response and a lack of risk of genomic integration.

LNP delivery systems and non-toxic biodegradable polymeric microparticles, and methods for their preparation are described in the following references: WO2012/006376 (LNP and microparticle delivery systems); Geall et al. (2012) PNAS USA. September 4; 109(36): 14604-9 (LNP delivery system); and WO2012/006359 (microparticle delivery systems). LNPs are non-virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in the following references: WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

### Pharmaceutical Compositions; Immunogenic Compositions

The disclosure provides compositions comprising self-replicating mRNA and a non-viral delivery system. The composition may further be a pharmaceutical composition, e.g., the composition may also comprise a pharmaceutically acceptable carrier.

A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The compositions may also contain a pharmaceutically acceptable diluent, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier.

Pharmaceutical compositions may include the constructs, nucleic acid sequences, and/or polypeptide sequences described elsewhere herein in plain water (e.g. “w.f.i.”) or in a buffer e.g. a phosphate buffer, a Tris buffer, a borate buffer, a succinate buffer, a histidine buffer, or a citrate buffer. Buffer salts will typically be included in the 5-20 mM range. Pharmaceutical compositions may have a pH between 5.0 and 9.5 e.g. between 6.0 and 8.0. Compositions may include sodium salts (e.g. sodium chloride) to give tonicity. A concentration of 10±2 mg/mL NaCl is typical, e.g. about 9 mg/mL. Compositions may include metal ion chelators. These can prolong RNA stability by removing ions which can accelerate phosphodiester hydrolysis. Thus a composition may include one or more of EDTA, EGTA, BAPTA, pentetic acid, etc. Such chelators are typically present at between 10-500 uU e.g. 0.1 mM. A citrate salt, such as sodium citrate, can also act as a chelator, while advantageously also providing buffering activity. Pharmaceutical compositions may have an osmolality of between 200 mOsm/kg and 400 mOsm/kg, e.g. between 240-360 mOsm/kg, or between 290-310 mOsm/kg. Pharmaceutical compositions may include one or more preservatives, such as thiomersal or 2-phenoxyethanol. Mercury-free compositions are preferred, and preservative-free vaccines can be prepared. Pharmaceutical compositions may be aseptic or sterile. Pharmaceutical compositions may be non-pyrogenic e.g. containing <1 EU (endotoxin unit, a standard measure) per dose, and preferably <0.1 EU per dose. Pharmaceutical compositions may be gluten free. Pharmaceutical compositions may be prepared in unit dose form. In some embodiments a unit dose may have a volume of between 0.1-1.0 mL e.g. about 0.5 mL.

In some embodiments, the compositions disclosed herein are immunogenic composition that, when administered to a subject, induce a humoral and/or cellular antigen-specific immune response (i.e. an immune response which specifically recognizes a naturally occurring antigenic polypeptide). For example, an immunogenic composition may induce a memory T and/or B cell population relative to an untreated subject following an infection, particularly in those embodiments where the composition comprises a nucleic acid comprising a sequence which encodes an antigen. In some embodiments, the subject is a vertebrate, such as a mammal e.g. a human or a veterinary mammal.

The compositions of the invention can be formulated as vaccine compositions. The vaccine will comprise an immunologically effective amount of antigen. By “an immunologically effective amount” is intended that the administration of that amount to a subject, either in a single dose or as part of a series, is effective for inducing a measurable immune response against the antigen in the subject. This amount varies depending upon the health and physical condition of the individual to be treated, age, the taxonomic group of individual to be treated (e.g. human, non-human primate, etc.), the capacity of the individual's immune system to synthesize antibodies, the degree of protection desired, the formulation of the composition or vaccine, the treating doctor's assessment of the medical situation, the severity of the disease, the potency of the compound administered, the mode of administration, and other relevant factors. It is expected that the amount will fall in a relatively broad range that can be determined through routine trials. Vaccines as disclosed herein may either be prophylactic (i.e. to prevent infection) or therapeutic (i.e. to treat infection), but will typically be prophylactic. In some embodiments, the vaccine compositions disclosed herein may induce an effective immune response against a pathogen expressing the antigen, i.e., a response sufficient for treatment or prevention of a pathogenic infection.

### Methods of Use/Uses

In some embodiments, methods are provided for effecting or modulating an interferon response in a cell of a subject in need thereof. In some embodiments, methods are provided for inducing a protective or therapeutic immunological response to an antigen in a subject by administering a composition comprising a self-replicating mRNA encoding an antigen to the subject. In some embodiments there is provided the use of the construct or composition as disclosed herein in the manufacture of a medicament, such as a medicament for use in therapy or prevention. In some embodiments are provided use of the construct or composition as disclosed herein in the manufacture of a medicament for inducing an immune response to a pathogen in a subject. By “subject” is intended a vertebrate, such as a mammal e.g. a human or a veterinary mammal. In some embodiments the subject is human. Also provided is a construct or composition as disclosed herein for use as a medicament, such as for use in the inducing an immune response to a pathogen in a subject. A self-replicating mRNA encoding an antigen or compositions comprising a self-replicating mRNA encoding an antigen are provided for inducing a protective or therapeutic immunological response to an antigen in a subject.

### Routes of Administration/Dosages

Compositions disclosed herein will generally be administered directly to a subject. Direct delivery may be accomplished by parenteral injection, typically intramuscularly.

A dose of a nucleic acid (e.g. a nucleic acid-based vaccine) may have <10 (ug nucleic acid; e.g. from 0.001-10 ug, such as about 1 ug, 2.5 ug, 5 ug, 7.5 ug or 10 ug, but expression can be seen at much lower levels; e.g. using <1 ug/dose, <100 ng/dose, <10 ng/dose, <1 ng/dose, etc. Similarly, a dose of a protein antigen may have <10 ug protein; e.g. from 1-10 ug, such as about 1 ug, 2.5 ug, 5 ug, 7.5 ug or 10 ug.

### Processes of Manufacture/Formulation

Processes for the manufacture of self-replicating RNA are provided herein. In some embodiments, the process of manufacturing a self-replicating RNA comprises a step of in vitro transcription (IVT) as described elsewhere herein. In some embodiments, the process of manufacturing a self-replicating RNA comprises a step of IVT to produce a RNA, and further comprises a step of combining the RNA with a non-viral delivery system as described elsewhere herein. In some embodiments, the process of manufacturing a self-replicating RNA comprises a step of IVT to produce a RNA, and further comprises a step of combining the RNA with a CNE delivery system as described elsewhere herein.

### Sequence Identity

Identity or homology with respect to a sequence is defined herein as the percentage of amino acid residues in the candidate sequence that are identical with the reference amino acid sequence after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the shorter sequences in order to align the two sequences.

Where the present disclosure refers to a sequence by reference to a UniProt or Genbank accession code, the sequence referred to is the current version at the filing date of the present application.

### Embodiments

1. A composition comprising a self-replicating (mRNA) comprising a construct encoding a heterologous polypeptide interferon effector that suppresses an interferon response, wherein the heterologous polypeptide interferon effector is VP35, or a variant or fragment thereof.

1A. A composition comprising a self-replicating messenger RNA (mRNA) comprising a construct encoding two or more heterologous polypeptide interferon effectors that suppress an interferon response, wherein the heterologous polypeptide interferon effectors comprise VP35, or a variant or fragment thereof, and N, or a variant or fragment thereof.

1B. A composition comprising a self-replicating messenger RNA (mRNA) comprising a construct encoding two or more heterologous polypeptide interferon effectors that suppress an interferon response, wherein the heterologous polypeptide interferon effectors comprise VP35, or a variant or fragment thereof, and NS1, or a variant or fragment thereof.

1C. A composition comprising a self-replicating messenger RNA (mRNA) comprising a construct encoding two or more heterologous polypeptide interferon effectors that suppress an interferon response, wherein the heterologous polypeptide interferon effectors comprise VP35, or a variant or fragment thereof, and E3, or a variant or fragment thereof.

1D. A composition comprising a self-replicating (mRNA) comprising a construct encoding a heterologous polypeptide interferon effector that suppresses an interferon response, wherein the heterologous polypeptide interferon effector is N, or a variant or fragment thereof.

1E. A composition comprising a self-replicating messenger RNA (mRNA) comprising a construct encoding two or more heterologous polypeptide interferon effectors that suppress an interferon response, wherein the heterologous polypeptide interferon effectors comprise N, or a variant or fragment thereof, and NS1, or a variant or fragment thereof.

1F. A composition comprising a self-replicating messenger RNA (mRNA) comprising a construct encoding two or more heterologous polypeptide interferon effectors that suppress an interferon response, wherein the heterologous polypeptide interferon effectors comprise N, or a variant or fragment thereof, and E3, or a variant or fragment thereof.

2. A composition comprising a self-replicating messenger RNA (mRNA) comprising a construct encoding two or more heterologous polypeptide interferon effectors that suppress an interferon response, wherein the heterologous polypeptide interferon effectors are selected from the group consisting of:
(a) VP35, or a variant or fragment thereof;
(b) N, or a variant or fragment thereof;
(c) NS1, or a variant or fragment thereof; and
(d) E3, or a variant or fragment thereof.

3. A composition comprising a self-replicating mRNA comprising a construct encoding a heterologous polypeptide interferon effector that enhances an interferon response, wherein the heterologous polypeptide interferon effector is PB1-F2, or a variant or fragment thereof.

4. The composition of embodiments 1-3, wherein the self-replicating mRNA comprises modified sequence that modulates the self-replicating mRNA response to interferon.

5. The composition of embodiment 4, wherein the self-replicating mRNA comprises a NSP3 region and the modified sequence comprises an amino acid substitution within the NSP3 region.

6. The composition of embodiment 5, wherein the NSP3 region is a TC83 NSP3 region that encodes an E1595D amino acid substitution, a V1645M amino acid substitution, or both.

7. The composition of embodiment 6, wherein the RNA sequence encoding the amino acid substitution within the NSP3 region comprises a G4796U nucleotide substitution, a G4944A nucleotide substitution, or both.

8. The composition of any of embodiments 1-7, wherein the self-replicating mRNA further comprises a construct encoding a polypeptide selected from the group consisting of: a polypeptide antigen; an antigen-binding polypeptide; an immune-modulatory polypeptide; or a therapeutic polypeptide.

9. The composition of embodiment 8, wherein the construct encodes two or more polypeptides selected from the group.

10. The composition of embodiments 8-9, wherein the coding region does not encode luciferase or green fluorescent protein (GFP).

11. A composition comprising a RNA molecule, the RNA molecule comprising from 5′ to 3′:
(a) a polynucleotide sequence comprising (i) the sequence of SEQ ID NO:2, (ii) a polynucleotide having at least 90% identity to SEQ ID NO:2, or (iii) a fragment of SEQ ID NO:2 that is up to 30 nucleic acids shorter than full-length sequence;
(b) a construct comprising one or more polynucleotide sequences encoding a polypeptide selected from the group consisting of (i) a polypeptide comprising the sequence of SEQ ID NO:17, SEQ ID NO:20; SEQ ID NO:23; or SEQ ID NO:26, (ii) a polypeptide having at least 90% identity to SEQ ID NO:17, SEQ ID NO:20, SEQ ID NO:23, or SEQ ID NO:26, or (iii) a polypeptide comprising a fragment of SEQ ID NO:17, SEQ ID NO:20, SEQ ID NO:23, or SEQ ID NO:26 that is up to 10 amino acids shorter than full-length sequence; and
(c) a polynucleotide sequence comprising (i) the sequence of SEQ ID NO:6, (ii) a polynucleotide having at least 90% identity to SEQ ID NO:6, or (iii) a fragment of SEQ ID NO:6 that is up to 30 nucleic acids shorter than full-length sequence.

12. A composition comprising a RNA molecule, the RNA molecule comprising from 5′ to 3′:
(a) a polynucleotide sequence comprising (i) the sequence of SEQ ID NO:8, (ii) a polynucleotide having at least 90% identity to SEQ ID NO:8, or (iii) a fragment of SEQ ID NO:8 that is up to 30 nucleic acids shorter than full-length sequence;
(b) a construct comprising one or more polynucleotide sequences encoding a polypeptide selected from the group consisting of (i) a polypeptide comprising the sequence of SEQ ID NO:17, SEQ ID NO:20; SEQ ID NO:23; or SEQ ID NO:26, (ii) a polypeptide having at least 90% identity to SEQ ID NO:17, SEQ ID NO:20, SEQ ID NO:23, or SEQ ID NO:26, or (iii) a polypeptide comprising a fragment of SEQ ID NO:17, SEQ ID NO:20, SEQ ID NO:23, or SEQ ID NO:26 that is up to 10 amino acids shorter than full-length sequence; and
(c) a polynucleotide sequence comprising (i) the sequence of SEQ ID NO:6, (ii) a polynucleotide having at least 90% identity to SEQ ID NO:6, or (iii) a fragment of SEQ ID NO:6 that is up to 30 nucleic acids shorter than full-length sequence.

13. The composition of embodiments 11-12, wherein the heterologous polypeptide interferon effector suppresses an innate interferon response and the construct of (b) comprising one or more polynucleotide sequences encoding a polypeptide selected from the group consisting of (i) a polypeptide comprising the sequence of SEQ ID NO:17, SEQ ID NO:23; or SEQ ID NO:26, (ii) a polypeptide having at least 90% identity to SEQ ID NO:17, SEQ ID NO:23, or SEQ ID NO:26, or (iii) a polypeptide comprising a fragment of SEQ ID NO:17, SEQ ID NO:23, or SEQ ID NO:26 that is up to 10 amino acids shorter than full-length sequence.

14. The composition of embodiments 11-12, wherein the heterologous polypeptide interferon effector enhances an innate interferon response and the construct of (b) comprising one or more polynucleotide sequences encoding a polypeptide selected from the group consisting of (i) a polypeptide comprising the sequence of SEQ ID NO:20; (ii) a polypeptide having at least 90% identity to SEQ ID NO:20, or (iii) a polypeptide comprising a fragment of SEQ ID NO:20 that is up to 10 amino acids shorter than full-length sequence.

15. The composition of embodiments 13-14, wherein the construct further encodes one or more polypeptides selected from the group consisting of: a polypeptide antigen; an antigen-binding polypeptide; an immune-modulatory polypeptide; or a therapeutic polypeptide between the polynucleotide sequence of (a) and the polynucleotide sequence (b) between the polynucleotide sequence of (b) and the polynucleotide sequence (c), or both.

16. A composition comprising a RNA molecule, the RNA molecule comprising from 5′ to 3′:
(a) a polynucleotide sequence comprising (i) the sequence of SEQ ID NO:8, (ii) a polynucleotide having at least 90% identity to SEQ ID NO:8, or (iii) a fragment of SEQ ID NO:8 that is up to 30 nucleic acids shorter than full-length sequence;
(b) a RNA construct encoding one or more polypeptides selected from the group consisting of: a polypeptide antigen; an antigen-binding polypeptide; an immune-modulatory polypeptide; or a therapeutic polypeptide; and
(c) a polynucleotide sequence comprising (i) the sequence of SEQ ID NO:6, (ii) a polynucleotide having at least 90% identity to SEQ ID NO:6, or (iii) a fragment of SEQ ID NO:6 that is up to 30 nucleic acids shorter than full-length sequence.

17. The composition of any of embodiments 1-16, wherein the composition comprises a non-viral delivery material.

18. The composition of embodiment 17, wherein the non-viral delivery material comprises a submicron cationic oil-in-water emulsion; a liposome; or a biodegradable polymeric microparticle delivery system.

19. The composition of embodiment 18, wherein the composition comprises a submicron cationic oil-in-water emulsion.

20. The composition according to embodiment 18, wherein the composition comprises a liposome.

21. The composition of embodiments 1-20 wherein the self-replicating mRNA comprises a construct encoding a polypeptide antigen and can induce an immunological response to the antigen in a subject when administered by intramuscular injection.

22. A DNA molecule encoding the self-replicating RNA of embodiments 1-21.

23. A process for producing an interferon effecting or modulating RNA comprising a step of transcribing the DNA molecule of embodiment 24 to produce a RNA molecule.

24. The process of embodiment 23, wherein the transcription is in vitro.

25. The process of embodiment 23, wherein the transcription is carried out by combining the DNA, a RNA polymerase, and nucleotides in a reaction.

26. The process of embodiment 23, wherein the transcription is carried out by combining the DNA with a cellular extract.

27. The process of embodiments 23, wherein said transcription is in vivo.

28. The process of embodiment 23-27, further comprising a step of formulating the RNA with a non-viral delivery system.

29. A composition produced by the process of any one of embodiments 23-28.

30. A method of effecting or modulating an interferon response in a cell of a subject in need thereof, which comprises administering to said subject the composition of embodiments 1-21 and 29.

31. A method of inducing a protective or therapeutic immunological response to an antigen in a subject by administering the composition of embodiment 21 to the subject.

32. The method of embodiments 30-31, wherein the subject is human.

33. The composition of embodiments 1-21 and 29, for use as a medicament, such as in therapy or prevention.

34. Use of the composition of embodiments 1-21 and 29 for effecting or modulating the IFN response in a subject.

35. A composition comprising a liposome comprising a mRNA encoding VP35, or a variant or fragment thereof.

36. The composition of embodiment 35, wherein the mRNA further comprises sequence recognized by a replicase encoded by a self-replicating mRNA, such that the mRNA can be amplified in trans by in the presence of the replicase.

37. The composition of embodiment 36, further comprising a self-replicating RNA molecule.

38. The composition of embodiments 1-21 and 29, further comprising a nucleic acid vector encoding a polypeptide antigen or a recombinant polypeptide antigen.

39. A composition comprising a mRNA comprising a construct encoding a heterologous polypeptide interferon effector that suppresses an interferon response, wherein the heterologous polypeptide interferon effector is VP35, or a variant or fragment thereof.

39A. A composition comprising a mRNA comprising a construct encoding two or more heterologous polypeptide interferon effectors that suppress an interferon response, wherein the heterologous polypeptide interferon effectors comprise VP35, or a variant or fragment thereof, and N, or a variant or fragment thereof.

39B. A composition comprising a mRNA comprising a construct encoding two or more heterologous polypeptide interferon effectors that suppress an interferon response, wherein the heterologous polypeptide interferon effectors comprise VP35, or a variant or fragment thereof, and NS1, or a variant or fragment thereof.

39C. A composition comprising a mRNA comprising a construct encoding two or more heterologous polypeptide interferon effectors that suppress an interferon response, wherein the heterologous polypeptide interferon effectors comprise VP35, or a variant or fragment thereof, and E3, or a variant or fragment thereof.

39D. A composition comprising a mRNA comprising a construct encoding a heterologous polypeptide interferon effector that suppresses an interferon response, wherein the heterologous polypeptide interferon effector is N, or a variant or fragment thereof.

39E. A composition comprising a mRNA comprising a construct encoding two or more heterologous polypeptide interferon effectors that suppress an interferon response, wherein the heterologous polypeptide interferon effectors comprise N, or a variant or fragment thereof, and NS1, or a variant or fragment thereof.

39F. A composition comprising a mRNA comprising a construct encoding two or more heterologous polypeptide interferon effectors that suppress an interferon response, wherein the heterologous polypeptide interferon effectors comprise N, or a variant or fragment thereof, and E3, or a variant or fragment thereof.

40. A composition comprising a mRNA comprising a construct encoding two or more heterologous polypeptide interferon effectors that suppress an interferon response, wherein the heterologous polypeptide interferon effectors are selected from the group consisting of:
(e) VP35, or a variant or fragment thereof;
(f) NS1, or a variant or fragment thereof; and
(g) E3, or a variant or fragment thereof.

41. A composition comprising a mRNA construct that enhances an interferon response, wherein the heterologous polypeptide interferon effector is PB1-F2, or a variant or fragment thereof.

42. The composition of any preceding embodiment, wherein the mRNA further comprises a construct encoding a polypeptide selected from the group consisting of: a polypeptide antigen; an antigen-binding polypeptide; an immune-modulatory polypeptide; or a therapeutic polypeptide.

43. The composition of embodiment 42, wherein the construct encodes two or more polypeptides selected from the group.

44. The composition of embodiments 41-43, wherein the coding region does not encode luciferase or green fluorescent protein (GFP).

45. A composition comprising a RNA molecule comprising from 5′ to 3′ a construct comprising one or more polynucleotide sequences encoding a polypeptide selected from the group consisting of (i) a polypeptide comprising the sequence of SEQ ID NO:17, SEQ ID NO:20; SEQ ID NO:23; or SEQ ID NO:26, (ii) a polypeptide having at least 90% identity to SEQ ID NO:17, SEQ ID NO:20, SEQ ID NO:23, or SEQ ID NO:26, or (iii) a polypeptide comprising a fragment of SEQ ID NO:17, SEQ ID NO:20, SEQ ID NO:23, or SEQ ID NO:26 that is up to 10 amino acids shorter than full-length sequence.

46. The composition of any preceding embodiment, wherein the composition comprises a non-viral delivery material.

47. The composition of embodiment 46, wherein the non-viral delivery material comprises a liposome.

48. The composition of any preceding embodiment wherein the mRNA comprises at least one N1-methylpseudouridines (N14).

49. The composition of any preceding embodiment wherein the mRNA comprises a cap 1.

50. The composition of any preceding embodiment wherein the mRNA comprises a construct encoding a polypeptide antigen and can induce an immunological response to the antigen in a subject when administered by intramuscular injection.

51. A DNA molecule encoding the RNA of embodiments 39-50.

52. A process for producing an interferon effecting or modulating RNA comprising a step of transcribing the DNA molecule of embodiment 51 to produce a RNA molecule.

53. The process of embodiment 52, wherein the transcription is in vitro.

54. The process of embodiment 53, wherein the transcription is carried out by combining the DNA, a RNA polymerase, and nucleotides in a reaction.

55. The process of embodiment 53, wherein the transcription is carried out by combining the DNA with a cellular extract.

56. The process of embodiments 52, wherein said transcription is in vivo.

57. The process of embodiment 52, further comprising a step of formulating the RNA with a non-viral delivery system.

58. A composition produced by the process of any one of embodiments 52-57.

59. A method of effecting or modulating an interferon response in a cell of a subject in need thereof, which comprises administering to said subject the composition of embodiments 39-50.

60. A method of inducing a protective or therapeutic immunological response to an antigen in a subject by administering the composition of embodiment 59 to the subject.

61. The method of embodiments 59-60, wherein the subject is human.

62. The composition of of embodiments 39-50, for use as a medicament, such as in therapy or prevention.

63. Use of the composition of embodiments 39-50, for effecting or modulating the IFN response in a subject.

64. A composition comprising a liposome comprising a mRNA encoding VP35, or a variant or fragment thereof.

65. A composition comprising a liposome comprising a mRNA encoding N, or a variant or fragment thereof.

66. The composition of embodiments 39-50 or 64-65, further comprising a nucleic acid vector encoding a polypeptide antigen or a recombinant polypeptide antigen.

### Sequences

TABLE 1
Sequence data by SEQ ID NO

| SEQ | |
|---|---|
| ID | |
| NO: | Sequence |
| 1 | ATAGGCGGCGCATGAGAGAAGCCCAGACCAATTACCTACCCAAAATGGAGAAAGTTCACGTTGACA |
| | TCGAGGAAGACAGCCCATTCCTCAGAGCTTTGCAGCGGAGCTTCCCGCAGTTTGAGGTAGAAGCCA |
| | AGCAGGTCACTGATAATGACCATGCTAATGCCAGAGCGTTTTCGCATCTGGCTTCAAAACTGATCG |
| | AAACGGAGGTGGACCCATCCGACACGATCCTTGACATTGGAAGTGCGCCCGCCCGCAGAATGTATT |
| | CTAAGCACAAGTATCATTGTATCTGTCCGATGAGATGTGCGGAAGATCCGGACAGATTGTATAAGT |
| | ATGCAACTAAGCTGAAGAAAAACTGTAAGGAAATAACTGATAAGGAATTGGACAAGAAAATGAAGG |
| | AGCTCGCCGCCGTCATGAGCGACCCTGACCTGGAAACTGAGACTATGTGCCTCCACGACGACGAGT |
| | CGTGTCGCTACGAAGGGCAAGTCGCTGTTTACCAGGATGTATACGCGGTTGACGGACCGACAAGTC |
| | TCTATCACCAAGCCAATAAGGGAGTTAGAGTCGCCTACTGGATAGGCTTTGACACCACCCCTTTTA |
| | TGTTTAAGAACTTGGCTGGAGCATATCCATCATACTCTACCAACTGGGCCGACGAAACCGTGTTAA |
| | CGGCTCGTAACATAGGCCTATGCAGCTCTGACGTTATGGAGCGGTCACGTAGAGGGATGTCCATTC |
| | TTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATT |
| | ACACATGTCGGTGTGAGACTATAGTTAGTTGCGACGGGTACGTCGTTAAAAGAATAGCTATCAGTC |
| | CAGGCCTGTATGGGAAGCCTTCAGGCTATGCTGCTACGATGCACCGCGAGGGATTCTTGTGCTGCA |
| | AAGTGACAGACACATTGAACGGGGAGAGGGTCTCTTTTCCCGTGTGCACGTATGTGCCAGCTACAT |
| | TGTGTGACCAAATGACTGGCATACTGGCAACAGATGTCAGTGCGGACGACGCGCAAAAACTGCTGG |
| | TTGGGCTCAACCAGCGTATAGTCGTCAACGGTCGCACCCAGAGAAACACCAATACCATGAAAAATT |
| | ACCTTTTGCCCGTAGTGGCCCAGGCATTTGCTAGGTGGGCAAAGGAATATAAGGAAGATCAAGAAG |
| | ATGAAAGGCCACTAGGACTACGAGATAGACAGTTAGTCATGGGGTGTTGTTGGGCTTTTAGAAGGC |
| | ACAAGATAACATCTATTTATAAGCGCCCGGATACCCAAACCATCATCAAAGTGAACAGCGATTTCC |
| | ACTCATTCGTGCTGCCCAGGATAGGCAGTAACACATTGGAGATCGGGCTGAGAACAAGAATCAGGA |
| | AAATGTTAGAGGAGCACAAGGAGCCGTCACCTCTCATTACCGCCGAGGACGTACAAGAAGCTAAGT |
| | GCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCG |
| | GCTCAGTGGAGACACCTCGTGGCTTGATAAAGGTTACCAGCTACGATGGCGAGGACAAGATCGGCT |
| | CTTACGCTGTGCTTTCTCCGCAGGCTGTACTCAAGAGTGAAAAATTATCTTGCATCCACCCTCTCG |
| | CTGAACAAGTCATAGTGATAACACACTCTGGCCGAAAAGGGCGTTATGCCGTGGAACCATACCATG |
| | GTAAAGTAGTGGTGCCAGAGGGACATGCAATACCCGTCCAGGACTTTCAAGCTCTGAGTGAAAGTG |
| | CCACCATTGTGTACAACGAACGTGAGTTCGTAAACAGGTACCTGCACCATATTGCCACACATGGAG |
| | GAGCGCTGAACACTGATGAAGAATATTACAAAACTGTCAAGCCCAGCGAGCACGACGGCGAATACC |
| | TGTACGACATCGACAGGAAACAGTGCGTCAAGAAAGAACTAGTCACTGGGCTAGGGCTCACAGGCG |
| | AGCTGGTGGATCCTCCCTTCCATGAATTCGCCTACGAGAGTCTGAGAACACGACCAGCCGCTCCTT |
| | ACCAAGTACCAACCATAGGGGTGTATGGCGTGCCAGGATCAGGCAAGTCTGGCATCATTAAAAGCG |
| | CAGTCACCAAAAAAGATCTAGTGGTGAGCGCCAAGAAAGAAAACTGTGCAGAAATTATAAGGGACG |
| | TCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGC |
| | TCATAGCCATTATAAGACCTAAAAAGGCAGTGCTCTGCGGGGATCCCAAACAGTGCGGTTTTTTTA |
| | ACATGATGTGCCTGAAAGTGCATTTTAACCACGAGATTTGCACACAAGTCTTCCACAAAAGCATCT |
| | CTCGCCGTTGCACTAAATCTGTGACTTCGGTCGTCTCAACCTTGTTTTACGACAAAAAAATGAGAA |
| | CGACGAATCCGAAAGAGACTAAGATTGTGATTGACACTACCGGCAGTACCAAACCTAAGCAGGACG |
| | ATCTCATTCTCACTTGTTTCAGAGGGTGGGTGAAGCAGTTGCAAATAGATTACAAAGGCAACGAAA |
| | TAATGACGGCAGCTGCCTCTCAAGGGCTGACCCGTAAAGGTGTGTATGCCGTTCGGTACAAGGTGA |
| | ATGAAAATCCTCTGTACGCACCCACCTCAGAACATGTGAACGTCCTACTGACCCGCACGGAGGACC |
| | GCATCGTGTGGAAAACACTAGCCGGCGACCCATGGATAAAAACACTGACTGCCAAGTACCCTGGGA |
| | ATTTCACTGCCACGATAGAGGAGTGGCAAGCAGAGCATGATGCCATCATGAGGCACATCTTGGAGA |
| | GACCGGACCCTACCGACGTCTTCCAGAATAAGGCAAACGTGTGTTGGGCCAAGGCTTTAGTGCCGG |
| | TGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGG |
| | ACTCCGGTCTATTTTCTGCACCCACTGTTCCGTTATCCATTAGGAATAATCACTGGGATAACTCCC |
| | CGTCGCCTAACATGTACGGGCTGAATAAAGAAGTGGTCCGTCAGCTCTCTCGCAGGTACCCACAAC |
| | TGCCTCGGGCAGTTGCCACTGGAAGAGTCTATGACATGAACACTGGTACACTGCGCAATTATGATC |
| | CGCGCATAAACCTAGTACCTGTAAACAGAAGACTGCCTCATGCTTTAGTCCTCCACCATAATGAAC |
| | ACCCACAGAGTGACTTTTCTTCATTCGTCAGCAAATTGAAGGGCAGAACTGTCCTGGTGGTCGGGG |
| | AAAAGTTGTCCGTCCCAGGCAAAATGGTTGACTGGTTGTCAGACCGGCCTGAGGCTACCTTCAGAG |
| | CTCGGCTGGATTTAGGCATCCCAGGTGATGTGCCCAAATATGACATAATATTTGTTAATGTGAGGA |
| | CCCCATATAAATACCATCACTATCAGCAGTGTGAAGACCATGCCATTAAGCTTAGCATGTTGACCA |
| | AGAAAGCTTGTCTGCATCTGAATCCCGGCGGAACCTGTGTCAGCATAGGTTATGGTTACGCTGACA |
| | GGGCCAGCGAAAGCATCATTGGTGCTATAGCGCGGCAGTTCAAGTTTTCCCGGGTATGCAAACCGA |
| | AATCCTCACTTGAAGAGACGGAAGTTCTGTTTGTATTCATTGGGTACGATCGCAAGGCCCGTACGC |
| | ACAATCCTTACAAGCTTTCATCAACCTTGACCAACATTTATACAGGTTCCAGACTCCACGAAGCCG |
| | GATGTGCACCCTCATATCATGTGGTGCGAGGGGATATTGCCACGGCCACCGAAGGAGTGATTATAA |
| | ATGCTGCTAACAGCAAAGGACAACCTGGCGGAGGGGTGTGCGGAGCGCTGTATAAGAAATTCCCGG |
| | AAAGCTTCGATTTACAGCCGATCGAAGTAGGAAAAGCGCGACTGGTCAAAGGTGCAGCTAAACATA |
| | TCATTCATGCCGTAGGACCAAACTTCAACAAAGTTTCGGAGGTTGAAGGTGACAAACAGTTGGCAG |
| | AGGCTTATGAGTCCATCGCTAAGATTGTCAACGATAACAATTACAAGTCAGTAGCGATTCCACTGT |
| | TGTCCACCGGCATCTTTTCCGGGAACAAAGATCGACTAACCCAATCATTGAACCATTTGCTGACAG |
| | CTTTAGACACCACTGATGCAGATGTAGCCATATACTGCAGGGACAAGAAATGGGAAATGACTCTCA |
| | AGGAAGCAGTGGCTAGGAGAGAAGCAGTGGAGGAGATATGCATATCCGACGACTCTTCAGTGACAG |
| | AACCTGATGCAGAGCTGGTGAGGGTGCATCCGAAGAGTTCTTTGGCTGGAAGGAAGGGCTACAGCA |
| | CAAGCGATGGCAAAACTTTCTCATATTTGGAAGGGACCAAGTTTCACCAGGCGGCCAAGGATATAG |
| | CAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGAGTCGGAAGCCTCCACACCACCTA |
| | GCACGCTGCCTTGCTTGTGCATCCATGCCATGACTCCAGAAAGAGTACAGCGCCTAAAAGCCTCAC |
| | GTCCAGAACAAATTACTGTGTGCTCATCCTTTCCATTGCCGAAGTATAGAATCACTGGTGTGCAGA |
| | AGATCCAATGCTCCCAGCCTATATTGTTCTCACCGAAAGTGCCTGCGTATATTCATCCAAGGAAGT |
| | ATCTCGTGGAAACACCACCGGTAGACGAGACTCCGGAGCCATCGGCAGAGAACCAATCCACAGAGG |
| | GGACACCTGAACAACCACCACTTATAACCGAGGATGAGACCAGGACTAGAACGCCTGAGCCGATCA |
| | TCATCGAAGAGGAAGAAGAGGATAGCATAAGTTTGCTGTCAGATGGCCCGACCCACCAGGTGCTGC |
| | AAGTCGAGGCAGACATTCACGGGCCGCCCTCTGTATCTAGCTCATCCTGGTCCATTCCTCATGCAT |
| | CCGACTTTGATGTGGACAGTTTATCCATACTTGACACCCTGGAGGGAGCTAGCGTGACCAGCGGGG |
| | CAACGTCAGCCGAGACTAACTCTTACTTCGCAAAGAGTATGGAGTTTCTGGCGCGACCGGTGCCTG |
| | CGCCTCGAACAGTATTCAGGAACCCTCCACATCCCGCTCCGCGCACAAGAACACCGTCACTTGCAC |
| | CCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGG |
| | TCTCCAACCCGCCAGGCGTAAATAGGGTGATTACAAGAGAGGAGTTTGAGGCGTTCGTAGCACAAC |
| | AACAATGACGGTTTGATGCGGGTGCATACATCTTTTCCTCCGACACCGGTCAAGGGCATTTACAAC |
| | AAAAATCAGTAAGGCAAACGGTGCTATCCGAAGTGGTGTTGGAGAGGACCGAATTGGAGATTTCGT |
| | ATGCCCCGCGCCTCGACCAAGAAAAAGAAGAATTACTACGCAAGAAATTACAGTTAAATCCCACAC |
| | CTGCTAACAGAAGCAGATACCAGTCCAGGAAGGTGGAGAACATGAAAGCCATAACAGCTAGACGTA |
| | TTCTGCAAGGCCTAGGGCATTATTTGAAGGCAGAAGGAAAAGTGGAGTGCTACCGAACCCTGCATC |
| | CTGTTCCTTTGTATTCATCTAGTGTGAACCGTGCCTTTTCAAGCCCCAAGGTCGCAGTGGAAGCCT |
| | GTAACGCCATGTTGAAAGAGAACTTTCCGACTGTGGCTTCTTACTGTATTATTCCAGAGTACGATG |
| | CCTATTTGGACATGGTTGACGGAGCTTCATGCTGCTTAGACACTGCCAGTTTTTGCCCTGCAAAGC |
| | TGCGCAGCTTTCCAAAGAAACACTCCTATTTGGAACCCACAATACGATCGGCAGTGCCTTCAGCGA |
| | TCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATA |
| | ATGAATATTGGGAAACGTTTAAAGAAAACCCCATCAGGCTTACTGAAGAAAACGTGGTAAATTACA |
| | TTACCAAATTAAAAGGACCAAAAGCTGCTGCTCTTTTTGCGAAGACACATAATTTGAATATGTTGC |
| | AGGACATACCAATGGACAGGTTTGTAATGGACTTAAAGAGAGACGTGAAAGTGACTCCAGGAACAA |
| | AACATACTGAAGAACGGCCCAAGGTACAGGTGATCCAGGCTGCCGATCCGCTAGCAACAGCGTATC |
| | TGTGCGGAATCCACCGAGAGCTGGTTAGGAGATTAAATGCGGTCCTGCTTCCGAACATTCATACAC |
| | TGTTTGATATGTCGGCTGAAGACTTTGACGCTATTATAGCCGAGCACTTCCAGCCTGGGGATTGTG |
| | TTCTGGAAACTGACATCGCGTCGTTTGATAAAAGTGAGGACGACGCCATGGCTCTGACCGCGTTAA |
| | TGATTCTGGAAGACTTAGGTGTGGACGCAGAGCTGTTGACGCTGATTGAGGCGGCTTTCGGCGAAA |
| | TTTCATCAATACATTTGCCCACTAAAACTAAATTTAAATTCGGAGCCATGATGAAATCTGGAATGT |
| | TCCTCACACTGTTTGTGAACACAGTCATTAACATTGTAATCGCAAGCAGAGTGTTGAGAGAACGGC |
| | TAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGG |
| | GCGAGAAAGCGCCTTATTTCTGTGGAGGGTTTATTTTGTGTGACTCCGTGACCGGCACAGCGTGCC |
| | GTGTGGCAGACCCCCTAAAAAGGCTGTTTAAGCTTGGCAAACCTCTGGCAGCAGACGATGAACATG |
| | ATGATGACAGGAGAAGGGCATTGCATGAAGAGTCAACACGCTGGAACCGAGTGGGTATTCTTTCAG |
| | AGCTGTGCAAGGCAGTAGAATCAAGGTATGAAACCGTAGGAACTTCCATCATAGTTATGGCCATGA |
| | CTACTCTAGCTAGCAGTGTTAAATCATTCAGCTACCTGAGAGGGGCCCCTATAACTCTCTACGGCT |
| | AACCTGAATGGACTACGACATAGTCTAGTCCGCCAAG |
| 2 | AUAGGCGGCGCAUGAGAGAAGCCCAGACCAAUUACCUACCCAAAAUGGAGAAAGUUCACGUUGACA |
| | UCGAGGAAGACAGCCCAUUCCUCAGAGCUUUGCAGCGGAGCUUCCCGCAGUUUGAGGUAGAAGCCA |
| | AGCAGGUCACUGAUAAUGACCAUGCUAAUGCCAGAGCGUUUUCGCAUCUGGCUUCAAAACUGAUCG |
| | AAACGGAGGUGGACCCAUCCGACACGAUCCUUGACAUUGGAAGUGCGCCCGCCCGCAGAAUGUAUU |
| | CUAAGCACAAGUAUCAUUGUAUCUGUCCGAUGAGAUGUGCGGAAGAUCCGGACAGAUUGUAUAAGU |
| | AUGCAACUAAGCUGAAGAAAAACUGUAAGGAAAUAACUGAUAAGGAAUUGGACAAGAAAAUGAAGG |
| | AGCUCGCCGCCGUCAUGAGCGACCCUGACCUGGAAACUGAGACUAUGUGCCUCCACGACGACGAGU |
| | CGUGUCGCUACGAAGGGCAAGUCGCUGUUUACCAGGAUGUAUACGCGGUUGACGGACCGACAAGUC |
| | UCUAUCACCAAGCCAAUAAGGGAGUUAGAGUCGCCUACUGGAUAGGCUUUGACACCACCCCUUUUA |
| | UGUUUAAGAACUUGGCUGGAGCAUAUCCAUCAUACUCUACCAACUGGGCCGACGAAACCGUGUUAA |
| | CGGCUCGUAACAUAGGCCUAUGCAGCUCUGACGUUAUGGAGCGGUCACGUAGAGGGAUGUCCAUUC |
| | UUAGAAAGAAGUAUUUGAAACCAUCCAACAAUGUUCUAUUCUCUGUUGGCUCGACCAUCUACCACG |
| | AGAAGAGGGACUUACUGAGGAGCUGGCACCUGCCGUCUGUAUUUCACUUACGUGGCAAGCAAAAUU |
| | ACACAUGUCGGUGUGAGACUAUAGUUAGUUGCGACGGGUACGUCGUUAAAAGAAUAGCUAUCAGUC |
| | CAGGCCUGUAUGGGAAGCCUUCAGGCUAUGCUGCUACGAUGCACCGCGAGGGAUUCUUGUGCUGCA |
| | AAGUGACAGACACAUUGAACGGGGAGAGGGUCUCUUUUCCCGUGUGCACGUAUGUGCCAGCUACAU |
| | UGUGUGACCAAAUGACUGGCAUACUGGCAACAGAUGUCAGUGCGGACGACGCGCAAAAACUGCUGG |
| | UUGGGCUCAACCAGCGUAUAGUCGUCAACGGUCGCACCCAGAGAAACACCAAUACCAUGAAAAAUU |
| | ACCUUUUGCCCGUAGUGGCCCAGGCAUUUGCUAGGUGGGCAAAGGAAUAUAAGGAAGAUCAAGAAG |
| | AUGAAAGGCCACUAGGACUACGAGAUAGACAGUUAGUCAUGGGGUGUUGUUGGGCUUUUAGAAGGC |
| | ACAAGAUAACAUCUAUUUAUAAGCGCCCGGAUACCCAAACCAUCAUCAAAGUGAACAGCGAUUUCC |
| | ACUCAUUCGUGCUGCCCAGGAUAGGCAGUAACACAUUGGAGAUCGGGCUGAGAACAAGAAUCAGGA |
| | AAAUGUUAGAGGAGCACAAGGAGCCGUCACCUCUCAUUACCGCCGAGGACGUACAAGAAGCUAAGU |
| | GCGCAGCCGAUGAGGCUAAGGAGGUGCGUGAAGCCGAGGAGUUGCGCGCAGCUCUACCACCUUUGG |
| | CAGCUGAUGUUGAGGAGCCCACUCUGGAAGCCGAUGUCGACUUGAUGUUACAAGAGGCUGGGGCCG |
| | GCUCAGUGGAGACACCUCGUGGCUUGAUAAAGGUUACCAGCUACGAUGGCGAGGACAAGAUCGGCU |
| | CUUACGCUGUGCUUUCUCCGCAGGCUGUACUCAAGAGUGAAAAAUUAUCUUGCAUCCACCCUCUCG |
| | CUGAACAAGUCAUAGUGAUAACACACUCUGGCCGAAAAGGGCGUUAUGCCGUGGAACCAUACCAUG |
| | GUAAAGUAGUGGUGCCAGAGGGACAUGCAAUACCCGUCCAGGACUUUCAAGCUCUGAGUGAAAGUG |
| | CCACCAUUGUGUACAACGAACGUGAGUUCGUAAACAGGUACCUGCACCAUAUUGCCACACAUGGAG |
| | GAGCGCUGAACACUGAUGAAGAAUAUUACAAAACUGUCAAGCCCAGCGAGCACGACGGCGAAUACC |
| | UGUACGACAUCGACAGGAAACAGUGCGUCAAGAAAGAACUAGUCACUGGGCUAGGGCUCACAGGCG |
| | AGCUGGUGGAUCCUCCCUUCCAUGAAUUCGCCUACGAGAGUCUGAGAACACGACCAGCCGCUCCUU |
| | ACCAAGUACCAACCAUAGGGGUGUAUGGCGUGCCAGGAUCAGGCAAGUCUGGCAUCAUUAAAAGCG |
| | CAGUCACCAAAAAAGAUCUAGUGGUGAGCGCCAAGAAAGAAAACUGUGCAGAAAUUAUAAGGGACG |
| | UCAAGAAAAUGAAAGGGCUGGACGUCAAUGCCAGAACUGUGGACUCAGUGCUCUUGAAUGGAUGCA |
| | AACACCCCGUAGAGACCCUGUAUAUUGACGAAGCUUUUGCUUGUCAUGCAGGUACUCUCAGAGCGC |
| | UCAUAGCCAUUAUAAGACCUAAAAAGGCAGUGCUCUGCGGGGAUCCCAAACAGUGCGGUUUUUUUA |
| | ACAUGAUGUGCCUGAAAGUGCAUUUUAACCACGAGAUUUGCACACAAGUCUUCCACAAAAGCAUCU |
| | CUCGCCGUUGCACUAAAUCUGUGACUUCGGUCGUCUCAACCUUGUUUUACGACAAAAAAAUGAGAA |
| | CGACGAAUCCGAAAGAGACUAAGAUUGUGAUUGACACUACCGGCAGUACCAAACCUAAGCAGGACG |
| | AUCUCAUUCUCACUUGUUUCAGAGGGUGGGUGAAGCAGUUGCAAAUAGAUUACAAAGGCAACGAAA |
| | UAAUGACGGCAGCUGCCUCUCAAGGGCUGACCCGUAAAGGUGUGUAUGCCGUUCGGUACAAGGUGA |
| | AUGAAAAUCCUCUGUACGCACCCACCUCAGAACAUGUGAACGUCCUACUGACCCGCACGGAGGACC |
| | GCAUCGUGUGGAAAACACUAGCCGGCGACCCAUGGAUAAAAACACUGACUGCCAAGUACCCUGGGA |
| | AUUUCACUGCCACGAUAGAGGAGUGGCAAGCAGAGCAUGAUGCCAUCAUGAGGCACAUCUUGGAGA |
| | GACCGGACCCUACCGACGUCUUCCAGAAUAAGGCAAACGUGUGUUGGGCCAAGGCUUUAGUGCCGG |
| | UGCUGAAGACCGCUGGCAUAGACAUGACCACUGAACAAUGGAACACUGUGGAUUAUUUUGAAACGG |
| | ACAAAGCUCACUCAGCAGAGAUAGUAUUGAACCAACUAUGCGUGAGGUUCUUUGGACUCGAUCUGG |
| | ACUCCGGUCUAUUUUCUGCACCCACUGUUCCGUUAUCCAUUAGGAAUAAUCACUGGGAUAACUCCC |
| | CGUCGCCUAACAUGUACGGGCUGAAUAAAGAAGUGGUCCGUCAGCUCUCUCGCAGGUACCCACAAC |
| | UGCCUCGGGCAGUUGCCACUGGAAGAGUCUAUGACAUGAACACUGGUACACUGCGCAAUUAUGAUC |
| | CGCGCAUAAACCUAGUACCUGUAAACAGAAGACUGCCUCAUGCUUUAGUCCUCCACCAUAAUGAAC |
| | ACCCACAGAGUGACUUUUCUUCAUUCGUCAGCAAAUUGAAGGGCAGAACUGUCCUGGUGGUCGGGG |
| | AAAAGUUGUCCGUCCCAGGCAAAAUGGUUGACUGGUUGUCAGACCGGCCUGAGGCUACCUUCAGAG |
| | CUCGGCUGGAUUUAGGCAUCCCAGGUGAUGUGCCCAAAUAUGACAUAAUAUUUGUUAAUGUGAGGA |
| | CCCCAUAUAAAUACCAUCACUAUCAGCAGUGUGAAGACCAUGCCAUUAAGCUUAGCAUGUUGACCA |
| | AGAAAGCUUGUCUGCAUCUGAAUCCCGGCGGAACCUGUGUCAGCAUAGGUUAUGGUUACGCUGACA |
| | GGGCCAGCGAAAGCAUCAUUGGUGCUAUAGCGCGGCAGUUCAAGUUUUCCCGGGUAUGCAAACCGA |
| | AAUCCUCACUUGAAGAGACGGAAGUUCUGUUUGUAUUCAUUGGGUACGAUCGCAAGGCCCGUACGC |
| | ACAAUCCUUACAAGCUUUCAUCAACCUUGACCAACAUUUAUACAGGUUCCAGACUCCACGAAGCCG |
| | GAUGUGCACCCUCAUAUCAUGUGGUGCGAGGGGAUAUUGCCACGGCCACCGAAGGAGUGAUUAUAA |
| | AUGCUGCUAACAGCAAAGGACAACCUGGCGGAGGGGUGUGCGGAGCGCUGUAUAAGAAAUUCCCGG |
| | AAAGCUUCGAUUUACAGCCGAUCGAAGUAGGAAAAGCGCGACUGGUCAAAGGUGCAGCUAAACAUA |
| | UCAUUCAUGCCGUAGGACCAAACUUCAACAAAGUUUCGGAGGUUGAAGGUGACAAACAGUUGGCAG |
| | AGGCUUAUGAGUCCAUCGCUAAGAUUGUCAACGAUAACAAUUACAAGUCAGUAGCGAUUCCACUGU |
| | UGUCCACCGGCAUCUUUUCCGGGAACAAAGAUCGACUAACCCAAUCAUUGAACCAUUUGCUGACAG |
| | CUUUAGACACCACUGAUGCAGAUGUAGCCAUAUACUGCAGGGACAAGAAAUGGGAAAUGACUCUCA |
| | AGGAAGCAGUGGCUAGGAGAGAAGCAGUGGAGGAGAUAUGCAUAUCCGACGACUCUUCAGUGACAG |
| | AACCUGAUGCAGAGCUGGUGAGGGUGCAUCCGAAGAGUUCUUUGGCUGGAAGGAAGGGCUACAGCA |
| | CAAGCGAUGGCAAAACUUUCUCAUAUUUGGAAGGGACCAAGUUUCACCAGGCGGCCAAGGAUAUAG |
| | CAGAAAUUAAUGCCAUGUGGCCCGUUGCAACGGAGGCCAAUGAGCAGGUAUGCAUGUAUAUCCUCG |
| | GAGAAAGCAUGAGCAGUAUUAGGUCGAAAUGCCCCGUCGAAGAGUCGGAAGCCUCCACACCACCUA |
| | GCACGCUGCCUUGCUUGUGCAUCCAUGCCAUGACUCCAGAAAGAGUACAGCGCCUAAAAGCCUCAC |
| | GUCCAGAACAAAUUACUGUGUGCUCAUCCUUUCCAUUGCCGAAGUAUAGAAUCACUGGUGUGCAGA |
| | AGAUCCAAUGCUCCCAGCCUAUAUUGUUCUCACCGAAAGUGCCUGCGUAUAUUCAUCCAAGGAAGU |
| | AUCUCGUGGAAACACCACCGGUAGACGAGACUCCGGAGCCAUCGGCAGAGAACCAAUCCACAGAGG |
| | GGACACCUGAACAACCACCACUUAUAACCGAGGAUGAGACCAGGACUAGAACGCCUGAGCCGAUCA |
| | UCAUCGAAGAGGAAGAAGAGGAUAGCAUAAGUUUGCUGUCAGAUGGCCCGACCCACCAGGUGCUGC |
| | AAGUCGAGGCAGACAUUCACGGGCCGCCCUCUGUAUCUAGCUCAUCCUGGUCCAUUCCUCAUGCAU |
| | CCGACUUUGAUGUGGACAGUUUAUCCAUACUUGACACCCUGGAGGGAGCUAGCGUGACCAGCGGGG |
| | CAACGUCAGCCGAGACUAACUCUUACUUCGCAAAGAGUAUGGAGUUUCUGGCGCGACCGGUGCCUG |
| | CGCCUCGAACAGUAUUCAGGAACCCUCCACAUCCCGCUCCGCGCACAAGAACACCGUCACUUGCAC |
| | CCAGCAGGGCCUGCUCGAGAACCAGCCUAGUUUCCACCCCGCCAGGCGUGAAUAGGGUGAUCACUA |
| | GAGAGGAGCUCGAGGCGCUUACCCCGUCACGCACUCCUAGCAGGUCGGUCUCGAGAACCAGCCUGG |
| | UCUCCAACCCGCCAGGCGUAAAUAGGGUGAUUACAAGAGAGGAGUUUGAGGCGUUCGUAGCACAAC |
| | AACAAUGACGGUUUGAUGCGGGUGCAUACAUCUUUUCCUCCGACACCGGUCAAGGGCAUUUACAAC |
| | AAAAAUCAGUAAGGCAAACGGUGCUAUCCGAAGUGGUGUUGGAGAGGACCGAAUUGGAGAUUUCGU |
| | AUGCCCCGCGCCUCGACCAAGAAAAAGAAGAAUUACUACGCAAGAAAUUACAGUUAAAUCCCACAC |
| | CUGCUAACAGAAGCAGAUACCAGUCCAGGAAGGUGGAGAACAUGAAAGCCAUAACAGCUAGACGUA |
| | UUCUGCAAGGCCUAGGGCAUUAUUUGAAGGCAGAAGGAAAAGUGGAGUGCUACCGAACCCUGCAUC |
| | CUGUUCCUUUGUAUUCAUCUAGUGUGAACCGUGCCUUUUCAAGCCCCAAGGUCGCAGUGGAAGCCU |
| | GUAACGCCAUGUUGAAAGAGAACUUUCCGACUGUGGCUUCUUACUGUAUUAUUCCAGAGUACGAUG |
| | CCUAUUUGGACAUGGUUGACGGAGCUUCAUGCUGCUUAGACACUGCCAGUUUUUGCCCUGCAAAGC |
| | UGCGCAGCUUUCCAAAGAAACACUCCUAUUUGGAACCCACAAUACGAUCGGCAGUGCCUUCAGCGA |
| | UCCAGAACACGCUCCAGAACGUCCUGGCAGCUGCCACAAAAAGAAAUUGCAAUGUCACGCAAAUGA |
| | GAGAAUUGCCCGUAUUGGAUUCGGCGGCCUUUAAUGUGGAAUGCUUCAAGAAAUAUGCGUGUAAUA |
| | AUGAAUAUUGGGAAACGUUUAAAGAAAACCCCAUCAGGCUUACUGAAGAAAACGUGGUAAAUUACA |
| | UUACCAAAUUAAAAGGACCAAAAGCUGCUGCUCUUUUUGCGAAGACACAUAAUUUGAAUAUGUUGC |
| | AGGACAUACCAAUGGACAGGUUUGUAAUGGACUUAAAGAGAGACGUGAAAGUGACUCCAGGAACAA |
| | AACAUACUGAAGAACGGCCCAAGGUACAGGUGAUCCAGGCUGCCGAUCCGCUAGCAACAGCGUAUC |
| | UGUGCGGAAUCCACCGAGAGCUGGUUAGGAGAUUAAAUGCGGUCCUGCUUCCGAACAUUCAUACAC |
| | UGUUUGAUAUGUCGGCUGAAGACUUUGACGCUAUUAUAGCCGAGCACUUCCAGCCUGGGGAUUGUG |
| | UUCUGGAAACUGACAUCGCGUCGUUUGAUAAAAGUGAGGACGACGCCAUGGCUCUGACCGCGUUAA |
| | UGAUUCUGGAAGACUUAGGUGUGGACGCAGAGCUGUUGACGCUGAUUGAGGCGGCUUUCGGCGAAA |
| | UUUCAUCAAUACAUUUGCCCACUAAAACUAAAUUUAAAUUCGGAGCCAUGAUGAAAUCUGGAAUGU |
| | UCCUCACACUGUUUGUGAACACAGUCAUUAACAUUGUAAUCGCAAGCAGAGUGUUGAGAGAACGGC |
| | UAACCGGAUCACCAUGUGCAGCAUUCAUUGGAGAUGACAAUAUCGUGAAAGGAGUCAAAUCGGACA |
| | AAUUAAUGGCAGACAGGUGCGCCACCUGGUUGAAUAUGGAAGUCAAGAUUAUAGAUGCUGUGGUGG |
| | GCGAGAAAGCGCCUUAUUUCUGUGGAGGGUUUAUUUUGUGUGACUCCGUGACCGGCACAGCGUGCC |
| | GUGUGGCAGACCCCCUAAAAAGGCUGUUUAAGCUUGGCAAACCUCUGGCAGCAGACGAUGAACAUG |
| | AUGAUGACAGGAGAAGGGCAUUGCAUGAAGAGUCAACACGCUGGAACCGAGUGGGUAUUCUUUCAG |
| | AGCUGUGCAAGGCAGUAGAAUCAAGGUAUGAAACCGUAGGAACUUCCAUCAUAGUUAUGGCCAUGA |
| | CUACUCUAGCUAGCAGUGUUAAAUCAUUCAGCUACCUGAGAGGGGCCCCUAUAACUCUCUACGGCU |
| | AACCUGAAUGGACUACGACAUAGUCUAGUCCGCCAAG |
| 3 | TCTAGAGGCCCCTATAACTCTCTACGGCTAACCTGAATGGACTACGACATAGTCTAGTCCGCCAAG |
| 4 | UCUAGAGGCCCCUAUAACUCUCUACGGCUAACCUGAAUGGACUACGACAUAGUCUAGUCCGCCAAG |
| 5 | TCTAGACGGCGCGCCCACCCAGCGGCCGCATACAGCAGCAATTGGCAAGCTGCTTACATAGAACTC |
| | GCGGCGATTGGCATGCCGCCTTAAAATTTTTATTTTATTTTTCTTTTCTTTTCCGAATCGGATTTT |
| | GTTTTTAATATTTCAAAAAAAAAAAAAAAAAAAAAAAAAAAAAAAAAAAAAAAAGAAGAGCGTTTA |
| | AACACGTGATATCTGGCCTCATGGGCCTTCCTTTCACTGCCCGCTTTCCAGTCGGGAAACCTGTCG |
| | TGCCAGCTGCATTAACATGGTCATAGCTGTTTCCTTGCGTATTGGGCGCTCTCCGCTTCCTCGCTC |
| | ACTGACTCGCTGCGCTCGGTCGTTCGGGTAAAGCCTGGGGTGCCTAATGAGCAAAAGGCCAGCAAA |
| | AGGCCAGGAACCGTAAAAAGGCCGCGTTGCTGGCGTTTTTCCATAGGCTCCGCCCCCCTGACGAGC |
| | ATCACAAAAATCGACGCTCAAGTCAGAGGTGGCGAAACCCGACAGGACTATAAAGATACCAGGCGT |
| | TTCCCCCTGGAAGCTCCCTCGTGCGCTCTCCTGTTCCGACCCTGCCGCTTACCGGATACCTGTCCG |
| | CCTTTCTCCCTTCGGGAAGCGTGGCGCTTTCTCATAGCTCACGCTGTAGGTATCTCAGTTCGGTGT |
| | AGGTCGTTCGCTCCAAGCTGGGCTGTGTGCACGAACCCCCCGTTCAGCCCGACCGCTGCGCCTTAT |
| | CCGGTAACTATCGTCTTGAGTCCAACCCGGTAAGACACGACTTATCGCCACTGGCAGCAGCCACTG |
| | GTAACAGGATTAGCAGAGCGAGGTATGTAGGCGGTGCTACAGAGTTCTTGAAGTGGTGGCCTAACT |
| | ACGGCTACACTAGAAGAACAGTATTTGGTATCTGCGCTCTGCTGAAGCCAGTTACCTTCGGAAAAA |
| | GAGTTGGTAGCTCTTGATCCGGCAAACAAACCACCGCTGGTAGCGGTGGTTTTTTTGTTTGCAAGC |
| | AGCAGATTACGCGCAGAAAAAAAGGATCTCAAGAAGATCCTTTGATCTTTTCTACGGGGTCTGACG |
| | CTCAGTGGAACGAAAACTCACGTTAAGGGATTTTGGTCATGAATACACGGTGCCTGACTGCGTTAG |
| | CAATTTAACTGTGATAAACTACCGCATTAAAGCTTATCGATGATAAGCTGTCAAACATGAGAATTC |
| | TTAGAAAAACTCATCGAGCATCAAATGAAACTGCAATTTATTCATATCAGGATTATCAATACCATA |
| | TTTTTGAAAAAGCCGTTTCTGTAATGAAGGAGAAAACTCACCGAGGCAGTTCCATAGGATGGCAAG |
| | ATCCTGGTATCGGTCTGCGATTCCGACTCGTCCAACATCAATACAACCTATTAATTTCCCCTCGTC |
| | AAAAATAAGGTTATCAAGTGAGAAATCACCATGAGTGACGACTGAATCCGGTGAGAATGGCAAAAG |
| | CTTATGCATTTCTTTCCAGACTTGTTCAACAGGCCAGCCATTACGCTCGTCATCAAAATCACTCGC |
| | ATCAACCAAACCGTTATTCATTCGTGATTGCGCCTGAGCGAGACGAAATACGCGATCGCTGTTAAA |
| | AGGACAATTACAAACAGGAATCGAATGCAACCGGCGCAGGAACACTGCCAGCGCATCAACAATATT |
| | TTCACCTGAATCAGGATATTCTTCTAATACCTGGAATGCTGTTTTCCCGGGGATCGCAGTGGTGAG |
| | TAACCATGCATCATCAGGAGTACGGATAAAATGCTTGATGGTCGGAAGAGGCATAAATTCCGTCAG |
| | CCAGTTTAGTCTGACCATCTCATCTGTAACATCATTGGCAACGCTACCTTTGCCATGTTTCAGAAA |
| | CAACTCTGGCGCATCGGGCTTCCCATACAATCGATAGATTGTCGCACCTGATTGCCCGACATTATC |
| | GCGAGCCCATTTATACCCATATAAATCAGCATCCATGTTGGAATTTAATCGCGGCCTCGAGCAAGA |
| | CGTTTCCCGTTGAATATGGCTCATAACACCCCTTGTATTACTGTTTATGTAAGCAGACAGTTTTAT |
| | TGTTCATGAGCGGATACATATTTGAATGTATTTAGAAAAATAAACAAATAGGGGTTCCGCGCACAT |
| | TTCCCCGAAAAGTGCCACCTAAATTGTAAGCGTTAATATTTTGTTAAAATTCGCGTTAAATTTTTG |
| | TTAAATCAGCTCATTTTTTAACCAATAGGCCGAAATCGGCAAAATCCCTTATAAATCAAAAGAATA |
| | GACCGAGATAGGGTTGAGTGGCCGCTACAGGGCGCTCCCATTCGCCATTCAGGCTGCGCAACTGTT |
| | GGGAAGGGCGTTTCGGTGCGGGCCTCTTCGCTATTACGCCAGCTGGCGAAAGGGGGATGTGCTGCA |
| | AGGCGATTAAGTTGGGTAACGCCAGGGTTTTCCCAGTCACACGCGTAATACGACTCACTATAG |
| 6 | UCUAGACGGCGCGCCCACCCAGCGGCCGCAUACAGCAGCAAUUGGCAAGCUGCUUACAUAGAACUC |
| | GCGGCGAUUGGCAUGCCGCCUUAAAAUUUUUAUUUUAUUUUUCUUUUCUUUUCCGAAUCGGAUUUU |
| | GUUUUUAAUAUUUCAAAAAAAAAAAAAAAAAAAAAAAAAAAAAAAAAAAAAAAAGAAGAGCGUUUA |
| | AACACGUGAUAUCUGGCCUCAUGGGCCUUCCUUUCACUGCCCGCUUUCCAGUCGGGAAACCUGUCG |
| | UGCCAGCUGCAUUAACAUGGUCAUAGCUGUUUCCUUGCGUAUUGGGCGCUCUCCGCUUCCUCGCUC |
| | ACUGACUCGCUGCGCUCGGUCGUUCGGGUAAAGCCUGGGGUGCCUAAUGAGCAAAAGGCCAGCAAA |
| | AGGCCAGGAACCGUAAAAAGGCCGCGUUGCUGGCGUUUUUCCAUAGGCUCCGCCCCCCUGACGAGC |
| | AUCACAAAAAUCGACGCUCAAGUCAGAGGUGGCGAAACCCGACAGGACUAUAAAGAUACCAGGCGU |
| | UUCCCCCUGGAAGCUCCCUCGUGCGCUCUCCUGUUCCGACCCUGCCGCUUACCGGAUACCUGUCCG |
| | CCUUUCUCCCUUCGGGAAGCGUGGCGCUUUCUCAUAGCUCACGCUGUAGGUAUCUCAGUUCGGUGU |
| | AGGUCGUUCGCUCCAAGCUGGGCUGUGUGCACGAACCCCCCGUUCAGCCCGACCGCUGCGCCUUAU |
| | CCGGUAACUAUCGUCUUGAGUCCAACCCGGUAAGACACGACUUAUCGCCACUGGCAGCAGCCACUG |
| | GUAACAGGAUUAGCAGAGCGAGGUAUGUAGGCGGUGCUACAGAGUUCUUGAAGUGGUGGCCUAACU |
| | ACGGCUACACUAGAAGAACAGUAUUUGGUAUCUGCGCUCUGCUGAAGCCAGUUACCUUCGGAAAAA |
| | GAGUUGGUAGCUCUUGAUCCGGCAAACAAACCACCGCUGGUAGCGGUGGUUUUUUUGUUUGCAAGC |
| | AGCAGAUUACGCGCAGAAAAAAAGGAUCUCAAGAAGAUCCUUUGAUCUUUUCUACGGGGUCUGACG |
| | CUCAGUGGAACGAAAACUCACGUUAAGGGAUUUUGGUCAUGAAUACACGGUGCCUGACUGCGUUAG |
| | CAAUUUAACUGUGAUAAACUACCGCAUUAAAGCUUAUCGAUGAUAAGCUGUCAAACAUGAGAAUUC |
| | UUAGAAAAACUCAUCGAGCAUCAAAUGAAACUGCAAUUUAUUCAUAUCAGGAUUAUCAAUACCAUA |
| | UUUUUGAAAAAGCCGUUUCUGUAAUGAAGGAGAAAACUCACCGAGGCAGUUCCAUAGGAUGGCAAG |
| | AUCCUGGUAUCGGUCUGCGAUUCCGACUCGUCCAACAUCAAUACAACCUAUUAAUUUCCCCUCGUC |
| | AAAAAUAAGGUUAUCAAGUGAGAAAUCACCAUGAGUGACGACUGAAUCCGGUGAGAAUGGCAAAAG |
| | CUUAUGCAUUUCUUUCCAGACUUGUUCAACAGGCCAGCCAUUACGCUCGUCAUCAAAAUCACUCGC |
| | AUCAACCAAACCGUUAUUCAUUCGUGAUUGCGCCUGAGCGAGACGAAAUACGCGAUCGCUGUUAAA |
| | AGGACAAUUACAAACAGGAAUCGAAUGCAACCGGCGCAGGAACACUGCCAGCGCAUCAACAAUAUU |
| | UUCACCUGAAUCAGGAUAUUCUUCUAAUACCUGGAAUGCUGUUUUCCCGGGGAUCGCAGUGGUGAG |
| | UAACCAUGCAUCAUCAGGAGUACGGAUAAAAUGCUUGAUGGUCGGAAGAGGCAUAAAUUCCGUCAG |
| | CCAGUUUAGUCUGACCAUCUCAUCUGUAACAUCAUUGGCAACGCUACCUUUGCCAUGUUUCAGAAA |
| | CAACUCUGGCGCAUCGGGCUUCCCAUACAAUCGAUAGAUUGUCGCACCUGAUUGCCCGACAUUAUC |
| | GCGAGCCCAUUUAUACCCAUAUAAAUCAGCAUCCAUGUUGGAAUUUAAUCGCGGCCUCGAGCAAGA |
| | CGUUUCCCGUUGAAUAUGGCUCAUAACACCCCUUGUAUUACUGUUUAUGUAAGCAGACAGUUUUAU |
| | UGUUCAUGAGCGGAUACAUAUUUGAAUGUAUUUAGAAAAAUAAACAAAUAGGGGUUCCGCGCACAU |
| | UUCCCCGAAAAGUGCCACCUAAAUUGUAAGCGUUAAUAUUUUGUUAAAAUUCGCGUUAAAUUUUUG |
| | UUAAAUCAGCUCAUUUUUUAACCAAUAGGCCGAAAUCGGCAAAAUCCCUUAUAAAUCAAAAGAAUA |
| | GACCGAGAUAGGGUUGAGUGGCCGCUACAGGGCGCUCCCAUUCGCCAUUCAGGCUGCGCAACUGUU |
| | GGGAAGGGCGUUUCGGUGCGGGCCUCUUCGCUAUUACGCCAGCUGGCGAAAGGGGGAUGUGCUGCA |
| | AGGCGAUUAAGUUGGGUAACGCCAGGGUUUUCCCAGUCACACGCGUAAUACGACUCACUAUAG |
| 7 | ATAGGCGGCGCATGAGAGAAGCCCAGACCAATTACCTACCCAAAATGGAGAAAGTTCACGTTGACA |
| | TCGAGGAAGACAGCCCATTCCTCAGAGCTTTGCAGCGGAGCTTCCCGCAGTTTGAGGTAGAAGCCA |
| | AGCAGGTCACTGATAATGACCATGCTAATGCCAGAGCGTTTTCGCATCTGGCTTCAAAACTGATCG |
| | AAACGGAGGTGGACCCATCCGACACGATCCTTGACATTGGAAGTGCGCCCGCCCGCAGAATGTATT |
| | CTAAGCACAAGTATCATTGTATCTGTCCGATGAGATGTGCGGAAGATCCGGACAGATTGTATAAGT |
| | ATGCAACTAAGCTGAAGAAAAACTGTAAGGAAATAACTGATAAGGAATTGGACAAGAAAATGAAGG |
| | AGCTCGCCGCCGTCATGAGCGACCCTGACCTGGAAACTGAGACTATGTGCCTCCACGACGACGAGT |
| | CGTGTCGCTACGAAGGGCAAGTCGCTGTTTACCAGGATGTATACGCGGTTGACGGACCGACAAGTC |
| | TCTATCACCAAGCCAATAAGGGAGTTAGAGTCGCCTACTGGATAGGCTTTGACACCACCCCTTTTA |
| | TGTTTAAGAACTTGGCTGGAGCATATCCATCATACTCTACCAACTGGGCCGACGAAACCGTGTTAA |
| | CGGCTCGTAACATAGGCCTATGCAGCTCTGACGTTATGGAGCGGTCACGTAGAGGGATGTCCATTC |
| | TTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATT |
| | ACACATGTCGGTGTGAGACTATAGTTAGTTGCGACGGGTACGTCGTTAAAAGAATAGCTATCAGTC |
| | CAGGCCTGTATGGGAAGCCTTCAGGCTATGCTGCTACGATGCACCGCGAGGGATTCTTGTGCTGCA |
| | AAGTGACAGACACATTGAACGGGGAGAGGGTCTCTTTTCCCGTGTGCACGTATGTGCCAGCTACAT |
| | TGTGTGACCAAATGACTGGCATACTGGCAACAGATGTCAGTGCGGACGACGCGCAAAAACTGCTGG |
| | TTGGGCTCAACCAGCGTATAGTCGTCAACGGTCGCACCCAGAGAAACACCAATACCATGAAAAATT |
| | ACCTTTTGCCCGTAGTGGCCCAGGCATTTGCTAGGTGGGCAAAGGAATATAAGGAAGATCAAGAAG |
| | ATGAAAGGCCACTAGGACTACGAGATAGACAGTTAGTCATGGGGTGTTGTTGGGCTTTTAGAAGGC |
| | ACAAGATAACATCTATTTATAAGCGCCCGGATACCCAAACCATCATCAAAGTGAACAGCGATTTCC |
| | ACTCATTCGTGCTGCCCAGGATAGGCAGTAACACATTGGAGATCGGGCTGAGAACAAGAATCAGGA |
| | AAATGTTAGAGGAGCACAAGGAGCCGTCACCTCTCATTACCGCCGAGGACGTACAAGAAGCTAAGT |
| | GCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCG |
| | GCTCAGTGGAGACACCTCGTGGCTTGATAAAGGTTACCAGCTACGATGGCGAGGACAAGATCGGCT |
| | CTTACGCTGTGCTTTCTCCGCAGGCTGTACTCAAGAGTGAAAAATTATCTTGCATCCACCCTCTCG |
| | CTGAACAAGTCATAGTGATAACACACTCTGGCCGAAAAGGGCGTTATGCCGTGGAACCATACCATG |
| | GTAAAGTAGTGGTGCCAGAGGGACATGCAATACCCGTCCAGGACTTTCAAGCTCTGAGTGAAAGTG |
| | CCACCATTGTGTACAACGAACGTGAGTTCGTAAACAGGTACCTGCACCATATTGCCACACATGGAG |
| | GAGCGCTGAACACTGATGAAGAATATTACAAAACTGTCAAGCCCAGCGAGCACGACGGCGAATACC |
| | TGTACGACATCGACAGGAAACAGTGCGTCAAGAAAGAACTAGTCACTGGGCTAGGGCTCACAGGCG |
| | AGCTGGTGGATCCTCCCTTCCATGAATTCGCCTACGAGAGTCTGAGAACACGACCAGCCGCTCCTT |
| | ACCAAGTACCAACCATAGGGGTGTATGGCGTGCCAGGATCAGGCAAGTCTGGCATCATTAAAAGCG |
| | CAGTCACCAAAAAAGATCTAGTGGTGAGCGCCAAGAAAGAAAACTGTGCAGAAATTATAAGGGACG |
| | TCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGC |
| | TCATAGCCATTATAAGACCTAAAAAGGCAGTGCTCTGCGGGGATCCCAAACAGTGCGGTTTTTTTA |
| | ACATGATGTGCCTGAAAGTGCATTTTAACCACGAGATTTGCACACAAGTCTTCCACAAAAGCATCT |
| | CTCGCCGTTGCACTAAATCTGTGACTTCGGTCGTCTCAACCTTGTTTTACGACAAAAAAATGAGAA |
| | CGACGAATCCGAAAGAGACTAAGATTGTGATTGACACTACCGGCAGTACCAAACCTAAGCAGGACG |
| | ATCTCATTCTCACTTGTTTCAGAGGGTGGGTGAAGCAGTTGCAAATAGATTACAAAGGCAACGAAA |
| | TAATGACGGCAGCTGCCTCTCAAGGGCTGACCCGTAAAGGTGTGTATGCCGTTCGGTACAAGGTGA |
| | ATGAAAATCCTCTGTACGCACCCACCTCAGAACATGTGAACGTCCTACTGACCCGCACGGAGGACC |
| | GCATCGTGTGGAAAACACTAGCCGGCGACCCATGGATAAAAACACTGACTGCCAAGTACCCTGGGA |
| | ATTTCACTGCCACGATAGAGGAGTGGCAAGCAGAGCATGATGCCATCATGAGGCACATCTTGGAGA |
| | GACCGGACCCTACCGACGTCTTCCAGAATAAGGCAAACGTGTGTTGGGCCAAGGCTTTAGTGCCGG |
| | TGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGG |
| | ACTCCGGTCTATTTTCTGCACCCACTGTTCCGTTATCCATTAGGAATAATCACTGGGATAACTCCC |
| | CGTCGCCTAACATGTACGGGCTGAATAAAGAAGTGGTCCGTCAGCTCTCTCGCAGGTACCCACAAC |
| | TGCCTCGGGCAGTTGCCACTGGAAGAGTCTATGACATGAACACTGGTACACTGCGCAATTATGATC |
| | CGCGCATAAACCTAGTACCTGTAAACAGAAGACTGCCTCATGCTTTAGTCCTCCACCATAATGAAC |
| | ACCCACAGAGTGACTTTTCTTCATTCGTCAGCAAATTGAAGGGCAGAACTGTCCTGGTGGTCGGGG |
| | AAAAGTTGTCCGTCCCAGGCAAAATGGTTGACTGGTTGTCAGACCGGCCTGAGGCTACCTTCAGAG |
| | CTCGGCTGGATTTAGGCATCCCAGGTGATGTGCCCAAATATGACATAATATTTGTTAATGTGAGGA |
| | CCCCATATAAATACCATCACTATCAGCAGTGTGAAGACCATGCCATTAAGCTTAGCATGTTGACCA |
| | AGAAAGCTTGTCTGCATCTGAATCCCGGCGGAACCTGTGTCAGCATAGGTTATGGTTACGCTGACA |
| | GGGCCAGCGAAAGCATCATTGGTGCTATAGCGCGGCAGTTCAAGTTTTCCCGGGTATGCAAACCGA |
| | AATCCTCACTTGAAGAGACGGAAGTTCTGTTTGTATTCATTGGGTACGATCGCAAGGCCCGTACGC |
| | ACAATCCTTACAAGCTTTCATCAACCTTGACCAACATTTATACAGGTTCCAGACTCCACGAAGCCG |
| | GATGTGCACCCTCATATCATGTGGTGCGAGGGGATATTGCCACGGCCACCGAAGGAGTGATTATAA |
| | ATGCTGCTAACAGCAAAGGACAACCTGGCGGAGGGGTGTGCGGAGCGCTGTATAAGAAATTCCCGG |
| | AAAGCTTCGATTTACAGCCGATCGAAGTAGGAAAAGCGCGACTGGTCAAAGGTGCAGCTAAACATA |
| | TCATTCATGCCGTAGGACCAAACTTCAACAAAGTTTCGGAGGTTGAAGGTGACAAACAGTTGGCAG |
| | AGGCTTATGAGTCCATCGCTAAGATTGTCAACGATAACAATTACAAGTCAGTAGCGATTCCACTGT |
| | TGTCCACCGGCATCTTTTCCGGGAACAAAGATCGACTAACCCAATCATTGAACCATTTGCTGACAG |
| | CTTTAGACACCACTGATGCAGATGTAGCCATATACTGCAGGGACAAGAAATGGGAAATGACTCTCA |
| | AGGAAGCAGTGGCTAGGAGAGAAGCAGTGGAGGAGATATGCATATCCGACGACTCTTCAGTGACAG |
| | AACCTGATGCAGAGCTGGTGAGGGTGCATCCGAAGAGTTCTTTGGCTGGAAGGAAGGGCTACAGCA |
| | CAAGCGATGGCAAAACTTTCTCATATTTGGAAGGGACCAAGTTTCACCAGGCGGCCAAGGATATAG |
| | CAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGATTCGGAAGCCTCCACACCACCTA |
| | GCACGCTGCCTTGCTTGTGCATCCATGCCATGACTCCAGAAAGAGTACAGCGCCTAAAAGCCTCAC |
| | GTCCAGAACAAATTACTGTGTGCTCATCCTTTCCATTGCCGAAGTATAGAATCACTGGTATGCAGA |
| | AGATCCAATGCTCCCAGCCTATATTGTTCTCACCGAAAGTGCCTGCGTATATTCATCCAAGGAAGT |
| | ATCTCGTGGAAACACCACCGGTAGACGAGACTCCGGAGCCATCGGCAGAGAACCAATCCACAGAGG |
| | GGACACCTGAACAACCACCACTTATAACCGAGGATGAGACCAGGACTAGAACGCCTGAGCCGATCA |
| | TCATCGAAGAGGAAGAAGAGGATAGCATAAGTTTGCTGTCAGATGGCCCGACCCACCAGGTGCTGC |
| | AAGTCGAGGCAGACATTCACGGGCCGCCCTCTGTATCTAGCTCATCCTGGTCCATTCCTCATGCAT |
| | CCGACTTTGATGTGGACAGTTTATCCATACTTGACACCCTGGAGGGAGCTAGCGTGACCAGCGGGG |
| | CAACGTCAGCCGAGACTAACTCTTACTTCGCAAAGAGTATGGAGTTTCTGGCGCGACCGGTGCCTG |
| | CGCCTCGAACAGTATTCAGGAACCCTCCACATCCCGCTCCGCGCACAAGAACACCGTCACTTGCAC |
| | CCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGG |
| | TCTCCAACCCGCCAGGCGTAAATAGGGTGATTACAAGAGAGGAGTTTGAGGCGTTCGTAGCACAAC |
| | AACAATGACGGTTTGATGCGGGTGCATACATCTTTTCCTCCGACACCGGTCAAGGGCATTTACAAC |
| | AAAAATCAGTAAGGCAAACGGTGCTATCCGAAGTGGTGTTGGAGAGGACCGAATTGGAGATTTCGT |
| | ATGCCCCGCGCCTCGACCAAGAAAAAGAAGAATTACTACGCAAGAAATTACAGTTAAATCCCACAC |
| | CTGCTAACAGAAGCAGATACCAGTCCAGGAAGGTGGAGAACATGAAAGCCATAACAGCTAGACGTA |
| | TTCTGCAAGGCCTAGGGCATTATTTGAAGGCAGAAGGAAAAGTGGAGTGCTACCGAACCCTGCATC |
| | CTGTTCCTTTGTATTCATCTAGTGTGAACCGTGCCTTTTCAAGCCCCAAGGTCGCAGTGGAAGCCT |
| | GTAACGCCATGTTGAAAGAGAACTTTCCGACTGTGGCTTCTTACTGTATTATTCCAGAGTACGATG |
| | CCTATTTGGACATGGTTGACGGAGCTTCATGCTGCTTAGACACTGCCAGTTTTTGCCCTGCAAAGC |
| | TGCGCAGCTTTCCAAAGAAACACTCCTATTTGGAACCCACAATACGATCGGCAGTGCCTTCAGCGA |
| | TCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATA |
| | ATGAATATTGGGAAACGTTTAAAGAAAACCCCATCAGGCTTACTGAAGAAAACGTGGTAAATTACA |
| | TTACCAAATTAAAAGGACCAAAAGCTGCTGCTCTTTTTGCGAAGACACATAATTTGAATATGTTGC |
| | AGGACATACCAATGGACAGGTTTGTAATGGACTTAAAGAGAGACGTGAAAGTGACTCCAGGAACAA |
| | AACATACTGAAGAACGGCCCAAGGTACAGGTGATCCAGGCTGCCGATCCGCTAGCAACAGCGTATC |
| | TGTGCGGAATCCACCGAGAGCTGGTTAGGAGATTAAATGCGGTCCTGCTTCCGAACATTCATACAC |
| | TGTTTGATATGTCGGCTGAAGACTTTGACGCTATTATAGCCGAGCACTTCCAGCCTGGGGATTGTG |
| | TTCTGGAAACTGACATCGCGTCGTTTGATAAAAGTGAGGACGACGCCATGGCTCTGACCGCGTTAA |
| | TGATTCTGGAAGACTTAGGTGTGGACGCAGAGCTGTTGACGCTGATTGAGGCGGCTTTCGGCGAAA |
| | TTTCATCAATACATTTGCCCACTAAAACTAAATTTAAATTCGGAGCCATGATGAAATCTGGAATGT |
| | TCCTCACACTGTTTGTGAACACAGTCATTAACATTGTAATCGCAAGCAGAGTGTTGAGAGAACGGC |
| | TAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGG |
| | GCGAGAAAGCGCCTTATTTCTGTGGAGGGTTTATTTTGTGTGACTCCGTGACCGGCACAGCGTGCC |
| | GTGTGGCAGACCCCCTAAAAAGGCTGTTTAAGCTTGGCAAACCTCTGGCAGCAGACGATGAACATG |
| | ATGATGACAGGAGAAGGGCATTGCATGAAGAGTCAACACGCTGGAACCGAGTGGGTATTCTTTCAG |
| | AGCTGTGCAAGGCAGTAGAATCAAGGTATGAAACCGTAGGAACTTCCATCATAGTTATGGCCATGA |
| | CTACTCTAGCTAGCAGTGTTAAATCATTCAGCTACCTGAGAGGGGCCCCTATAACTCTCTACGGCT |
| | AACCTGAATGGACTACGACATAGTCTAGTCCGCCAAG |
| 8 | AUAGGCGGCGCAUGAGAGAAGCCCAGACCAAUUACCUACCCAAAAUGGAGAAAGUUCACGUUGACA |
| | UCGAGGAAGACAGCCCAUUCCUCAGAGCUUUGCAGCGGAGCUUCCCGCAGUUUGAGGUAGAAGCCA |
| | AGCAGGUCACUGAUAAUGACCAUGCUAAUGCCAGAGCGUUUUCGCAUCUGGCUUCAAAACUGAUCG |
| | AAACGGAGGUGGACCCAUCCGACACGAUCCUUGACAUUGGAAGUGCGCCCGCCCGCAGAAUGUAUU |
| | CUAAGCACAAGUAUCAUUGUAUCUGUCCGAUGAGAUGUGCGGAAGAUCCGGACAGAUUGUAUAAGU |
| | AUGCAACUAAGCUGAAGAAAAACUGUAAGGAAAUAACUGAUAAGGAAUUGGACAAGAAAAUGAAGG |
| | AGCUCGCCGCCGUCAUGAGCGACCCUGACCUGGAAACUGAGACUAUGUGCCUCCACGACGACGAGU |
| | CGUGUCGCUACGAAGGGCAAGUCGCUGUUUACCAGGAUGUAUACGCGGUUGACGGACCGACAAGUC |
| | UCUAUCACCAAGCCAAUAAGGGAGUUAGAGUCGCCUACUGGAUAGGCUUUGACACCACCCCUUUUA |
| | UGUUUAAGAACUUGGCUGGAGCAUAUCCAUCAUACUCUACCAACUGGGCCGACGAAACCGUGUUAA |
| | CGGCUCGUAACAUAGGCCUAUGCAGCUCUGACGUUAUGGAGCGGUCACGUAGAGGGAUGUCCAUUC |
| | UUAGAAAGAAGUAUUUGAAACCAUCCAACAAUGUUCUAUUCUCUGUUGGCUCGACCAUCUACCACG |
| | AGAAGAGGGACUUACUGAGGAGCUGGCACCUGCCGUCUGUAUUUCACUUACGUGGCAAGCAAAAUU |
| | ACACAUGUCGGUGUGAGACUAUAGUUAGUUGCGACGGGUACGUCGUUAAAAGAAUAGCUAUCAGUC |
| | CAGGCCUGUAUGGGAAGCCUUCAGGCUAUGCUGCUACGAUGCACCGCGAGGGAUUCUUGUGCUGCA |
| | AAGUGACAGACACAUUGAACGGGGAGAGGGUCUCUUUUCCCGUGUGCACGUAUGUGCCAGCUACAU |
| | UGUGUGACCAAAUGACUGGCAUACUGGCAACAGAUGUCAGUGCGGACGACGCGCAAAAACUGCUGG |
| | UUGGGCUCAACCAGCGUAUAGUCGUCAACGGUCGCACCCAGAGAAACACCAAUACCAUGAAAAAUU |
| | ACCUUUUGCCCGUAGUGGCCCAGGCAUUUGCUAGGUGGGCAAAGGAAUAUAAGGAAGAUCAAGAAG |
| | AUGAAAGGCCACUAGGACUACGAGAUAGACAGUUAGUCAUGGGGUGUUGUUGGGCUUUUAGAAGGC |
| | ACAAGAUAACAUCUAUUUAUAAGCGCCCGGAUACCCAAACCAUCAUCAAAGUGAACAGCGAUUUCC |
| | ACUCAUUCGUGCUGCCCAGGAUAGGCAGUAACACAUUGGAGAUCGGGCUGAGAACAAGAAUCAGGA |
| | AAAUGUUAGAGGAGCACAAGGAGCCGUCACCUCUCAUUACCGCCGAGGACGUACAAGAAGCUAAGU |
| | GCGCAGCCGAUGAGGCUAAGGAGGUGCGUGAAGCCGAGGAGUUGCGCGCAGCUCUACCACCUUUGG |
| | CAGCUGAUGUUGAGGAGCCCACUCUGGAAGCCGAUGUCGACUUGAUGUUACAAGAGGCUGGGGCCG |
| | GCUCAGUGGAGACACCUCGUGGCUUGAUAAAGGUUACCAGCUACGAUGGCGAGGACAAGAUCGGCU |
| | CUUACGCUGUGCUUUCUCCGCAGGCUGUACUCAAGAGUGAAAAAUUAUCUUGCAUCCACCCUCUCG |
| | CUGAACAAGUCAUAGUGAUAACACACUCUGGCCGAAAAGGGCGUUAUGCCGUGGAACCAUACCAUG |
| | GUAAAGUAGUGGUGCCAGAGGGACAUGCAAUACCCGUCCAGGACUUUCAAGCUCUGAGUGAAAGUG |
| | CCACCAUUGUGUACAACGAACGUGAGUUCGUAAACAGGUACCUGCACCAUAUUGCCACACAUGGAG |
| | GAGCGCUGAACACUGAUGAAGAAUAUUACAAAACUGUCAAGCCCAGCGAGCACGACGGCGAAUACC |
| | UGUACGACAUCGACAGGAAACAGUGCGUCAAGAAAGAACUAGUCACUGGGCUAGGGCUCACAGGCG |
| | AGCUGGUGGAUCCUCCCUUCCAUGAAUUCGCCUACGAGAGUCUGAGAACACGACCAGCCGCUCCUU |
| | ACCAAGUACCAACCAUAGGGGUGUAUGGCGUGCCAGGAUCAGGCAAGUCUGGCAUCAUUAAAAGCG |
| | CAGUCACCAAAAAAGAUCUAGUGGUGAGCGCCAAGAAAGAAAACUGUGCAGAAAUUAUAAGGGACG |
| | UCAAGAAAAUGAAAGGGCUGGACGUCAAUGCCAGAACUGUGGACUCAGUGCUCUUGAAUGGAUGCA |
| | AACACCCCGUAGAGACCCUGUAUAUUGACGAAGCUUUUGCUUGUCAUGCAGGUACUCUCAGAGCGC |
| | UCAUAGCCAUUAUAAGACCUAAAAAGGCAGUGCUCUGCGGGGAUCCCAAACAGUGCGGUUUUUUUA |
| | ACAUGAUGUGCCUGAAAGUGCAUUUUAACCACGAGAUUUGCACACAAGUCUUCCACAAAAGCAUCU |
| | CUCGCCGUUGCACUAAAUCUGUGACUUCGGUCGUCUCAACCUUGUUUUACGACAAAAAAAUGAGAA |
| | CGACGAAUCCGAAAGAGACUAAGAUUGUGAUUGACACUACCGGCAGUACCAAACCUAAGCAGGACG |
| | AUCUCAUUCUCACUUGUUUCAGAGGGUGGGUGAAGCAGUUGCAAAUAGAUUACAAAGGCAACGAAA |
| | UAAUGACGGCAGCUGCCUCUCAAGGGCUGACCCGUAAAGGUGUGUAUGCCGUUCGGUACAAGGUGA |
| | AUGAAAAUCCUCUGUACGCACCCACCUCAGAACAUGUGAACGUCCUACUGACCCGCACGGAGGACC |
| | GCAUCGUGUGGAAAACACUAGCCGGCGACCCAUGGAUAAAAACACUGACUGCCAAGUACCCUGGGA |
| | AUUUCACUGCCACGAUAGAGGAGUGGCAAGCAGAGCAUGAUGCCAUCAUGAGGCACAUCUUGGAGA |
| | GACCGGACCCUACCGACGUCUUCCAGAAUAAGGCAAACGUGUGUUGGGCCAAGGCUUUAGUGCCGG |
| | UGCUGAAGACCGCUGGCAUAGACAUGACCACUGAACAAUGGAACACUGUGGAUUAUUUUGAAACGG |
| | ACAAAGCUCACUCAGCAGAGAUAGUAUUGAACCAACUAUGCGUGAGGUUCUUUGGACUCGAUCUGG |
| | ACUCCGGUCUAUUUUCUGCACCCACUGUUCCGUUAUCCAUUAGGAAUAAUCACUGGGAUAACUCCC |
| | CGUCGCCUAACAUGUACGGGCUGAAUAAAGAAGUGGUCCGUCAGCUCUCUCGCAGGUACCCACAAC |
| | UGCCUCGGGCAGUUGCCACUGGAAGAGUCUAUGACAUGAACACUGGUACACUGCGCAAUUAUGAUC |
| | CGCGCAUAAACCUAGUACCUGUAAACAGAAGACUGCCUCAUGCUUUAGUCCUCCACCAUAAUGAAC |
| | ACCCACAGAGUGACUUUUCUUCAUUCGUCAGCAAAUUGAAGGGCAGAACUGUCCUGGUGGUCGGGG |
| | AAAAGUUGUCCGUCCCAGGCAAAAUGGUUGACUGGUUGUCAGACCGGCCUGAGGCUACCUUCAGAG |
| | CUCGGCUGGAUUUAGGCAUCCCAGGUGAUGUGCCCAAAUAUGACAUAAUAUUUGUUAAUGUGAGGA |
| | CCCCAUAUAAAUACCAUCACUAUCAGCAGUGUGAAGACCAUGCCAUUAAGCUUAGCAUGUUGACCA |
| | AGAAAGCUUGUCUGCAUCUGAAUCCCGGCGGAACCUGUGUCAGCAUAGGUUAUGGUUACGCUGACA |
| | GGGCCAGCGAAAGCAUCAUUGGUGCUAUAGCGCGGCAGUUCAAGUUUUCCCGGGUAUGCAAACCGA |
| | AAUCCUCACUUGAAGAGACGGAAGUUCUGUUUGUAUUCAUUGGGUACGAUCGCAAGGCCCGUACGC |
| | ACAAUCCUUACAAGCUUUCAUCAACCUUGACCAACAUUUAUACAGGUUCCAGACUCCACGAAGCCG |
| | GAUGUGCACCCUCAUAUCAUGUGGUGCGAGGGGAUAUUGCCACGGCCACCGAAGGAGUGAUUAUAA |
| | AUGCUGCUAACAGCAAAGGACAACCUGGCGGAGGGGUGUGCGGAGCGCUGUAUAAGAAAUUCCCGG |
| | AAAGCUUCGAUUUACAGCCGAUCGAAGUAGGAAAAGCGCGACUGGUCAAAGGUGCAGCUAAACAUA |
| | UCAUUCAUGCCGUAGGACCAAACUUCAACAAAGUUUCGGAGGUUGAAGGUGACAAACAGUUGGCAG |
| | AGGCUUAUGAGUCCAUCGCUAAGAUUGUCAACGAUAACAAUUACAAGUCAGUAGCGAUUCCACUGU |
| | UGUCCACCGGCAUCUUUUCCGGGAACAAAGAUCGACUAACCCAAUCAUUGAACCAUUUGCUGACAG |
| | CUUUAGACACCACUGAUGCAGAUGUAGCCAUAUACUGCAGGGACAAGAAAUGGGAAAUGACUCUCA |
| | AGGAAGCAGUGGCUAGGAGAGAAGCAGUGGAGGAGAUAUGCAUAUCCGACGACUCUUCAGUGACAG |
| | AACCUGAUGCAGAGCUGGUGAGGGUGCAUCCGAAGAGUUCUUUGGCUGGAAGGAAGGGCUACAGCA |
| | CAAGCGAUGGCAAAACUUUCUCAUAUUUGGAAGGGACCAAGUUUCACCAGGCGGCCAAGGAUAUAG |
| | CAGAAAUUAAUGCCAUGUGGCCCGUUGCAACGGAGGCCAAUGAGCAGGUAUGCAUGUAUAUCCUCG |
| | GAGAAAGCAUGAGCAGUAUUAGGUCGAAAUGCCCCGUCGAAGAUUCGGAAGCCUCCACACCACCUA |
| | GCACGCUGCCUUGCUUGUGCAUCCAUGCCAUGACUCCAGAAAGAGUACAGCGCCUAAAAGCCUCAC |
| | GUCCAGAACAAAUUACUGUGUGCUCAUCCUUUCCAUUGCCGAAGUAUAGAAUCACUGGUAUGCAGA |
| | AGAUCCAAUGCUCCCAGCCUAUAUUGUUCUCACCGAAAGUGCCUGCGUAUAUUCAUCCAAGGAAGU |
| | AUCUCGUGGAAACACCACCGGUAGACGAGACUCCGGAGCCAUCGGCAGAGAACCAAUCCACAGAGG |
| | GGACACCUGAACAACCACCACUUAUAACCGAGGAUGAGACCAGGACUAGAACGCCUGAGCCGAUCA |
| | UCAUCGAAGAGGAAGAAGAGGAUAGCAUAAGUUUGCUGUCAGAUGGCCCGACCCACCAGGUGCUGC |
| | AAGUCGAGGCAGACAUUCACGGGCCGCCCUCUGUAUCUAGCUCAUCCUGGUCCAUUCCUCAUGCAU |
| | CCGACUUUGAUGUGGACAGUUUAUCCAUACUUGACACCCUGGAGGGAGCUAGCGUGACCAGCGGGG |
| | CAACGUCAGCCGAGACUAACUCUUACUUCGCAAAGAGUAUGGAGUUUCUGGCGCGACCGGUGCCUG |
| | CGCCUCGAACAGUAUUCAGGAACCCUCCACAUCCCGCUCCGCGCACAAGAACACCGUCACUUGCAC |
| | CCAGCAGGGCCUGCUCGAGAACCAGCCUAGUUUCCACCCCGCCAGGCGUGAAUAGGGUGAUCACUA |
| | GAGAGGAGCUCGAGGCGCUUACCCCGUCACGCACUCCUAGCAGGUCGGUCUCGAGAACCAGCCUGG |
| | UCUCCAACCCGCCAGGCGUAAAUAGGGUGAUUACAAGAGAGGAGUUUGAGGCGUUCGUAGCACAAC |
| | AACAAUGACGGUUUGAUGCGGGUGCAUACAUCUUUUCCUCCGACACCGGUCAAGGGCAUUUACAAC |
| | AAAAAUCAGUAAGGCAAACGGUGCUAUCCGAAGUGGUGUUGGAGAGGACCGAAUUGGAGAUUUCGU |
| | AUGCCCCGCGCCUCGACCAAGAAAAAGAAGAAUUACUACGCAAGAAAUUACAGUUAAAUCCCACAC |
| | CUGCUAACAGAAGCAGAUACCAGUCCAGGAAGGUGGAGAACAUGAAAGCCAUAACAGCUAGACGUA |
| | UUCUGCAAGGCCUAGGGCAUUAUUUGAAGGCAGAAGGAAAAGUGGAGUGCUACCGAACCCUGCAUC |
| | CUGUUCCUUUGUAUUCAUCUAGUGUGAACCGUGCCUUUUCAAGCCCCAAGGUCGCAGUGGAAGCCU |
| | GUAACGCCAUGUUGAAAGAGAACUUUCCGACUGUGGCUUCUUACUGUAUUAUUCCAGAGUACGAUG |
| | CCUAUUUGGACAUGGUUGACGGAGCUUCAUGCUGCUUAGACACUGCCAGUUUUUGCCCUGCAAAGC |
| | UGCGCAGCUUUCCAAAGAAACACUCCUAUUUGGAACCCACAAUACGAUCGGCAGUGCCUUCAGCGA |
| | UCCAGAACACGCUCCAGAACGUCCUGGCAGCUGCCACAAAAAGAAAUUGCAAUGUCACGCAAAUGA |
| | GAGAAUUGCCCGUAUUGGAUUCGGCGGCCUUUAAUGUGGAAUGCUUCAAGAAAUAUGCGUGUAAUA |
| | AUGAAUAUUGGGAAACGUUUAAAGAAAACCCCAUCAGGCUUACUGAAGAAAACGUGGUAAAUUACA |
| | UUACCAAAUUAAAAGGACCAAAAGCUGCUGCUCUUUUUGCGAAGACACAUAAUUUGAAUAUGUUGC |
| | AGGACAUACCAAUGGACAGGUUUGUAAUGGACUUAAAGAGAGACGUGAAAGUGACUCCAGGAACAA |
| | AACAUACUGAAGAACGGCCCAAGGUACAGGUGAUCCAGGCUGCCGAUCCGCUAGCAACAGCGUAUC |
| | UGUGCGGAAUCCACCGAGAGCUGGUUAGGAGAUUAAAUGCGGUCCUGCUUCCGAACAUUCAUACAC |
| | UGUUUGAUAUGUCGGCUGAAGACUUUGACGCUAUUAUAGCCGAGCACUUCCAGCCUGGGGAUUGUG |
| | UUCUGGAAACUGACAUCGCGUCGUUUGAUAAAAGUGAGGACGACGCCAUGGCUCUGACCGCGUUAA |
| | UGAUUCUGGAAGACUUAGGUGUGGACGCAGAGCUGUUGACGCUGAUUGAGGCGGCUUUCGGCGAAA |
| | UUUCAUCAAUACAUUUGCCCACUAAAACUAAAUUUAAAUUCGGAGCCAUGAUGAAAUCUGGAAUGU |
| | UCCUCACACUGUUUGUGAACACAGUCAUUAACAUUGUAAUCGCAAGCAGAGUGUUGAGAGAACGGC |
| | UAACCGGAUCACCAUGUGCAGCAUUCAUUGGAGAUGACAAUAUCGUGAAAGGAGUCAAAUCGGACA |
| | AAUUAAUGGCAGACAGGUGCGCCACCUGGUUGAAUAUGGAAGUCAAGAUUAUAGAUGCUGUGGUGG |
| | GCGAGAAAGCGCCUUAUUUCUGUGGAGGGUUUAUUUUGUGUGACUCCGUGACCGGCACAGCGUGCC |
| | GUGUGGCAGACCCCCUAAAAAGGCUGUUUAAGCUUGGCAAACCUCUGGCAGCAGACGAUGAACAUG |
| | AUGAUGACAGGAGAAGGGCAUUGCAUGAAGAGUCAACACGCUGGAACCGAGUGGGUAUUCUUUCAG |
| | AGCUGUGCAAGGCAGUAGAAUCAAGGUAUGAAACCGUAGGAACUUCCAUCAUAGUUAUGGCCAUGA |
| | CUACUCUAGCUAGCAGUGUUAAAUCAUUCAGCUACCUGAGAGGGGCCCCUAUAACUCUCUACGGCU |
| | AACCUGAAUGGACUACGACAUAGUCUAGUCCGCCAAG |
| 9 | ATGGTGAGCAAGGGCGAGGAGCTGTTCACCGGGGTGGTGCCCATCCTGGTCGAGCTGGACGGCGAC |
| | GTAAACGGCCACAAGTTCAGCGTGTCCGGCGAGGGCGAGGGCGATGCCACCTACGGCAAGCTGACC |
| | CTGAAGTTCATCTGCACCACCGGCAAGCTGCCCGTGCCCTGGCCCACCCTCGTGACCACCCTGACC |
| | TACGGCGTGCAGTGCTTCAGCCGCTACCCCGACCACATGAAGCAGCACGACTTCTTCAAGTCCGCC |
| | ATGCCCGAAGGCTACGTCCAGGAGCGCACCATCTTCTTCAAGGACGACGGCAACTACAAGACCCGC |
| | GCCGAGGTGAAGTTCGAGGGCGACACCCTGGTGAACCGCATCGAGCTGAAGGGCATCGACTTCAAG |
| | GAGGACGGCAACATCCTGGGGCACAAGCTGGAGTACAACTACAACAGCCACAACGTCTATATCATG |
| | GCCGACAAGCAGAAGAACGGCATCAAGGTGAACTTCAAGATCCGCCACAACATCGAGGACGGCAGC |
| | GTGCAGCTCGCCGACCACTACCAGCAGAACACCCCCATCGGCGACGGCCCCGTGCTGCTGCCCGAC |
| | AACCACTACCTGAGCACCCAGTCCGCCCTGAGCAAAGACCCCAACGAGAAGCGCGATCACATGGTC |
| | CTGCTGGAGTTCGTGACCGCCGCCGGGATCACTCTCGGCATGGACGAGCTGTACAAGTGATAA |
| 10 | AUGGUGAGCAAGGGCGAGGAGCUGUUCACCGGGGUGGUGCCCAUCCUGGUCGAGCUGGACGGCGAC |
| | GUAAACGGCCACAAGUUCAGCGUGUCCGGCGAGGGCGAGGGCGAUGCCACCUACGGCAAGCUGACC |
| | CUGAAGUUCAUCUGCACCACCGGCAAGCUGCCCGUGCCCUGGCCCACCCUCGUGACCACCCUGACC |
| | UACGGCGUGCAGUGCUUCAGCCGCUACCCCGACCACAUGAAGCAGCACGACUUCUUCAAGUCCGCC |
| | AUGCCCGAAGGCUACGUCCAGGAGCGCACCAUCUUCUUCAAGGACGACGGCAACUACAAGACCCGC |
| | GCCGAGGUGAAGUUCGAGGGCGACACCCUGGUGAACCGCAUCGAGCUGAAGGGCAUCGACUUCAAG |
| | GAGGACGGCAACAUCCUGGGGCACAAGCUGGAGUACAACUACAACAGCCACAACGUCUAUAUCAUG |
| | GCCGACAAGCAGAAGAACGGCAUCAAGGUGAACUUCAAGAUCCGCCACAACAUCGAGGACGGCAGC |
| | GUGCAGCUCGCCGACCACUACCAGCAGAACACCCCCAUCGGCGACGGCCCCGUGCUGCUGCCCGAC |
| | AACCACUACCUGAGCACCCAGUCCGCCCUGAGCAAAGACCCCAACGAGAAGCGCGAUCACAUGGUC |
| | CUGCUGGAGUUCGUGACCGCCGCCGGGAUCACUCUCGGCAUGGACGAGCUGUACAAGUGAUAA |
| 11 | MVSKGEELFTGVVPILVELDGDVNGHKFSVSGEGEGDATYGKLTLKFICTTGKLPVPWPTLVTTLT |
| | YGVQCFSRYPDHMKQHDFFKSAMPEGYVQERTIFFKDDGNYKTRAEVKFEGDTLVNRIELKGIDFK |
| | EDGNILGHKLEYNYNSHNVYIMADKQKNGIKVNFKIRHNIEDGSVQLADHYQQNTPIGDGPVLLPD |
| | NHYLSTQSALSKDPNEKRDHMVLLEFVTAAGITLGMDELYK** |
| 12 | see 9 |
| 13 | see 10 |
| 14 | see 11 |
| 15 | ATGGACCCTAATACGGTCTCTTCTTTCCAAGTGGATTGCTTTCTGTGGCATGTTAGAAAGAGAGTG |
| | GCTGATCAAGAATTGGGTGATGCACCGTTCTTGGATAGACTGCGACGCGATCAGAAGTCCCTTCGC |
| | GGTAGAGGCAGCACGCTTGGGTTGGACATCAAAACTGCGACTCGAGCCGGGAAACAAATTGTAGAG |
| | AGGATTCTTAAGGAGGAAAGTGACGAGGCACTCAAAATGACAATGGCAAGCGTACCGGCCAGTCGG |
| | TATTTGACAGACATGACTCTCGAAGAGATGTCACGAGATTGGTCCATGCTCATACCGAAGCAGAAG |
| | GTGGCCGGTCCTCTCTGTATTAGAATGGATCAGGCGATTATGGATAAGAATATCATTTTGAAGGCC |
| | AATTTTTCTGTCATTTTCGACAGACTGGAAACATTGATTCTGCTCCGGGCTTTCACCGAGGAAGGT |
| | GCAATCGTAGGTGAGATCAGTCCGCTCCCTAGCCTTCCCGGTCATACCGCAGAGGATGTTAAAAAC |
| | GCTGTGGGTGTTTTGATTGGGGGCTTGGAGTGGAACGACAACACAGTCAGGGTGAGCGAGACACTT |
| | CAACGGTTTGCATGGCGGAGTAGTAACGAGAACGGACGCCCGCCCCTCACACCAAAGCAGAAACGA |
| | GAGATGGCAGGAACTATCCGATCTGAGGTCTGATAA |
| 16 | AUGGACCCUAAUACGGUCUCUUCUUUCCAAGUGGAUUGCUUUCUGUGGCAUGUUAGAAAGAGAGUG |
| | GCUGAUCAAGAAUUGGGUGAUGCACCGUUCUUGGAUAGACUGCGACGCGAUCAGAAGUCCCUUCGC |
| | GGUAGAGGCAGCACGCUUGGGUUGGACAUCAAAACUGCGACUCGAGCCGGGAAACAAAUUGUAGAG |
| | AGGAUUCUUAAGGAGGAAAGUGACGAGGCACUCAAAAUGACAAUGGCAAGCGUACCGGCCAGUCGG |
| | UAUUUGACAGACAUGACUCUCGAAGAGAUGUCACGAGAUUGGUCCAUGCUCAUACCGAAGCAGAAG |
| | GUGGCCGGUCCUCUCUGUAUUAGAAUGGAUCAGGCGAUUAUGGAUAAGAAUAUCAUUUUGAAGGCC |
| | AAUUUUUCUGUCAUUUUCGACAGACUGGAAACAUUGAUUCUGCUCCGGGCUUUCACCGAGGAAGGU |
| | GCAAUCGUAGGUGAGAUCAGUCCGCUCCCUAGCCUUCCCGGUCAUACCGCAGAGGAUGUUAAAAAC |
| | GCUGUGGGUGUUUUGAUUGGGGGCUUGGAGUGGAACGACAACACAGUCAGGGUGAGCGAGACACUU |
| | CAACGGUUUGCAUGGCGGAGUAGUAACGAGAACGGACGCCCGCCCCUCACACCAAAGCAGAAACGA |
| | GAGAUGGCAGGAACUAUCCGAUCUGAGGUCUGAUAA |
| 17 | MDPNTVSSFQVDCFLWHVRKRVADQELGDAPFLDRLRRDQKSLRGRGSTLGLDIKTATRAGKQIVE |
| | RILKEESDEALKMTMASVPASRYLTDMTLEEMSRDWSMLIPKQKVAGPLCIRMDQAIMDKNIILKA |
| | NFSVIFDRLETLILLRAFTEEGAIVGEISPLPSLPGHTAEDVKNAVGVLIGGLEWNDNTVRVSETL |
| | QRFAWRSSNENGRPPLTPKQKREMAGTIRSEV** |
| 18 | ATGGGACAAGAGCAGGACACACCGTGGATTCTGTCTACCGGACATATAAGTACGCAAAAGCGCGAG |
| | GATGGGCAGCAGACTCCCCGGCTCGAGCACCACAATAGTACACGGCTTATGGACCATTGCCAGAAG |
| | ACAATGAACCAGGTAGTAATGCCAAAGCAAATCGTCTATTGGAAACAATGGCTCTCTCTGAGGAGT |
| | CCCACACCTGTGTCCCTGAAGACCCGCGTCTTGAAAAGGTGGCGCTTGTTCTCTAAACATGAGTGG |
| | ACATCTTGATAA |
| 19 | AUGGGACAAGAGCAGGACACACCGUGGAUUCUGUCUACCGGACAUAUAAGUACGCAAAAGCGCGAG |
| | GAUGGGCAGCAGACUCCCCGGCUCGAGCACCACAAUAGUACACGGCUUAUGGACCAUUGCCAGAAG |
| | ACAAUGAACCAGGUAGUAAUGCCAAAGCAAAUCGUCUAUUGGAAACAAUGGCUCUCUCUGAGGAGU |
| | CCCACACCUGUGUCCCUGAAGACCCGCGUCUUGAAAAGGUGGCGCUUGUUCUCUAAACAUGAGUGG |
| | ACAUCUUGAUAA |
| 20 | MGQEQDTPWILSTGHISTQKREDGQQTPRLEHHNSTRLMDHCQKTMNQVVMPKQIVYWKQWLSLRS |
| | PTPVSLKTRVLKRWRLFSKHEWTS** |
| 21 | ATGAGTAAAATATATATTGACGAACGGAGTAACGCTGAGATCGTTTGTGAGGCTATTAAGACAATA |
| | GGGATCGAAGGAGCGACGGCGGCGCAACTCACCCGCCAGTTGAATATGGAAAAACGAGAAGTCAAT |
| | AAGGCGCTGTATGACCTCCAGCGATCAGCCATGGTTTACTCTAGTGATGATATTCCACCAAGATGG |
| | TTCATGACGACCGAGGCTGATAAACCTGATGCAGATGCAATGGCCGACGTCATTATTGACGATGTG |
| | AGCCGCGAGAAGTCCATGCGCGAGGATCATAAATCTTTTGATGATGTCATCCCGGCCAAAAAAATA |
| | ATCGACTGGAAAGGAGCGAATCCCGTGACCGTCATAAACGAGTATTGCCAGATAACACGACGAGAT |
| | TGGAGCTTCAGGATTGAGTCCGTTGGTCCGTCTAACTCTCCCACCTTCTATGCGTGTGTTGACATA |
| | GACGGTAGGGTCTTTGACAAAGCTGATGGTAAATCCAAGCGAGACGCCAAGAATAATGCAGCCAAA |
| | CTCGCCGTAGATAAGTTGCTGGGATACGTGATTATCCGATTCTAATAA |
| 22 | AUGAGUAAAAUAUAUAUUGACGAACGGAGUAACGCUGAGAUCGUUUGUGAGGCUAUUAAGACAAUA |
| | GGGAUCGAAGGAGCGACGGCGGCGCAACUCACCCGCCAGUUGAAUAUGGAAAAACGAGAAGUCAAU |
| | AAGGCGCUGUAUGACCUCCAGCGAUCAGCCAUGGUUUACUCUAGUGAUGAUAUUCCACCAAGAUGG |
| | UUCAUGACGACCGAGGCUGAUAAACCUGAUGCAGAUGCAAUGGCCGACGUCAUUAUUGACGAUGUG |
| | AGCCGCGAGAAGUCCAUGCGCGAGGAUCAUAAAUCUUUUGAUGAUGUCAUCCCGGCCAAAAAAAUA |
| | AUCGACUGGAAAGGAGCGAAUCCCGUGACCGUCAUAAACGAGUAUUGCCAGAUAACACGACGAGAU |
| | UGGAGCUUCAGGAUUGAGUCCGUUGGUCCGUCUAACUCUCCCACCUUCUAUGCGUGUGUUGACAUA |
| | GACGGUAGGGUCUUUGACAAAGCUGAUGGUAAAUCCAAGCGAGACGCCAAGAAUAAUGCAGCCAAA |
| | CUCGCCGUAGAUAAGUUGCUGGGAUACGUGAUUAUCCGAUUCUAAUAA |
| 23 | MSKIYIDERSNAEIVCEAIKTIGIEGATAAQLTROLNMEKREVNKALYDLQRSAMVYSSDDIPPRW |
| | FMTTEADKPDADAMADVIIDDVSREKSMREDHKSFDDVIPAKKIIDWKGANPVTVINEYCQITRRD |
| | WSFRIESVGPSNSPTFYACVDIDGRVFDKADGKSKRDAKNNAAKLAVDKLLGYVIIRF** |
| 24 | ATGACGACGCGGACAAAAGGCCGAGGCCACACTGCGGTCACAACCCAGAATGACAGGATGCCCGGT |
| | CCGGAATTGTCAGGCTGGATCAGCGAACAACTTATGACGGGGAGGATACCCGTAAGTGATATATTC |
| | TGCGACATAGAGAATAACCCGGGTCTTTGCCACGTAAGCCAGATGCAACAGACAAAACCGAACCCC |
| | AAAACGCGCAACAGTCAAACCCAGACCGACCCCATTTGTAACCATAGCTTCGAAGAGGTTGTTCAA |
| | ACTCTGGCAAGCCTGGCTACAGTCGTCCAACAACAGACAATAGCTTCCGAAAGCTTGGAGCAGCGC |
| | ATAACGTCTTTGGAAAACGGATTGAAACCAGTTTACGACATGGCGAAAACTATAAGCTCACTTAAC |
| | AGGGTCTGTGCGGAAATGGTCGCCAAGTACGACCTTCTCGTTATGACTACTGGCCGAGCGACCGCC |
| | ACTGCCGCAGCGACAGAAGCATATTGGGCAGAGCATGGACAGCCACCCCCAGGGCCTTCTTTGTAC |
| | GAGGAATCCACCATACGAGGAAAGATAGAAAGTAGAGATGAAACCGTGCCTCAGTCTGTGCGAGAG |
| | GCGTTTAACAATCTTGACTCTACAACATCATTGACAGAAGAAAACTTCGGTAAACCAGACATAAGC |
| | GCGAAGGACCTTCGAAATATCATGTACGACCACCTTCCGGGGTTCGGAACCGCGTTTCACCAACTG |
| | GTTCAAGTGATCTGTAAGCTCGGTAAAGATTCTAATAGTCTTGACATCATTCACGCCGAGTTTCAG |
| | GCCAGTCTGGCGGAGGGGGATTCACCACAGTGTGCACTTATACAAATTACCAAACGGGTGCCGATT |
| | TTCCAGGATGCCGCGCCCCCTGTTATTCACATTCGGTCCAGGGGCGATATCCCGAGAGCGTGCCAA |
| | AAGTCCCTGCGCCCTGTACCGCCCAGTCCAAAGATTGACCGAGGCTGGGTTTGTGTCTTCCAGTTG |
| | CAGGACGGAAAGACACTCGGGTTGAAGATATGATAA |
| 25 | AUGACGACGCGGACAAAAGGCCGAGGCCACACUGCGGUCACAACCCAGAAUGACAGGAUGCCCGGU |
| | CCGGAAUUGUCAGGCUGGAUCAGCGAACAACUUAUGACGGGGAGGAUACCCGUAAGUGAUAUAUUC |
| | UGCGACAUAGAGAAUAACCCGGGUCUUUGCCACGUAAGCCAGAUGCAACAGACAAAACCGAACCCC |
| | AAAACGCGCAACAGUCAAACCCAGACCGACCCCAUUUGUAACCAUAGCUUCGAAGAGGUUGUUCAA |
| | ACUCUGGCAAGCCUGGCUACAGUCGUCCAACAACAGACAAUAGCUUCCGAAAGCUUGGAGCAGCGC |
| | AUAACGUCUUUGGAAAACGGAUUGAAACCAGUUUACGACAUGGCGAAAACUAUAAGCUCACUUAAC |
| | AGGGUCUGUGCGGAAAUGGUCGCCAAGUACGACCUUCUCGUUAUGACUACUGGCCGAGCGACCGCC |
| | ACUGCCGCAGCGACAGAAGCAUAUUGGGCAGAGCAUGGACAGCCACCCCCAGGGCCUUCUUUGUAC |
| | GAGGAAUCCACCAUACGAGGAAAGAUAGAAAGUAGAGAUGAAACCGUGCCUCAGUCUGUGCGAGAG |
| | GCGUUUAACAAUCUUGACUCUACAACAUCAUUGACAGAAGAAAACUUCGGUAAACCAGACAUAAGC |
| | GCGAAGGACCUUCGAAAUAUCAUGUACGACCACCUUCCGGGGUUCGGAACCGCGUUUCACCAACUG |
| | GUUCAAGUGAUCUGUAAGCUCGGUAAAGAUUCUAAUAGUCUUGACAUCAUUCACGCCGAGUUUCAG |
| | GCCAGUCUGGCGGAGGGGGAUUCACCACAGUGUGCACUUAUACAAAUUACCAAACGGGUGCCGAUU |
| | UUCCAGGAUGCCGCGCCCCCUGUUAUUCACAUUCGGUCCAGGGGCGAUAUCCCGAGAGCGUGCCAA |
| | AAGUCCCUGCGCCCUGUACCGCCCAGUCCAAAGAUUGACCGAGGCUGGGUUUGUGUCUUCCAGUUG |
| | CAGGACGGAAAGACACUCGGGUUGAAGAUAUGAUAA |
| 26 | MTTRTKGRGHTAVTTQNDRMPGPELSGWISEQLMTGRIPVSDIFCDIENNPGLCHVSQMQQTKPNP |
| | KTRNSQTQTDPICNHSFEEVVQTLASLATVVQQQTIASESLEQRITSLENGLKPVYDMAKTISSLN |
| | RVCAEMVAKYDLLVMTTGRATATAAATEAYWAEHGQPPPGPSLYEESTIRGKIESRDETVPQSVRE |
| | AFNNLDSTTSLTEENFGKPDISAKDLRNIMYDHLPGFGTAFHQLVQVICKLGKDSNSLDIIHAEFQ |
| | ASLAEGDSPQCALIQITKRVPIFQDAAPPVIHIRSRGDIPRACQKSLRPVPPSPKIDRGWVCVFQL |
| | QDGKTLGLKI** |
| 27 | AUGCCGAAUAAUAACGGUAAGCAACAGAAGAAGAAAAAGGGUAAUGGACAACCCGUUAACCAACUU |
| | UGCCAGAUGCUGGGUAAGAUAAUUGCGCAACAAAACCAAUCAAGGGGGAAGGGGCCAGGGAAGAAG |
| | AAUAGAAAGAAAAAUCCCGAAAAACCCCAUUUUCCUUUGGCAACGGAGGACGAUGUACGACAUCAU |
| | UUCACCCCCAGCGAGAGGCAACUUUGUCUGAGUAGCAUUCAAACCGCCUUUAAUCAAGGCGCUGGG |
| | ACUUGCGCCCUUAGUGACUCUGGUCGAAUCUCAUACACCGUAGAAUUUAGUCUCCCGACCCAGCAU |
| | ACUGUUCGACUGAUCCGAGCCACCGCCUCUCCUUCUGCUUGAUAA |
| 28 | AUGGUUGCCCAUAAUCAGGUAGCUGCGGACAAUGCCGUCUCCACCGCGGCUGAACCUCGCCGGAGG |
| | CCCGAACCGAGUUCAAGUAGUUCCAGUAGUCCCGCCGCUCCUGCUAGGCCGAGACCCUGUCCCGCC |
| | GUUCCAGCCCCUGCACCAGGAGAUACACACUUUAGGACUUUCCGGAGUCACGCGGACUAUAGACGC |
| | AUCACACGCGCAUCUGCGUUGCUGGACGCGUGCGGUUUCUACUGGGGACCUCUCAGUGUACACGGG |
| | GCUCACGAAAGGCUUCGAGCGGAGCCCGUUGGUACGUUUCUUGUUCGAGACUCCAGGCAGCGAAAU |
| | UGCUUUUUUGCUCUCAGCGUCAAGAUGGCCUCCGGACCGACUUCAAUCAGAGUUCACUUCCAAGCA |
| | GGUAGGUUUCAUUUGGACGGAUCUCGCGAGAGUUUCGACUGCCUCUUUGAACUUCUGGAGCAUUAU |
| | GUUGCUGCGCCCAGGCGAAUGUUGGGUGCGCCCCUCCGCCAACGGAGGGUCCGACCGCUGCAAGAG |
| | UUGUGUAGGCAACGCAUUGUAGCUACCGUUGGUAGAGAAAACCUGGCUAGGAUUCCUCUUAACCCG |
| | GUGCUUCGGGAUUAUCUCUCCAGCUUCCCAUUCCAAAUAUGAUAA |
| 29 | AUGUUCACUCUCCAAGAUUCUUACGUUAAACAGAUGGUAAUGAGCUUGCGGGUUUCAGAGUUGCAA |
| | GUUCUUUUGGGUUACGCCGGACGAAAUAAACACGGGAGGAAACACGAGUUGCUUACAAAGGCUCUG |
| | CAUCUGCUUAAAGCGGGGUGCUCACCAGCCGUUCAGAUGAAAAUAAAAGAGCUGUAUCGCCGACGG |
| | UUUCCUCAAAAAAUCAUGACCCCGGCGGACUUGUCAAUUCCUAAUGUGCACAGUUCUCCCAUGCCC |
| | GCGACCCUGUCUCCCAGUACAAUCCCACAACUGACCUAUGAUGGCCACCCUGCGUCAAGUCCUCUC |
| | UUGCCGGUCUCACUUCUGGGUCCGAAGCAUGAGUUGGAGCUUCCUCAUCUCACAUCCGCGUUGCAC |
| | CCUGUCCAUCCUGACAUCAAAUUGCAAAAGCUUCCAUUCUACGACCUCCUGGACGAGCUUAUCAAG |
| | CCAACGAGCCUCGCGUCUGACAACUCUCAGCGAUUUAGGGAGACGUGCUUCGCGUUCGCACUGACC |
| | CCGCAACAGGUACAACAGAUCAGCUCGUCUAUGGAUAUAUCAGGCACGAAGUGCGACUUCACUGUC |
| | CAAGUACAGCUUCGGUUUUGCCUCUCAGAAACAUCCUGCCCUCAGGAGGAUCACUUCCCUCCGAAC |
| | CUUUGCGUCAAGGUGAACACGAAGCCAUGUUCUCUCCCGGGAUAUUUGCCUCCUACCAAGAAUGGC |
| | GUUGAGCCUAAACGCCCCUCCCGACCGAUCAACAUUACCUCAUUGGUCCGCUUGUCAACUACCGUC |
| | CCGAAUACUAUUGUGGUGAGUUGGACAGCAGAGAUAGGUCGAAAUUACUCAAUGGCGGUUUAUCUG |
| | GUAAAGCAGCUGAGCAGCACUGUCUUGCUGCAGAGACUCCGCGCCAAGGGUAUACGAAACCCGGAU |
| | CACUCCCGAGCAUUGAUCAAGGAAAAACUCACAGCGGAUCCAGACAGCGAGAUAGCCACCACCUCC |
| | UUGCGGGUUAGCCUCUUGUGUCCGUUGGGUAAGAUGCGACUGACCAUUCCUUGCAGGGCACUCACG |
| | UGUUCACAUCUCCAAUGUUUUGACGCCACGUUGUAUAUACAGAUGAAUGAAAAGAAACCGACUUGG |
| | GUUUGUCCAGUGUGUGAUAAGAAAGCACCAUAUGAACAUCUUAUCAUAGAUGGCCUGUUUAUGGAG |
| | AUUCUGAAGUAUUGCACUGAUUGUGAUGAAAUCCAGUUCAAAGAAGAUGGUACGUGGGCUCCCAUG |
| | AGAAGUAAAAAGGAGGUGCAAGAAGUUAGCGCGAGCUACAACGGGGUGGAUGGGUGCUUGAGUUCC |
| | ACUUUGGAACACCAAGUGGCCUCCCAUCACCAGUCCAGCAAUAAGAACAAGAAGGUCGAAGUAAUC |
| | GACCUGACUAUUGAUUCAUCCUCCGACGAGGAGGAAGAGGAACCUAGCGCCAAGCGCACAUGCCCU |
| | UCACUUUCUCCCACGUCACCUUUGAACAAUAAGGGAAUUUUGUCUUUGCCCCACCAAGCUUCCCCG |
| | GUGUCCAGGACACCAAGCCUCCCCGCCGUUGAUACCUCCUAUAUAAAUACGUCACUGAUUCAGGAC |
| | UAUCGCCACCCAUUUCACAUGACCCCUAUGCCCUAUGAUCUGCAAGGACUGGACUUCUUCCCGUUU |
| | UUGUCCGGAGAUAAUCAGCAUUACAACACUAGUCUUCUUGCAGCCGCGGCAGCGGCGGUAAGCGAU |
| | GACCAGGAUCUUCUGCACUCUAGUAGAUUUUUCCCGUACACCUCCUCACAAAUGUUUCUGGAUCAG |
| | CUUAGUGCUGGCGGAAGCACUUCUCUCCCGACUACCAACGGCAGUUCAUCCGGCUCAAACUCUAGC |
| | CUCGUGUCUAGUAAUAGUCUUAGAGAGAGUCAUUCACAUACCGUUACGAAUAGGUCUUCAACCGAU |
| | ACCGCGUCAAUCUUUGGCAUUAUUCCUGAUAUAAUAUCACUCGAUUGAUAA |
| 30 | ATGCCGAATAATAACGGTAAGCAACAGAAGAAGAAAAAGGGTAATGGACAACCCGTTAACCAACTT |
| | TGCCAGATGCTGGGTAAGATAATTGCGCAACAAAACCAATCAAGGGGGAAGGGGCCAGGGAAGAAG |
| | AATAGAAAGAAAAATCCCGAAAAACCCCATTTTCCTTTGGCAACGGAGGACGATGTACGACATCAT |
| | TTCACCCCCAGCGAGAGGCAACTTTGTCTGAGTAGCATTCAAACCGCCTTTAATCAAGGCGCTGGG |
| | ACTTGCGCCCTTAGTGACTCTGGTCGAATCTCATACACCGTAGAATTTAGTCTCCCGACCCAGCAT |
| | ACTGTTCGACTGATCCGAGCCACCGCCTCTCCTTCTGCTTGATAA |
| 31 | MPNNNGKQQKKKKGNGQPVNQLCQMLGKIIAQQNQSRGKGPGKKNRKKNPEKPHFPLATEDDVRHH |
| | FTPSERQLCLSSIQTAFNOGAGTCALSDSGRISYTVEFSLPTQHTVRLIRATASPSA** |
| 32 | ATAGGCGGCGCATGAGAGAAGCCCAGACCAATTACCTACCCAAAATGGAGAAAGTTCACGTTGACA |
| | TCGAGGAAGACAGCCCATTCCTCAGAGCTTTGCAGCGGAGCTTCCCGCAGTTTGAGGTAGAAGCCA |
| | AGCAGGTCACTGATAATGACCATGCTAATGCCAGAGCGTTTTCGCATCTGGCTTCAAAACTGATCG |
| | AAACGGAGGTGGACCCATCCGACACGATCCTTGACATTGGAAGTGCGCCCGCCCGCAGAATGTATT |
| | CTAAGCACAAGTATCATTGTATCTGTCCGATGAGATGTGCGGAAGATCCGGACAGATTGTATAAGT |
| | ATGCAACTAAGCTGAAGAAAAACTGTAAGGAAATAACTGATAAGGAATTGGACAAGAAAATGAAGG |
| | AGCTCGCCGCCGTCATGAGCGACCCTGACCTGGAAACTGAGACTATGTGCCTCCACGACGACGAGT |
| | CGTGTCGCTACGAAGGGCAAGTCGCTGTTTACCAGGATGTATACGCGGTTGACGGACCGACAAGTC |
| | TCTATCACCAAGCCAATAAGGGAGTTAGAGTCGCCTACTGGATAGGCTTTGACACCACCCCTTTTA |
| | TGTTTAAGAACTTGGCTGGAGCATATCCATCATACTCTACCAACTGGGCCGACGAAACCGTGTTAA |
| | CGGCTCGTAACATAGGCCTATGCAGCTCTGACGTTATGGAGCGGTCACGTAGAGGGATGTCCATTC |
| | TTAGAAAGAAGTATTTGAAACCATCCAACAATGTTCTATTCTCTGTTGGCTCGACCATCTACCACG |
| | AGAAGAGGGACTTACTGAGGAGCTGGCACCTGCCGTCTGTATTTCACTTACGTGGCAAGCAAAATT |
| | ACACATGTCGGTGTGAGACTATAGTTAGTTGCGACGGGTACGTCGTTAAAAGAATAGCTATCAGTC |
| | CAGGCCTGTATGGGAAGCCTTCAGGCTATGCTGCTACGATGCACCGCGAGGGATTCTTGTGCTGCA |
| | AAGTGACAGACACATTGAACGGGGAGAGGGTCTCTTTTCCCGTGTGCACGTATGTGCCAGCTACAT |
| | TGTGTGACCAAATGACTGGCATACTGGCAACAGATGTCAGTGCGGACGACGCGCAAAAACTGCTGG |
| | TTGGGCTCAACCAGCGTATAGTCGTCAACGGTCGCACCCAGAGAAACACCAATACCATGAAAAATT |
| | ACCTTTTGCCCGTAGTGGCCCAGGCATTTGCTAGGTGGGCAAAGGAATATAAGGAAGATCAAGAAG |
| | ATGAAAGGCCACTAGGACTACGAGATAGACAGTTAGTCATGGGGTGTTGTTGGGCTTTTAGAAGGC |
| | ACAAGATAACATCTATTTATAAGCGCCCGGATACCCAAACCATCATCAAAGTGAACAGCGATTTCC |
| | ACTCATTCGTGCTGCCCAGGATAGGCAGTAACACATTGGAGATCGGGCTGAGAACAAGAATCAGGA |
| | AAATGTTAGAGGAGCACAAGGAGCCGTCACCTCTCATTACCGCCGAGGACGTACAAGAAGCTAAGT |
| | GCGCAGCCGATGAGGCTAAGGAGGTGCGTGAAGCCGAGGAGTTGCGCGCAGCTCTACCACCTTTGG |
| | CAGCTGATGTTGAGGAGCCCACTCTGGAAGCCGATGTCGACTTGATGTTACAAGAGGCTGGGGCCG |
| | GCTCAGTGGAGACACCTCGTGGCTTGATAAAGGTTACCAGCTACGATGGCGAGGACAAGATCGGCT |
| | CTTACGCTGTGCTTTCTCCGCAGGCTGTACTCAAGAGTGAAAAATTATCTTGCATCCACCCTCTCG |
| | CTGAACAAGTCATAGTGATAACACACTCTGGCCGAAAAGGGCGTTATGCCGTGGAACCATACCATG |
| | GTAAAGTAGTGGTGCCAGAGGGACATGCAATACCCGTCCAGGACTTTCAAGCTCTGAGTGAAAGTG |
| | CCACCATTGTGTACAACGAACGTGAGTTCGTAAACAGGTACCTGCACCATATTGCCACACATGGAG |
| | GAGCGCTGAACACTGATGAAGAATATTACAAAACTGTCAAGCCCAGCGAGCACGACGGCGAATACC |
| | TGTACGACATCGACAGGAAACAGTGCGTCAAGAAAGAACTAGTCACTGGGCTAGGGCTCACAGGCG |
| | AGCTGGTGGATCCTCCCTTCCATGAATTCGCCTACGAGAGTCTGAGAACACGACCAGCCGCTCCTT |
| | ACCAAGTACCAACCATAGGGGTGTATGGCGTGCCAGGATCAGGCAAGTCTGGCATCATTAAAAGCG |
| | CAGTCACCAAAAAAGATCTAGTGGTGAGCGCCAAGAAAGAAAACTGTGCAGAAATTATAAGGGACG |
| | TCAAGAAAATGAAAGGGCTGGACGTCAATGCCAGAACTGTGGACTCAGTGCTCTTGAATGGATGCA |
| | AACACCCCGTAGAGACCCTGTATATTGACGAAGCTTTTGCTTGTCATGCAGGTACTCTCAGAGCGC |
| | TCATAGCCATTATAAGACCTAAAAAGGCAGTGCTCTGCGGGGATCCCAAACAGTGCGGTTTTTTTA |
| | ACATGATGTGCCTGAAAGTGCATTTTAACCACGAGATTTGCACACAAGTCTTCCACAAAAGCATCT |
| | CTCGCCGTTGCACTAAATCTGTGACTTCGGTCGTCTCAACCTTGTTTTACGACAAAAAAATGAGAA |
| | CGACGAATCCGAAAGAGACTAAGATTGTGATTGACACTACCGGCAGTACCAAACCTAAGCAGGACG |
| | ATCTCATTCTCACTTGTTTCAGAGGGTGGGTGAAGCAGTTGCAAATAGATTACAAAGGCAACGAAA |
| | TAATGACGGCAGCTGCCTCTCAAGGGCTGACCCGTAAAGGTGTGTATGCCGTTCGGTACAAGGTGA |
| | ATGAAAATCCTCTGTACGCACCCACCTCAGAACATGTGAACGTCCTACTGACCCGCACGGAGGACC |
| | GCATCGTGTGGAAAACACTAGCCGGCGACCCATGGATAAAAACACTGACTGCCAAGTACCCTGGGA |
| | ATTTCACTGCCACGATAGAGGAGTGGCAAGCAGAGCATGATGCCATCATGAGGCACATCTTGGAGA |
| | GACCGGACCCTACCGACGTCTTCCAGAATAAGGCAAACGTGTGTTGGGCCAAGGCTTTAGTGCCGG |
| | TGCTGAAGACCGCTGGCATAGACATGACCACTGAACAATGGAACACTGTGGATTATTTTGAAACGG |
| | ACAAAGCTCACTCAGCAGAGATAGTATTGAACCAACTATGCGTGAGGTTCTTTGGACTCGATCTGG |
| | ACTCCGGTCTATTTTCTGCACCCACTGTTCCGTTATCCATTAGGAATAATCACTGGGATAACTCCC |
| | CGTCGCCTAACATGTACGGGCTGAATAAAGAAGTGGTCCGTCAGCTCTCTCGCAGGTACCCACAAC |
| | TGCCTCGGGCAGTTGCCACTGGAAGAGTCTATGACATGAACACTGGTACACTGCGCAATTATGATC |
| | CGCGCATAAACCTAGTACCTGTAAACAGAAGACTGCCTCATGCTTTAGTCCTCCACCATAATGAAC |
| | ACCCACAGAGTGACTTTTCTTCATTCGTCAGCAAATTGAAGGGCAGAACTGTCCTGGTGGTCGGGG |
| | AAAAGTTGTCCGTCCCAGGCAAAATGGTTGACTGGTTGTCAGACCGGCCTGAGGCTACCTTCAGAG |
| | CTCGGCTGGATTTAGGCATCCCAGGTGATGTGCCCAAATATGACATAATATTTGTTAATGTGAGGA |
| | CCCCATATAAATACCATCACTATCAGCAGTGTGAAGACCATGCCATTAAGCTTAGCATGTTGACCA |
| | AGAAAGCTTGTCTGCATCTGAATCCCGGCGGAACCTGTGTCAGCATAGGTTATGGTTACGCTGACA |
| | GGGCCAGCGAAAGCATCATTGGTGCTATAGCGCGGCAGTTCAAGTTTTCCCGGGTATGCAAACCGA |
| | AATCCTCACTTGAAGAGACGGAAGTTCTGTTTGTATTCATTCGGTACGATCGCAAGGCCCGTACGC |
| | ACAATCCTTACAAGCTTTCATCAACCTTGACCAACATTTATACAGGTTCCAGACTCCACGAAGCCG |
| | GATGTGCACCCTCATATCATGTGGTGCGAGGGGATATTGCCACGGCCACCGAAGGAGTGATTATAA |
| | ATGCTGCTAACAGCAAAGGACAACCTGGCGGAGGGGTGTGCGGAGCGCTGTATAAGAAATTCCCGG |
| | AAAGCTTCGATTTACAGCCGATCGAAGTAGGAAAAGCGCGACTGGTCAAAGGTGCAGCTAAACATA |
| | TCATTCATGCCGTAGGACCAAACTTCAACAAAGTTTCGGAGGTTGAAGGTGACAAACAGTTGGCAG |
| | AGGCTTATGAGTCCATCGCTAAGATTGTCAACGATAACAATTACAAGTCAGTAGCGATTCCACTGT |
| | TGTCCACCGGCATCTTTTCCGGGAACAAAGATCGACTAACCCAATCATTGAACCATTTGCTGACAG |
| | CTTTAGACACCACTGATGCAGATGTAGCCATATACTGCAGGGACAAGAAATGGGAAATGACTCTCA |
| | AGGAAGCAGTGGCTAGGAGAGAAGCAGTGGAGGAGATATGCATATCCGACGACTCTTCAGTGACAG |
| | AACCTGATGCAGAGCTGGTGAGGGTGCATCCGAAGAGTTCTTTGGCTGGAAGGAAGGGCTACAGCA |
| | CAAGCGATGGCAAAACTTTCTCATATTTGGAAGGGACCAAGTTTCACCAGGCGGCCAAGGATATAG |
| | CAGAAATTAATGCCATGTGGCCCGTTGCAACGGAGGCCAATGAGCAGGTATGCATGTATATCCTCG |
| | GAGAAAGCATGAGCAGTATTAGGTCGAAATGCCCCGTCGAAGAGTCGGAAGCCTCCACACCACCTA |
| | GCACGCTGCCTTGCTTGTGCATCCATGCCATGACTCCAGAAAGAGTACAGCGCCTAAAAGCCTCAC |
| | GTCCAGAACAAATTACTGTGTGCTCATCCTTTCCATTGCCGAAGTATAGAATCACTGGTGTGCAGA |
| | AGATCCAATGCTCCCAGCCTATATTGTTCTCACCGAAAGTGCCTGCGTATATTCATCCAAGGAAGT |
| | ATCTCGTGGAAACACCACCGGTAGACGAGACTCCGGAGCCATCGGCAGAGAACCAATCCACAGAGG |
| | GGACACCTGAACAACCACCACTTATAACCGAGGATGAGACCAGGACTAGAACGCCTGAGCCGATCA |
| | TCATCGAAGAGGAAGAAGAGGATAGCATAAGTTTGCTGTCAGATGGCCCGACCCACCAGGTGCTGC |
| | AAGTCGAGGCAGACATTCACGGGCCGCCCTCTGTATCTAGCTCATCCTGGTCCATTCCTCATGCAT |
| | CCGACTTTGATGTGGACAGTTTATCCATACTTGACACCCTGGAGGGAGCTAGCGTGACCAGCGGGG |
| | CAACGTCAGCCGAGACTAACTCTTACTTCGCAAAGAGTATGGAGTTTCTGGCGCGACCGGTGCCTG |
| | CGCCTCGAACAGTATTCAGGAACCCTCCACATCCCGCTCCGCGCACAAGAACACCGTCACTTGCAC |
| | CCAGCAGGGCCTGCTCGAGAACCAGCCTAGTTTCCACCCCGCCAGGCGTGAATAGGGTGATCACTA |
| | GAGAGGAGCTCGAGGCGCTTACCCCGTCACGCACTCCTAGCAGGTCGGTCTCGAGAACCAGCCTGG |
| | TCTCCAACCCGCCAGGCGTAAATAGGGTGATTACAAGAGAGGAGTTTGAGGCGTTCGTAGCACAAC |
| | AACAATGACGGTTTGATGCGGGTGCATACATCTTTTCCTCCGACACCGGTCAAGGGCATTTACAAC |
| | AAAAATCAGTAAGGCAAACGGTGCTATCCGAAGTGGTGTTGGAGAGGACCGAATTGGAGATTTCGT |
| | ATGCCCCGCGCCTCGACCAAGAAAAAGAAGAATTACTACGCAAGAAATTACAGTTAAATCCCACAC |
| | CTGCTAACAGAAGCAGATACCAGTCCAGGAAGGTGGAGAACATGAAAGCCATAACAGCTAGACGTA |
| | TTCTGCAAGGCCTAGGGCATTATTTGAAGGCAGAAGGAAAAGTGGAGTGCTACCGAACCCTGCATC |
| | CTGTTCCTTTGTATTCATCTAGTGTGAACCGTGCCTTTTCAAGCCCCAAGGTCGCAGTGGAAGCCT |
| | GTAACGCCATGTTGAAAGAGAACTTTCCGACTGTGGCTTCTTACTGTATTATTCCAGAGTACGATG |
| | CCTATTTGGACATGGTTGACGGAGCTTCATGCTGCTTAGACACTGCCAGTTTTTGCCCTGCAAAGC |
| | TGCGCAGCTTTCCAAAGAAACACTCCTATTTGGAACCCACAATACGATCGGCAGTGCCTTCAGCGA |
| | TCCAGAACACGCTCCAGAACGTCCTGGCAGCTGCCACAAAAAGAAATTGCAATGTCACGCAAATGA |
| | GAGAATTGCCCGTATTGGATTCGGCGGCCTTTAATGTGGAATGCTTCAAGAAATATGCGTGTAATA |
| | ATGAATATTGGGAAACGTTTAAAGAAAACCCCATCAGGCTTACTGAAGAAAACGTGGTAAATTACA |
| | TTACCAAATTAAAAGGACCAAAAGCTGCTGCTCTTTTTGCGAAGACACATAATTTGAATATGTTGC |
| | AGGACATACCAATGGACAGGTTTGTAATGGACTTAAAGAGAGACGTGAAAGTGACTCCAGGAACAA |
| | AACATACTGAAGAACGGCCCAAGGTACAGGTGATCCAGGCTGCCGATCCGCTAGCAACAGCGTATC |
| | TGTGCGGAATCCACCGAGAGCTGGTTAGGAGATTAAATGCGGTCCTGCTTCCGAACATTCATACAC |
| | TGTTTGATATGTCGGCTGAAGACTTTGACGCTATTATAGCCGAGCACTTCCAGCCTGGGGATTGTG |
| | TTCTGGAAACTGACATCGCGTCGTTTGATAAAAGTGAGGACGACGCCATGGCTCTGACCGCGTTAA |
| | TGATTCTGGAAGACTTAGGTGTGGACGCAGAGCTGTTGACGCTGATTGAGGCGGCTTTCGGCGAAA |
| | TTTCATCAATACATTTGCCCACTAAAACTAAATTTAAATTCGGAGCCATGATGAAATCTGGAATGT |
| | TCCTCACACTGTTTGTGAACACAGTCATTAACATTGTAATCGCAAGCAGAGTGTTGAGAGAACGGC |
| | TAACCGGATCACCATGTGCAGCATTCATTGGAGATGACAATATCGTGAAAGGAGTCAAATCGGACA |
| | AATTAATGGCAGACAGGTGCGCCACCTGGTTGAATATGGAAGTCAAGATTATAGATGCTGTGGTGG |
| | GCGAGAAAGCGCCTTATTTCTGTGGAGGGTTTATTTTGTGTGACTCCGTGACCGGCACAGCGTGCC |
| | GTGTGGCAGACCCCCTAAAAAGGCTGTTTAAGCTTGGCAAACCTCTGGCAGCAGACGATGAACATG |
| | ATGATGACAGGAGAAGGGCATTGCATGAAGAGTCAACACGCTGGAACCGAGTGGGTATTCTTTCAG |
| | AGCTGTGCAAGGCAGTAGAATCAAGGTATGAAACCGTAGGAACTTCCATCATAGTTATGGCCATGA |
| | CTACTCTAGCTAGCAGTGTTAAATCATTCAGCTACCTGAGAGGGGCCCCTATAACTCTCTACGGCT |
| | AACCTGAATGGACTACGACATAGTCTAGTCCGCCAAG |
| 33 | AUAGGCGGCGCAUGAGAGAAGCCCAGACCAAUUACCUACCCAAAAUGGAGAAAGUUCACGUUGACA |
| | UCGAGGAAGACAGCCCAUUCCUCAGAGCUUUGCAGCGGAGCUUCCCGCAGUUUGAGGUAGAAGCCA |
| | AGCAGGUCACUGAUAAUGACCAUGCUAAUGCCAGAGCGUUUUCGCAUCUGGCUUCAAAACUGAUCG |
| | AAACGGAGGUGGACCCAUCCGACACGAUCCUUGACAUUGGAAGUGCGCCCGCCCGCAGAAUGUAUU |
| | CUAAGCACAAGUAUCAUUGUAUCUGUCCGAUGAGAUGUGCGGAAGAUCCGGACAGAUUGUAUAAGU |
| | AUGCAACUAAGCUGAAGAAAAACUGUAAGGAAAUAACUGAUAAGGAAUUGGACAAGAAAAUGAAGG |
| | AGCUCGCCGCCGUCAUGAGCGACCCUGACCUGGAAACUGAGACUAUGUGCCUCCACGACGACGAGU |
| | CGUGUCGCUACGAAGGGCAAGUCGCUGUUUACCAGGAUGUAUACGCGGUUGACGGACCGACAAGUC |
| | UCUAUCACCAAGCCAAUAAGGGAGUUAGAGUCGCCUACUGGAUAGGCUUUGACACCACCCCUUUUA |
| | UGUUUAAGAACUUGGCUGGAGCAUAUCCAUCAUACUCUACCAACUGGGCCGACGAAACCGUGUUAA |
| | CGGCUCGUAACAUAGGCCUAUGCAGCUCUGACGUUAUGGAGCGGUCACGUAGAGGGAUGUCCAUUC |
| | UUAGAAAGAAGUAUUUGAAACCAUCCAACAAUGUUCUAUUCUCUGUUGGCUCGACCAUCUACCACG |
| | AGAAGAGGGACUUACUGAGGAGCUGGCACCUGCCGUCUGUAUUUCACUUACGUGGCAAGCAAAAUU |
| | ACACAUGUCGGUGUGAGACUAUAGUUAGUUGCGACGGGUACGUCGUUAAAAGAAUAGCUAUCAGUC |
| | CAGGCCUGUAUGGGAAGCCUUCAGGCUAUGCUGCUACGAUGCACCGCGAGGGAUUCUUGUGCUGCA |
| | AAGUGACAGACACAUUGAACGGGGAGAGGGUCUCUUUUCCCGUGUGCACGUAUGUGCCAGCUACAU |
| | UGUGUGACCAAAUGACUGGCAUACUGGCAACAGAUGUCAGUGCGGACGACGCGCAAAAACUGCUGG |
| | UUGGGCUCAACCAGCGUAUAGUCGUCAACGGUCGCACCCAGAGAAACACCAAUACCAUGAAAAAUU |
| | ACCUUUUGCCCGUAGUGGCCCAGGCAUUUGCUAGGUGGGCAAAGGAAUAUAAGGAAGAUCAAGAAG |
| | AUGAAAGGCCACUAGGACUACGAGAUAGACAGUUAGUCAUGGGGUGUUGUUGGGCUUUUAGAAGGC |
| | ACAAGAUAACAUCUAUUUAUAAGCGCCCGGAUACCCAAACCAUCAUCAAAGUGAACAGCGAUUUCC |
| | ACUCAUUCGUGCUGCCCAGGAUAGGCAGUAACACAUUGGAGAUCGGGCUGAGAACAAGAAUCAGGA |
| | AAAUGUUAGAGGAGCACAAGGAGCCGUCACCUCUCAUUACCGCCGAGGACGUACAAGAAGCUAAGU |
| | GCGCAGCCGAUGAGGCUAAGGAGGUGCGUGAAGCCGAGGAGUUGCGCGCAGCUCUACCACCUUUGG |
| | CAGCUGAUGUUGAGGAGCCCACUCUGGAAGCCGAUGUCGACUUGAUGUUACAAGAGGCUGGGGCCG |
| | GCUCAGUGGAGACACCUCGUGGCUUGAUAAAGGUUACCAGCUACGAUGGCGAGGACAAGAUCGGCU |
| | CUUACGCUGUGCUUUCUCCGCAGGCUGUACUCAAGAGUGAAAAAUUAUCUUGCAUCCACCCUCUCG |
| | CUGAACAAGUCAUAGUGAUAACACACUCUGGCCGAAAAGGGCGUUAUGCCGUGGAACCAUACCAUG |
| | GUAAAGUAGUGGUGCCAGAGGGACAUGCAAUACCCGUCCAGGACUUUCAAGCUCUGAGUGAAAGUG |
| | CCACCAUUGUGUACAACGAACGUGAGUUCGUAAACAGGUACCUGCACCAUAUUGCCACACAUGGAG |
| | GAGCGCUGAACACUGAUGAAGAAUAUUACAAAACUGUCAAGCCCAGCGAGCACGACGGCGAAUACC |
| | UGUACGACAUCGACAGGAAACAGUGCGUCAAGAAAGAACUAGUCACUGGGCUAGGGCUCACAGGCG |
| | AGCUGGUGGAUCCUCCCUUCCAUGAAUUCGCCUACGAGAGUCUGAGAACACGACCAGCCGCUCCUU |
| | ACCAAGUACCAACCAUAGGGGUGUAUGGCGUGCCAGGAUCAGGCAAGUCUGGCAUCAUUAAAAGCG |
| | CAGUCACCAAAAAAGAUCUAGUGGUGAGCGCCAAGAAAGAAAACUGUGCAGAAAUUAUAAGGGACG |
| | UCAAGAAAAUGAAAGGGCUGGACGUCAAUGCCAGAACUGUGGACUCAGUGCUCUUGAAUGGAUGCA |
| | AACACCCCGUAGAGACCCUGUAUAUUGACGAAGCUUUUGCUUGUCAUGCAGGUACUCUCAGAGCGC |
| | UCAUAGCCAUUAUAAGACCUAAAAAGGCAGUGCUCUGCGGGGAUCCCAAACAGUGCGGUUUUUUUA |
| | ACAUGAUGUGCCUGAAAGUGCAUUUUAACCACGAGAUUUGCACACAAGUCUUCCACAAAAGCAUCU |
| | CUCGCCGUUGCACUAAAUCUGUGACUUCGGUCGUCUCAACCUUGUUUUACGACAAAAAAAUGAGAA |
| | CGACGAAUCCGAAAGAGACUAAGAUUGUGAUUGACACUACCGGCAGUACCAAACCUAAGCAGGACG |
| | AUCUCAUUCUCACUUGUUUCAGAGGGUGGGUGAAGCAGUUGCAAAUAGAUUACAAAGGCAACGAAA |
| | UAAUGACGGCAGCUGCCUCUCAAGGGCUGACCCGUAAAGGUGUGUAUGCCGUUCGGUACAAGGUGA |
| | AUGAAAAUCCUCUGUACGCACCCACCUCAGAACAUGUGAACGUCCUACUGACCCGCACGGAGGACC |
| | GCAUCGUGUGGAAAACACUAGCCGGCGACCCAUGGAUAAAAACACUGACUGCCAAGUACCCUGGGA |
| | AUUUCACUGCCACGAUAGAGGAGUGGCAAGCAGAGCAUGAUGCCAUCAUGAGGCACAUCUUGGAGA |
| | GACCGGACCCUACCGACGUCUUCCAGAAUAAGGCAAACGUGUGUUGGGCCAAGGCUUUAGUGCCGG |
| | UGCUGAAGACCGCUGGCAUAGACAUGACCACUGAACAAUGGAACACUGUGGAUUAUUUUGAAACGG |
| | ACAAAGCUCACUCAGCAGAGAUAGUAUUGAACCAACUAUGCGUGAGGUUCUUUGGACUCGAUCUGG |
| | ACUCCGGUCUAUUUUCUGCACCCACUGUUCCGUUAUCCAUUAGGAAUAAUCACUGGGAUAACUCCC |
| | CGUCGCCUAACAUGUACGGGCUGAAUAAAGAAGUGGUCCGUCAGCUCUCUCGCAGGUACCCACAAC |
| | UGCCUCGGGCAGUUGCCACUGGAAGAGUCUAUGACAUGAACACUGGUACACUGCGCAAUUAUGAUC |
| | CGCGCAUAAACCUAGUACCUGUAAACAGAAGACUGCCUCAUGCUUUAGUCCUCCACCAUAAUGAAC |
| | ACCCACAGAGUGACUUUUCUUCAUUCGUCAGCAAAUUGAAGGGCAGAACUGUCCUGGUGGUCGGGG |
| | AAAAGUUGUCCGUCCCAGGCAAAAUGGUUGACUGGUUGUCAGACCGGCCUGAGGCUACCUUCAGAG |
| | CUCGGCUGGAUUUAGGCAUCCCAGGUGAUGUGCCCAAAUAUGACAUAAUAUUUGUUAAUGUGAGGA |
| | CCCCAUAUAAAUACCAUCACUAUCAGCAGUGUGAAGACCAUGCCAUUAAGCUUAGCAUGUUGACCA |
| | AGAAAGCUUGUCUGCAUCUGAAUCCCGGCGGAACCUGUGUCAGCAUAGGUUAUGGUUACGCUGACA |
| | GGGCCAGCGAAAGCAUCAUUGGUGCUAUAGCGCGGCAGUUCAAGUUUUCCCGGGUAUGCAAACCGA |
| | AAUCCUCACUUGAAGAGACGGAAGUUCUGUUUGUAUUCAUUCGGUACGAUCGCAAGGCCCGUACGC |
| | ACAAUCCUUACAAGCUUUCAUCAACCUUGACCAACAUUUAUACAGGUUCCAGACUCCACGAAGCCG |
| | GAUGUGCACCCUCAUAUCAUGUGGUGCGAGGGGAUAUUGCCACGGCCACCGAAGGAGUGAUUAUAA |
| | AUGCUGCUAACAGCAAAGGACAACCUGGCGGAGGGGUGUGCGGAGCGCUGUAUAAGAAAUUCCCGG |
| | AAAGCUUCGAUUUACAGCCGAUCGAAGUAGGAAAAGCGCGACUGGUCAAAGGUGCAGCUAAACAUA |
| | UCAUUCAUGCCGUAGGACCAAACUUCAACAAAGUUUCGGAGGUUGAAGGUGACAAACAGUUGGCAG |
| | AGGCUUAUGAGUCCAUCGCUAAGAUUGUCAACGAUAACAAUUACAAGUCAGUAGCGAUUCCACUGU |
| | UGUCCACCGGCAUCUUUUCCGGGAACAAAGAUCGACUAACCCAAUCAUUGAACCAUUUGCUGACAG |
| | CUUUAGACACCACUGAUGCAGAUGUAGCCAUAUACUGCAGGGACAAGAAAUGGGAAAUGACUCUCA |
| | AGGAAGCAGUGGCUAGGAGAGAAGCAGUGGAGGAGAUAUGCAUAUCCGACGACUCUUCAGUGACAG |
| | AACCUGAUGCAGAGCUGGUGAGGGUGCAUCCGAAGAGUUCUUUGGCUGGAAGGAAGGGCUACAGCA |
| | CAAGCGAUGGCAAAACUUUCUCAUAUUUGGAAGGGACCAAGUUUCACCAGGCGGCCAAGGAUAUAG |
| | CAGAAAUUAAUGCCAUGUGGCCCGUUGCAACGGAGGCCAAUGAGCAGGUAUGCAUGUAUAUCCUCG |
| | GAGAAAGCAUGAGCAGUAUUAGGUCGAAAUGCCCCGUCGAAGAGUCGGAAGCCUCCACACCACCUA |
| | GCACGCUGCCUUGCUUGUGCAUCCAUGCCAUGACUCCAGAAAGAGUACAGCGCCUAAAAGCCUCAC |
| | GUCCAGAACAAAUUACUGUGUGCUCAUCCUUUCCAUUGCCGAAGUAUAGAAUCACUGGUGUGCAGA |
| | AGAUCCAAUGCUCCCAGCCUAUAUUGUUCUCACCGAAAGUGCCUGCGUAUAUUCAUCCAAGGAAGU |
| | AUCUCGUGGAAACACCACCGGUAGACGAGACUCCGGAGCCAUCGGCAGAGAACCAAUCCACAGAGG |
| | GGACACCUGAACAACCACCACUUAUAACCGAGGAUGAGACCAGGACUAGAACGCCUGAGCCGAUCA |
| | UCAUCGAAGAGGAAGAAGAGGAUAGCAUAAGUUUGCUGUCAGAUGGCCCGACCCACCAGGUGCUGC |
| | AAGUCGAGGCAGACAUUCACGGGCCGCCCUCUGUAUCUAGCUCAUCCUGGUCCAUUCCUCAUGCAU |
| | CCGACUUUGAUGUGGACAGUUUAUCCAUACUUGACACCCUGGAGGGAGCUAGCGUGACCAGCGGGG |
| | CAACGUCAGCCGAGACUAACUCUUACUUCGCAAAGAGUAUGGAGUUUCUGGCGCGACCGGUGCCUG |
| | CGCCUCGAACAGUAUUCAGGAACCCUCCACAUCCCGCUCCGCGCACAAGAACACCGUCACUUGCAC |
| | CCAGCAGGGCCUGCUCGAGAACCAGCCUAGUUUCCACCCCGCCAGGCGUGAAUAGGGUGAUCACUA |
| | GAGAGGAGCUCGAGGCGCUUACCCCGUCACGCACUCCUAGCAGGUCGGUCUCGAGAACCAGCCUGG |
| | UCUCCAACCCGCCAGGCGUAAAUAGGGUGAUUACAAGAGAGGAGUUUGAGGCGUUCGUAGCACAAC |
| | AACAAUGACGGUUUGAUGCGGGUGCAUACAUCUUUUCCUCCGACACCGGUCAAGGGCAUUUACAAC |
| | AAAAAUCAGUAAGGCAAACGGUGCUAUCCGAAGUGGUGUUGGAGAGGACCGAAUUGGAGAUUUCGU |
| | AUGCCCCGCGCCUCGACCAAGAAAAAGAAGAAUUACUACGCAAGAAAUUACAGUUAAAUCCCACAC |
| | CUGCUAACAGAAGCAGAUACCAGUCCAGGAAGGUGGAGAACAUGAAAGCCAUAACAGCUAGACGUA |
| | UUCUGCAAGGCCUAGGGCAUUAUUUGAAGGCAGAAGGAAAAGUGGAGUGCUACCGAACCCUGCAUC |
| | CUGUUCCUUUGUAUUCAUCUAGUGUGAACCGUGCCUUUUCAAGCCCCAAGGUCGCAGUGGAAGCCU |
| | GUAACGCCAUGUUGAAAGAGAACUUUCCGACUGUGGCUUCUUACUGUAUUAUUCCAGAGUACGAUG |
| | CCUAUUUGGACAUGGUUGACGGAGCUUCAUGCUGCUUAGACACUGCCAGUUUUUGCCCUGCAAAGC |
| | UGCGCAGCUUUCCAAAGAAACACUCCUAUUUGGAACCCACAAUACGAUCGGCAGUGCCUUCAGCGA |
| | UCCAGAACACGCUCCAGAACGUCCUGGCAGCUGCCACAAAAAGAAAUUGCAAUGUCACGCAAAUGA |
| | GAGAAUUGCCCGUAUUGGAUUCGGCGGCCUUUAAUGUGGAAUGCUUCAAGAAAUAUGCGUGUAAUA |
| | AUGAAUAUUGGGAAACGUUUAAAGAAAACCCCAUCAGGCUUACUGAAGAAAACGUGGUAAAUUACA |
| | UUACCAAAUUAAAAGGACCAAAAGCUGCUGCUCUUUUUGCGAAGACACAUAAUUUGAAUAUGUUGC |
| | AGGACAUACCAAUGGACAGGUUUGUAAUGGACUUAAAGAGAGACGUGAAAGUGACUCCAGGAACAA |
| | AACAUACUGAAGAACGGCCCAAGGUACAGGUGAUCCAGGCUGCCGAUCCGCUAGCAACAGCGUAUC |
| | UGUGCGGAAUCCACCGAGAGCUGGUUAGGAGAUUAAAUGCGGUCCUGCUUCCGAACAUUCAUACAC |
| | UGUUUGAUAUGUCGGCUGAAGACUUUGACGCUAUUAUAGCCGAGCACUUCCAGCCUGGGGAUUGUG |
| | UUCUGGAAACUGACAUCGCGUCGUUUGAUAAAAGUGAGGACGACGCCAUGGCUCUGACCGCGUUAA |
| | UGAUUCUGGAAGACUUAGGUGUGGACGCAGAGCUGUUGACGCUGAUUGAGGCGGCUUUCGGCGAAA |
| | UUUCAUCAAUACAUUUGCCCACUAAAACUAAAUUUAAAUUCGGAGCCAUGAUGAAAUCUGGAAUGU |
| | UCCUCACACUGUUUGUGAACACAGUCAUUAACAUUGUAAUCGCAAGCAGAGUGUUGAGAGAACGGC |
| | UAACCGGAUCACCAUGUGCAGCAUUCAUUGGAGAUGACAAUAUCGUGAAAGGAGUCAAAUCGGACA |
| | AAUUAAUGGCAGACAGGUGCGCCACCUGGUUGAAUAUGGAAGUCAAGAUUAUAGAUGCUGUGGUGG |
| | GCGAGAAAGCGCCUUAUUUCUGUGGAGGGUUUAUUUUGUGUGACUCCGUGACCGGCACAGCGUGCC |
| | GUGUGGCAGACCCCCUAAAAAGGCUGUUUAAGCUUGGCAAACCUCUGGCAGCAGACGAUGAACAUG |
| | AUGAUGACAGGAGAAGGGCAUUGCAUGAAGAGUCAACACGCUGGAACCGAGUGGGUAUUCUUUCAG |
| | AGCUGUGCAAGGCAGUAGAAUCAAGGUAUGAAACCGUAGGAACUUCCAUCAUAGUUAUGGCCAUGA |
| | CUACUCUAGCUAGCAGUGUUAAAUCAUUCAGCUACCUGAGAGGGGCCCCUAUAACUCUCUACGGCU |
| | AACCUGAAUGGACUACGACAUAGUCUAGUCCGCCAAG |
| 34 | ATGGAAGATGCCAAAAACATTAAGAAGGGCCCAGCGCCATTCTACCCACTCGAAGACGGGACCGCC |
| | GGCGAGCAGCTGCACAAAGCCATGAAGCGCTACGCCCTGGTGCCCGGCACCATCGCCTTTACCGAC |
| | GCACATATCGAGGTGGACATTACCTACGCCGAGTACTTCGAGATGAGCGTTCGGCTGGCAGAAGCT |
| | ATGAAGCGCTATGGGCTGAATACAAACCATCGGATCGTGGTGTGCAGCGAGAATAGCTTGCAGTTC |
| | TTCATGCCCGTGTTGGGTGCCCTGTTCATCGGTGTGGCTGTGGCCCCAGCTAACGACATCTACAAC |
| | GAGCGCGAGCTGCTGAACAGCATGGGCATCAGCCAGCCCACCGTCGTATTCGTGAGCAAGAAAGGG |
| | CTGCAAAAGATCCTCAACGTGCAAAAGAAGCTACCGATCATACAAAAGATCATCATCATGGATAGC |
| | AAGACCGACTACCAGGGCTTCCAAAGCATGTACACCTTCGTGACTTCCCATTTGCCACCCGGCTTC |
| | AACGAGTACGACTTCGTGCCCGAGAGCTTCGACCGGGACAAAACCATCGCCCTGATCATGAACAGT |
| | AGTGGCAGTACCGGATTGCCCAAGGGCGTAGCCCTACCGCACCGCACCGCTTGTGTCCGATTCAGT |
| | CATGCCCGCGACCCCATCTTCGGCAACCAGATCATCCCCGACACCGCTATCCTCAGCGTGGTGCCA |
| | TTTCACCACGGCTTCGGCATGTTCACCACGCTGGGCTACTTGATCTGCGGCTTTCGGGTCGTGCTC |
| | ATGTACCGCTTCGAGGAGGAGCTATTCTTGCGCAGCTTGCAAGACTATAAGATTCAATCTGCCCTG |
| | CTGGTGCCCACACTATTTAGCTTCTTCGCTAAGAGCACTCTCATCGACAAGTACGACCTAAGCAAC |
| | TTGCACGAGATCGCCAGCGGCGGGGCGCCGCTCAGCAAGGAGGTAGGTGAGGCCGTGGCCAAACGC |
| | TTCCACCTACCAGGCATCCGCCAGGGCTACGGCCTGACAGAAACAACCAGCGCCATTCTGATCACC |
| | CCCGAAGGGGACGACAAGCCTGGCGCAGTAGGCAAGGTGGTGCCCTTCTTCGAGGCTAAGGTGGTG |
| | GACTTGGACACCGGTAAGACACTGGGTGTGAACCAGCGCGGCGAGCTGTGCGTCCGTGGCCCCATG |
| | ATCATGAGCGGCTACGTTAACAACCCCGAGGCTACAAACGCTCTCATCGACAAGGACGGCTGGCTG |
| | CACAGCGGCGACATCGCCTACTGGGACGAGGACGAGCACTTCTTCATCGTGGACCGGCTGAAAAGC |
| | CTGATCAAATACAAGGGCTACCAGGTAGCCCCAGCCGAACTGGAGAGCATCCTGCTGCAACACCCC |
| | AACATCTTCGACGCCGGGGTCGCCGGCCTGCCCGACGACGATGCCGGCGAGCTGCCCGCCGCAGTC |
| | GTCGTGCTGGAACACGGTAAAACCATGACCGAGAAGGAGATCGTGGACTATGTGGCCAGCCAGGTT |
| | ACAACCGCCAAGAAGCTGCGCGGTGGTGTTGTGTTCGTGGACGAGGTGCCTAAAGGACTGACCGGC |
| | AAGTTGGACGCCCGCAAGATCCGCGAGATTCTCATTAAGGCCAAGAAGGGCGGCAAGATCGCCGTG |
| | TAA |
| 35 | AUGGAAGAUGCCAAAAACAUUAAGAAGGGCCCAGCGCCAUUCUACCCACUCGAAGACGGGACCGCC |
| | GGCGAGCAGCUGCACAAAGCCAUGAAGCGCUACGCCCUGGUGCCCGGCACCAUCGCCUUUACCGAC |
| | GCACAUAUCGAGGUGGACAUUACCUACGCCGAGUACUUCGAGAUGAGCGUUCGGCUGGCAGAAGCU |
| | AUGAAGCGCUAUGGGCUGAAUACAAACCAUCGGAUCGUGGUGUGCAGCGAGAAUAGCUUGCAGUUC |
| | UUCAUGCCCGUGUUGGGUGCCCUGUUCAUCGGUGUGGCUGUGGCCCCAGCUAACGACAUCUACAAC |
| | GAGCGCGAGCUGCUGAACAGCAUGGGCAUCAGCCAGCCCACCGUCGUAUUCGUGAGCAAGAAAGGG |
| | CUGCAAAAGAUCCUCAACGUGCAAAAGAAGCUACCGAUCAUACAAAAGAUCAUCAUCAUGGAUAGC |
| | AAGACCGACUACCAGGGCUUCCAAAGCAUGUACACCUUCGUGACUUCCCAUUUGCCACCCGGCUUC |
| | AACGAGUACGACUUCGUGCCCGAGAGCUUCGACCGGGACAAAACCAUCGCCCUGAUCAUGAACAGU |
| | AGUGGCAGUACCGGAUUGCCCAAGGGCGUAGCCCUACCGCACCGCACCGCUUGUGUCCGAUUCAGU |
| | CAUGCCCGCGACCCCAUCUUCGGCAACCAGAUCAUCCCCGACACCGCUAUCCUCAGCGUGGUGCCA |
| | UUUCACCACGGCUUCGGCAUGUUCACCACGCUGGGCUACUUGAUCUGCGGCUUUCGGGUCGUGCUC |
| | AUGUACCGCUUCGAGGAGGAGCUAUUCUUGCGCAGCUUGCAAGACUAUAAGAUUCAAUCUGCCCUG |
| | CUGGUGCCCACACUAUUUAGCUUCUUCGCUAAGAGCACUCUCAUCGACAAGUACGACCUAAGCAAC |
| | UUGCACGAGAUCGCCAGCGGCGGGGCGCCGCUCAGCAAGGAGGUAGGUGAGGCCGUGGCCAAACGC |
| | UUCCACCUACCAGGCAUCCGCCAGGGCUACGGCCUGACAGAAACAACCAGCGCCAUUCUGAUCACC |
| | CCCGAAGGGGACGACAAGCCUGGCGCAGUAGGCAAGGUGGUGCCCUUCUUCGAGGCUAAGGUGGUG |
| | GACUUGGACACCGGUAAGACACUGGGUGUGAACCAGCGCGGCGAGCUGUGCGUCCGUGGCCCCAUG |
| | AUCAUGAGCGGCUACGUUAACAACCCCGAGGCUACAAACGCUCUCAUCGACAAGGACGGCUGGCUG |
| | CACAGCGGCGACAUCGCCUACUGGGACGAGGACGAGCACUUCUUCAUCGUGGACCGGCUGAAAAGC |
| | CUGAUCAAAUACAAGGGCUACCAGGUAGCCCCAGCCGAACUGGAGAGCAUCCUGCUGCAACACCCC |
| | AACAUCUUCGACGCCGGGGUCGCCGGCCUGCCCGACGACGAUGCCGGCGAGCUGCCCGCCGCAGUC |
| | GUCGUGCUGGAACACGGUAAAACCAUGACCGAGAAGGAGAUCGUGGACUAUGUGGCCAGCCAGGUU |
| | ACAACCGCCAAGAAGCUGCGCGGUGGUGUUGUGUUCGUGGACGAGGUGCCUAAAGGACUGACCGGC |
| | AAGUUGGACGCCCGCAAGAUCCGCGAGAUUCUCAUUAAGGCCAAGAAGGGCGGCAAGAUCGCCGUG |
| | UAA |
| 36 | ATGGAACTGCTGATCCTGAAGGCCAACGCCATCACCACCATCCTGACCGCCGTGACCTTCTGCTTC |
| | GCCAGCGGCCAGAACATCACCGAGGAATTCTACCAGAGCACCTGCAGCGCCGTGAGCAAGGGCTAC |
| | CTGAGCGCCCTGCGGACCGGCTGGTACACCAGCGTGATCACCATCGAGCTGTCCAACATCAAAGAA |
| | AACAAGTGCAACGGCACCGACGCCAAGGTGAAACTGATCAAGCAGGAACTGGACAAGTACAAGAAC |
| | GCCGTGACCGAGCTGCAGCTGCTGATGCAGAGCACCCCCGCCACCAACAACCGGGCCAGAAGAGAG |
| | CTGCCCCGGTTCATGAACTACACCCTGAACAACGCCAAGAAAACCAACGTGACCCTGAGCAAGAAG |
| | CGGAAGCGGCGGAGCGCCATCGCCAGCGGGGTGGCCGTGTCCAAGGTGCTGCACCTGGAAGGCGAG |
| | GTGAACAAGATCAAGTCCGCCCTGCTGTCCACCAACAAGGCCGTGGTGTCCCTGAGCAACGGCGTG |
| | AGCGTGCTGACCAGCAAGGTGCTGGATCTGAAGAACTACATCGACAAGCAGCTGCTGCCCATCGTG |
| | AACAAGCAGAGCTGCAGCATCAGCAACATCGAGACCGTGATCGAGTTCCAGCAGAAGAACAACCGG |
| | CTGCTGGAAATCACCCGGGAGTTCAGCGTGAACGCCGGCGTGACCACCCCCGTGAGCACCTACATG |
| | CTGACCAACAGCGAGCTGCTGTCCCTGATCAATGACATGCCCATCACCAACGACCAGAAAAAGCTG |
| | ATGAGCAACAACGTGCAGATCGTGCGGCAGCAGAGCTACTCCATCATGAGCATCATCAAAGAAGAG |
| | GTGCTGGCCTACGTGGTGCAGCTGCCCCTGTACGGCGTGATCGACACCCCCTGCTGGAAGCTGCAC |
| | ACCAGCCCCCTGTGCACCACCAACACCAAAGAGGGCAGCAACATCTGCCTGACCCGGACCGACCGG |
| | GGCTGGTACTGCGACAACGCCGGCAGCGTGAGCTTCTTCCCCCAAGCCGAGACCTGCAAGGTGCAG |
| | AGCAACCGGGTGTTCTGCGACACCATGAACAGCCTGACCCTGCCCTCCGAGGTGAACCTGTGCAAC |
| | GTGGACATCTTCAACCCCAAGTACGACTGCAAGATCATGACCTCCAAGACCGACGTGAGCAGCTCC |
| | GTGATCACCTCCCTGGGCGCCATCGTGAGCTGCTACGGCAAGACCAAGTGCACCGCCAGCAACAAG |
| | AACCGGGGCATCATCAAGACCTTCAGCAACGGCTGCGACTACGTGAGCAACAAGGGCGTGGACACC |
| | GTGAGCGTGGGCAACACACTGTACTACGTGAATAAGCAGGAAGGCAAGAGCCTGTACGTGAAGGGC |
| | GAGCCCATCATCAACTTCTACGACCCCCTGGTGTTCCCCAGCGACGAGTTCGACGCCAGCATCAGC |
| | CAGGTCAACGAGAAGATCAACCAGAGCCTGGCCTTCATCCGGAAGTCCGACGAGCTGCTGCACAAT |
| | GTGAATGCCGGCAAGAGCACCACCAATATCATGATCACCACAATCATCATCGTGATCATTGTGATC |
| | CTGCTGTCTCTGATTGCCGTGGGCCTGCTGCTGTACTGCAAGGCCCGCAGCACCCCTGTGACCCTG |
| | TCCAAGGACCAGCTGTCCGGCATCAACAATATCGCCTTCTCCAACTGA |
| 37 | AUGGAACUGCUGAUCCUGAAGGCCAACGCCAUCACCACCAUCCUGACCGCCGUGACCUUCUGCUUC |
| | GCCAGCGGCCAGAACAUCACCGAGGAAUUCUACCAGAGCACCUGCAGCGCCGUGAGCAAGGGCUAC |
| | CUGAGCGCCCUGCGGACCGGCUGGUACACCAGCGUGAUCACCAUCGAGCUGUCCAACAUCAAAGAA |
| | AACAAGUGCAACGGCACCGACGCCAAGGUGAAACUGAUCAAGCAGGAACUGGACAAGUACAAGAAC |
| | GCCGUGACCGAGCUGCAGCUGCUGAUGCAGAGCACCCCCGCCACCAACAACCGGGCCAGAAGAGAG |
| | CUGCCCCGGUUCAUGAACUACACCCUGAACAACGCCAAGAAAACCAACGUGACCCUGAGCAAGAAG |
| | CGGAAGCGGCGGAGCGCCAUCGCCAGCGGGGUGGCCGUGUCCAAGGUGCUGCACCUGGAAGGCGAG |
| | GUGAACAAGAUCAAGUCCGCCCUGCUGUCCACCAACAAGGCCGUGGUGUCCCUGAGCAACGGCGUG |
| | AGCGUGCUGACCAGCAAGGUGCUGGAUCUGAAGAACUACAUCGACAAGCAGCUGCUGCCCAUCGUG |
| | AACAAGCAGAGCUGCAGCAUCAGCAACAUCGAGACCGUGAUCGAGUUCCAGCAGAAGAACAACCGG |
| | CUGCUGGAAAUCACCCGGGAGUUCAGCGUGAACGCCGGCGUGACCACCCCCGUGAGCACCUACAUG |
| | CUGACCAACAGCGAGCUGCUGUCCCUGAUCAAUGACAUGCCCAUCACCAACGACCAGAAAAAGCUG |
| | AUGAGCAACAACGUGCAGAUCGUGCGGCAGCAGAGCUACUCCAUCAUGAGCAUCAUCAAAGAAGAG |
| | GUGCUGGCCUACGUGGUGCAGCUGCCCCUGUACGGCGUGAUCGACACCCCCUGCUGGAAGCUGCAC |
| | ACCAGCCCCCUGUGCACCACCAACACCAAAGAGGGCAGCAACAUCUGCCUGACCCGGACCGACCGG |
| | GGCUGGUACUGCGACAACGCCGGCAGCGUGAGCUUCUUCCCCCAAGCCGAGACCUGCAAGGUGCAG |
| | AGCAACCGGGUGUUCUGCGACACCAUGAACAGCCUGACCCUGCCCUCCGAGGUGAACCUGUGCAAC |
| | GUGGACAUCUUCAACCCCAAGUACGACUGCAAGAUCAUGACCUCCAAGACCGACGUGAGCAGCUCC |
| | GUGAUCACCUCCCUGGGCGCCAUCGUGAGCUGCUACGGCAAGACCAAGUGCACCGCCAGCAACAAG |
| | AACCGGGGCAUCAUCAAGACCUUCAGCAACGGCUGCGACUACGUGAGCAACAAGGGCGUGGACACC |
| | GUGAGCGUGGGCAACACACUGUACUACGUGAAUAAGCAGGAAGGCAAGAGCCUGUACGUGAAGGGC |
| | GAGCCCAUCAUCAACUUCUACGACCCCCUGGUGUUCCCCAGCGACGAGUUCGACGCCAGCAUCAGC |
| | CAGGUCAACGAGAAGAUCAACCAGAGCCUGGCCUUCAUCCGGAAGUCCGACGAGCUGCUGCACAAU |
| | GUGAAUGCCGGCAAGAGCACCACCAAUAUCAUGAUCACCACAAUCAUCAUCGUGAUCAUUGUGAUC |
| | CUGCUGUCUCUGAUUGCCGUGGGCCUGCUGCUGUACUGCAAGGCCCGCAGCACCCCUGUGACCCUG |
| | UCCAAGGACCAGCUGUCCGGCAUCAACAAUAUCGCCUUCUCCAACUGA |
| 38 | TTAAAACAGCTGTGGGTTGTTCCCACCCACAGGGCCCACTGGGCGCTAGCACTCTGATTTTACGAA |
| | ATCCTTGTGCGCCTGTTTTATATCCCTTCCCTAATTCGAAACGTAGAAGCAATGCGCACCACTGAT |
| | CAATAGTAGGCGTAACGCGCCAGTTACGTCATGATCAAGCATATCTGTTCCCCCGGACTGAGTATC |
| | AATAGACTGCTTACGCGGTTGAAGGAGAAAACGTTCGTTATCCGGCTAACTACTTCGAGAAGCCCA |
| | GTAACACCATGGAAGCTGCAGGGTGTTTCGCTCAGCACTTCCCCCGTGTAGATCAGGTCGATGAGC |
| | CACTGCAATCCCCACAGGTGACTGTGGCAGTGGCTGCGTTGGCGGCCTGCCTATGGGGAGACCCAT |
| | AGGACGCTCTAATGTGGACATGGTGCGAAGAGTCTATTGAGCTAGTTAGTAGTCCTCCGGCCCCTG |
| | AATGCGGCTAATCCTAACTGCGGAGCACATGCCTTCAACCCAGAGGGTAGTGTGTCGTAACGGGCA |
| | ACTCTGCAGCGGAACCGACTACTTTGGGTGTCCGTGTTTCTTTTTTATTCTTATATTGGCTGCTTA |
| | TGGTGACAATTACAGAATTGTTACCATATAGCTATTGGATTGGCCATCCGGTGTGTAATAGAGCTG |
| | TTATATACCTATTTGTTGGCTTTGTACCACTAACTTTAAAATCTATAACTACCCTCAACTTTATAT |
| | TAACCCTCAATACAGTTGACC |
| 39 | UUAAAACAGCUGUGGGUUGUUCCCACCCACAGGGCCCACUGGGCGCUAGCACUCUGAUUUUACGAA |
| | AUCCUUGUGCGCCUGUUUUAUAUCCCUUCCCUAAUUCGAAACGUAGAAGCAAUGCGCACCACUGAU |
| | CAAUAGUAGGCGUAACGCGCCAGUUACGUCAUGAUCAAGCAUAUCUGUUCCCCCGGACUGAGUAUC |
| | AAUAGACUGCUUACGCGGUUGAAGGAGAAAACGUUCGUUAUCCGGCUAACUACUUCGAGAAGCCCA |
| | GUAACACCAUGGAAGCUGCAGGGUGUUUCGCUCAGCACUUCCCCCGUGUAGAUCAGGUCGAUGAGC |
| | CACUGCAAUCCCCACAGGUGACUGUGGCAGUGGCUGCGUUGGCGGCCUGCCUAUGGGGAGACCCAU |
| | AGGACGCUCUAAUGUGGACAUGGUGCGAAGAGUCUAUUGAGCUAGUUAGUAGUCCUCCGGCCCCUG |
| | AAUGCGGCUAAUCCUAACUGCGGAGCACAUGCCUUCAACCCAGAGGGUAGUGUGUCGUAACGGGCA |
| | ACUCUGCAGCGGAACCGACUACUUUGGGUGUCCGUGUUUCUUUUUUAUUCUUAUAUUGGCUGCUUA |
| | UGGUGACAAUUACAGAAUUGUUACCAUAUAGCUAUUGGAUUGGCCAUCCGGUGUGUAAUAGAGCUG |
| | UUAUAUACCUAUUUGUUGGCUUUGUACCACUAACUUUAAAAUCUAUAACUACCCUCAACUUUAUAU |
| | UAACCCUCAAUACAGUUGACC |

TABLE 1A
SEQ ID NO Key

| SEQ | |
|---|---|
| ID | |
| NO: | Sequence |
| 1 | DNA encoding an empty TC83 self-replicating mRNA (5′ portion) |
| 2 | RNA encoding an empty TC83 self-replicating (5′ portion) |
| 3 | DNA sequence comprising a subgenomic promotor |
| 4 | RNA sequence comprising a subgenomic promotor |
| 5 | DNA encoding an empty TC83 self-replicating mRNA (3′ portion) |
| 6 | RNA encoding an empty TC83 self-replicating (3′ portion) |
| 7 | DNA encoding an empty TC83 self-replicating mRNA (5′ |
| | portion) comprising a NSP3 region comprising a G4796U |
| | nucleotide substitution and a G4944A nucleotide substitution |
| | (encoding an E1595D amino acid substitution and a |
| | V1645M amino acid substitution, respectively) |
| 8 | RNA encoding an empty TC83 self-replicating mRNA (5′ |
| | portion) comprising a NSP3 region comprising a G4796T |
| | nucleotide substitution and a G4944A nucleotide substitution |
| | (encoding an E1595D amino acid substitution and a |
| | V1645M amino acid substitution, respectively) |
| 9 | DNA encoding GFP |
| 10 | RNA encoding GFP |
| 11 | Amino acid sequence of GFP |
| 12 | DNA encoding GFP (duplicate) |
| 13 | RNA encoding GFP (duplicate) |
| 14 | Amino acid sequence of GFP (duplicate) |
| 15 | DNA encoding NS1 polypeptide |
| 16 | RNA encoding NS1 polypeptide |
| 17 | Amino acid sequence of NS1 polypeptide |
| 18 | DNA encoding PB1-F2 polypeptide |
| 19 | RNA encoding PB1-F2 polypeptide |
| 20 | Amino acid sequence of the PB1-F2 polypeptide |
| 21 | DNA encoding E3 polypeptide |
| 22 | RNA encoding E3 polypeptide |
| 23 | Amino acid sequence of E3 polypeptide |
| 24 | DNA encoding VP35 polypeptide |
| 25 | RNA encoding VP35 polypeptide |
| 26 | Amino acid sequence of VP35 polypeptide |
| 27 | RNA encoding N polypeptide |
| 28 | RNA encoding SOCS1 polypeptide |
| 29 | RNA encoding PIAS1 polypeptide |
| 30 | DNA encoding N polypeptide |
| 31 | Amino acid sequence of N polypeptide |
| 32 | DNA encoding an empty TC83 self-replicating mRNA (5′ |
| | portion) comprising a NSP2 region having an G3936C substitution |
| 33 | RNA encoding an empty TC83 self-replicating mRNA (5′ |
| | portion) comprising a NSP2 region having an G3936C substitution |
| 34 | DNA encoding FireFly Luciferase |
| 35 | RNA encoding FireFly Luciferase |
| 36 | DNA encoding respiratory syncytial virus prefusion F antigen |
| 37 | RNA encoding respiratory syncytial virus prefusion F antigen |
| 38 | DNA encoding EV71 internal ribosomal entry site (IRES) |
| 39 | RNA encoding EV71 IRES |

### General

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. The term “plurality” refers to two or more. Additionally, numerical limitations given with respect to concentrations or levels of a substance, such as solution component concentrations or ratios thereof, and reaction conditions such as temperatures, pressures and cycle times are intended to be approximate. The term “about” used herein is intended to mean the amount ±10%.

The invention will be further described by reference to the following, non-limiting, figures and examples.

### EXAMPLES

### Example 1. Project Summary

The present inventors initiated work on a synthetic, self-amplifying mRNA derived from the alphavirus genome, expressing interferon effector polypeptides of interest. The constructs are evaluated for protein expression and interferon expression.

### Methods

The self-replicating mRNAs and constructs of Table 2 were used in the Examples and comprised from 5′ to 3′ the following RNA sequences.

| Description of | 5′ End of self- | Construct | Subgenomic | Construct | 3′ End of self- |
|---|---|---|---|---|---|
| self- | replicating | coding region | promotor or | coding | replicating |
| replicating | mRNA (SEQ | (SEQ ID | IRES (SEQ ID | region(SEQ ID | mRNA (SEQ |
| mRNA | ID NO:) | NO:) | NO:) | NO:) | ID NO:) |
| GFP_nsP3 | 8 | 10 | — | — | 6 |
| mut | | | | | |
| GFP_NS1 | 2 | 10 | 4 | 16 | 6 |
| GFP_PB1-F2 | 2 | 10 | 4 | 19 | 6 |
| GFP_E3 | 2 | 10 | 4 | 22 | 6 |
| GFP_VP35 | 2 | 10 | 4 | 25 | 6 |
| GFP_N | 2 | 10 | 4 | 27 | 6 |
| GFP_SOCS1 | 2 | 10 | 4 | 28 | 6 |
| GFP_PIAS1 | 2 | 10 | 4 | 29 | 6 |
| Luc_GFP | Not provided | Not provided | Not provided | Not provided | Not provided |
| Luc_GFP | Not provided | Not provided | Not provided | Not provided | Not provided |
| DEAD | | | | | |
| GFP_nsP2 | 33 | 10 | — | — | 6 |
| mut | | | | | |
| Fluc_NS1 | 2 | 35 | 39 | 16 | 6 |
| Fluc_VP35 | 2 | 35 | 39 | 25 | 6 |
| Fluc_N | 2 | 35 | 39 | 27 | 6 |
| RSVF_NS1 | 2 | 37 | 39 | 16 | 6 |
| RSVF_VP35 | 2 | 37 | 39 | 25 | 6 |
| RSVF_N | 2 | 37 | 39 | 27 | 6 |

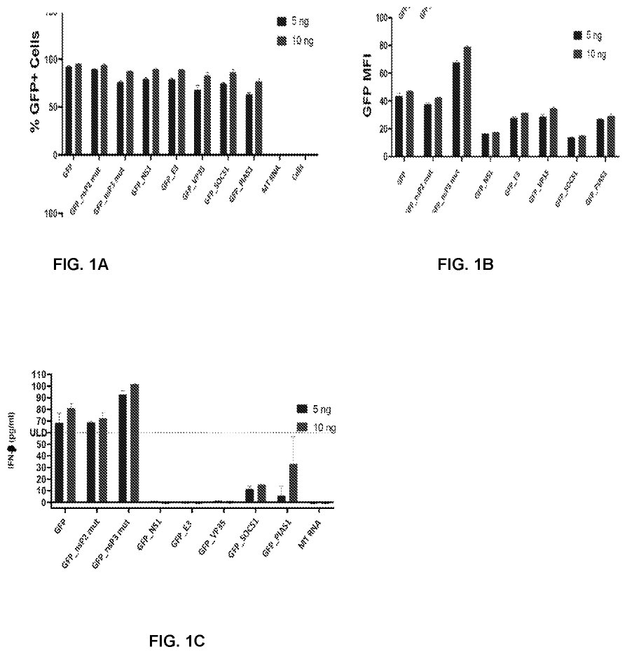

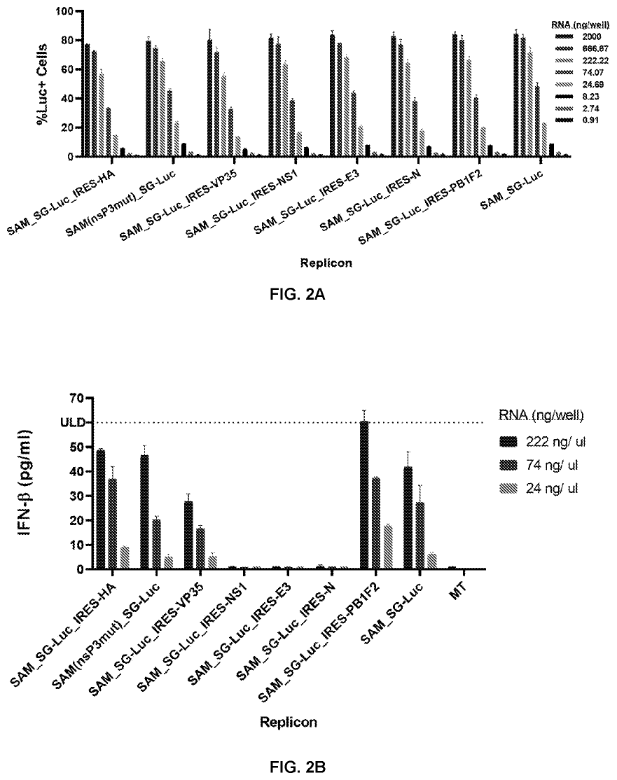

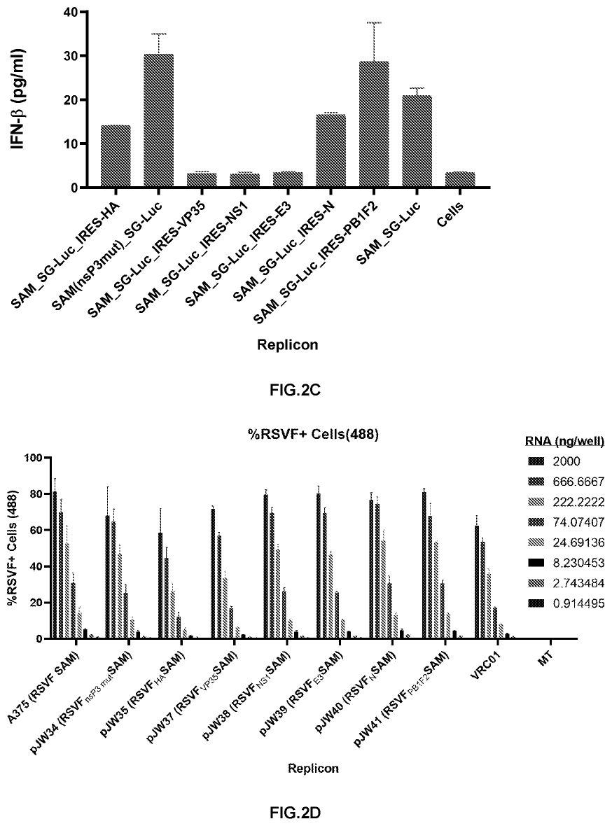

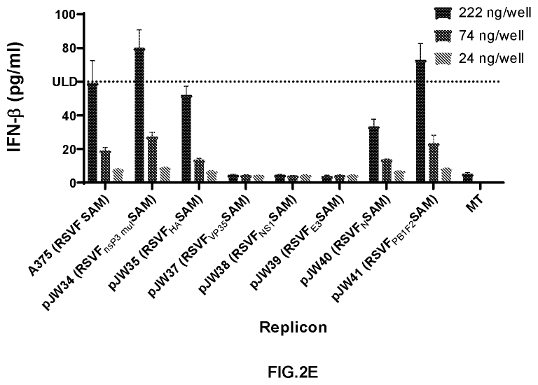

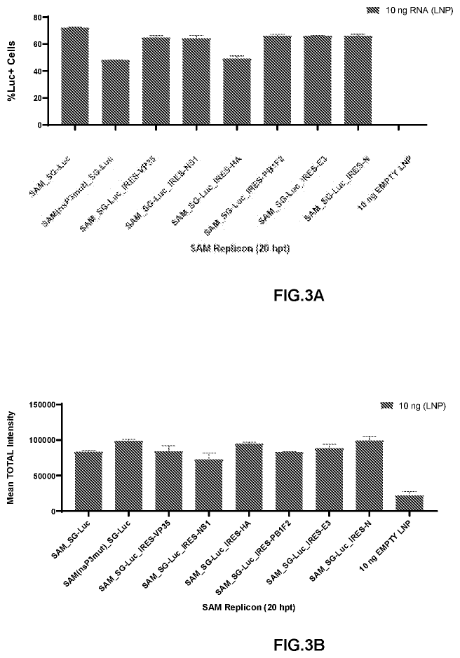

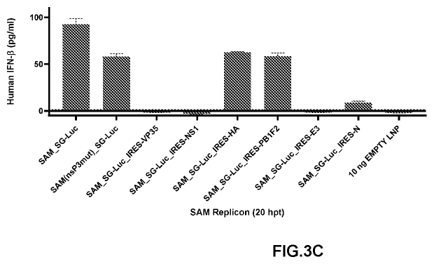

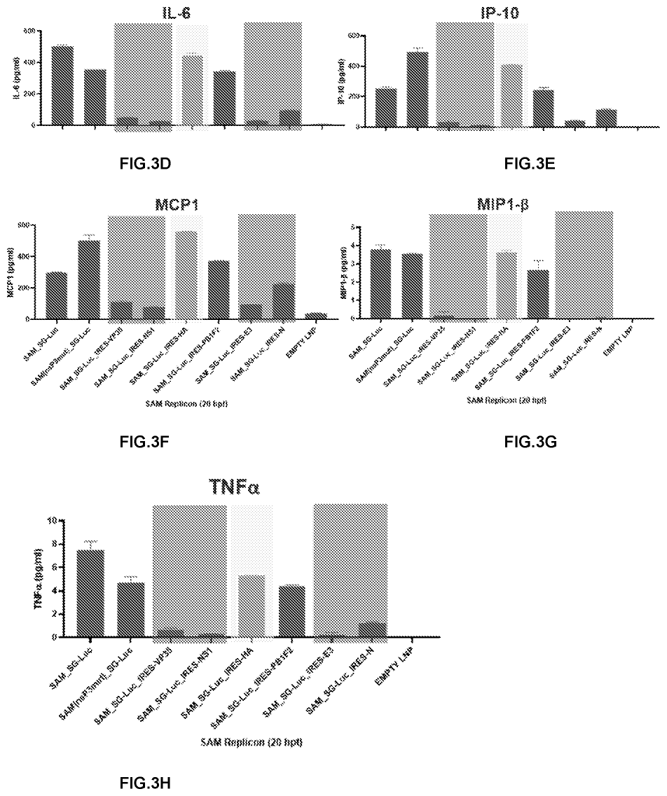

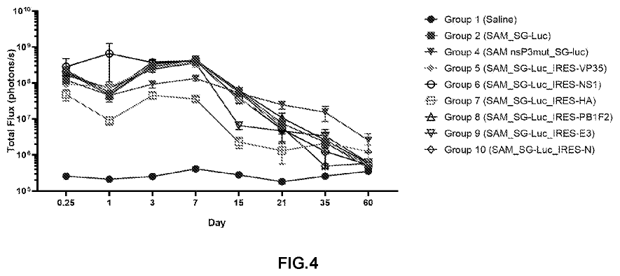

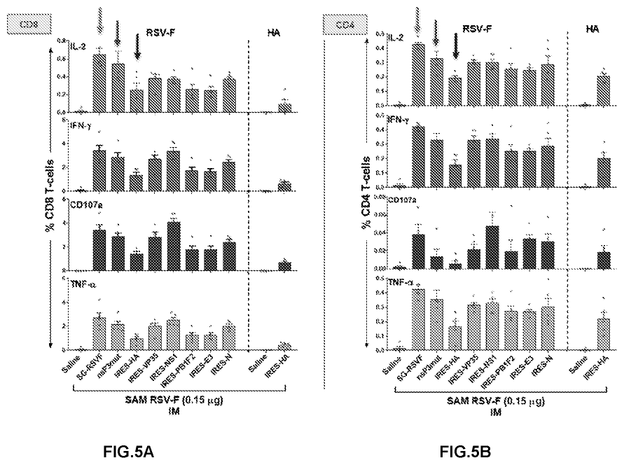

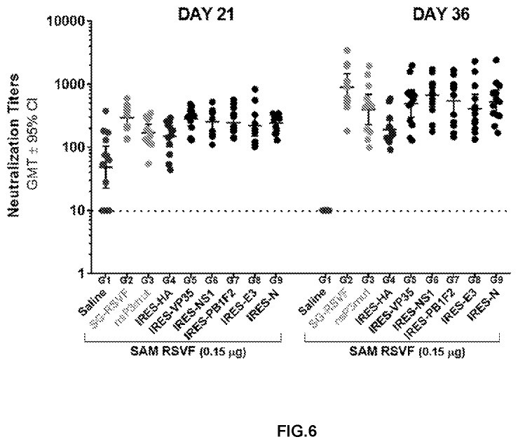

The SAM replicons were evaluated for potency in vitro in an immortalized mouse myoblast cell line via GFP expression reporter assays by flow cytometry, and secretion of IFN-β by ELISA, as readout for innate immune activation. Three SAM candidates interfaced uniquely with the IFN pathway, despite no change in the RNA potency. Interestingly, GFP SAM carrying mutations in the nsP3 region of the backbone (GFP_nsP3 mut) demonstrated consistently higher GFP mean fluorescent intensity (MFI), and consistently higher IFN induction, suggesting an IFN resistance phenotype. GFP-NS1 SAM (expressing and IFN effector from influenza) and GFP-VP35 SAM (expressing and IFN effector from EBOV), consistently demonstrated complete down regulation of IFN-β compared to the assay SAM controls, suggesting an IFN regulating phenotype. The results are summarized in FIG. 1A-FIG. 1C.

### Example 2: Viral Origin Interferon Effector Subset Study

### Plasmid DNA Construct Design and In Vitro RNA Transcription

To expand upon the results from “Example 1 Project Summary”, a subset of the interferon effector polypeptides of viral origin were cloned into plasmid DNA constructs similar to those discussed above with the following differences: (1) The model antigen firefly luciferase or the fusion protein of the Respiratory Syncytial Virus (RSV) were cloned into the plasmids downstream of the sub-genomic promoter following the encoded replicase machinery of VEEV(TC-83), (2) in this context the IFN modulating viral proteins were expressed under the control of the EV71 IRES which followed the stop codon of firefly luciferase or RSVF, respectively.

To produce in vitro transcribed RNA plasmid DNAs were linearized. Linearized DNA templates were purified by mixing then with equal volume of phenol: chloroform: isoamyl alcohol, followed by centrifugation. The aqueous phase was added to a clean eppendorf tube and 1:10 volume of 3M sodium acetate was added to each tube and 2:1 volume of 100% ethanol. Samples were chilled on ice for 20 minutes, and centrifuged for 30 minutes at 12,000 rpm. The supernatant was removed. The pellets were washed with 70% ethanol by centrifugation for 5 minutes. The supernatant was removed. The dried RNA pellets were resuspended in nuclease free water to the final DNA concentration of approximately 1 μg/μl.

T7 polymerase (NEB) was used for in vitro synthesis of the RNA and Vaccinia Capping Enzyme (NEB) for capping.

RNA was collected by centrifugation at 4200 rpm for 30 minutes at 4° C. The supernatant was removed and pellets were washed by adding 5 ml 70% ethanol and centrifugation for 5 min., 4200 rpm, at 4° C. The RNA pellets was air dried for 1 minute. The RNA was dissolved in nuclease-free water and the RNA concentration was measured by using a NanoDrop spectrophotometer (Thermo Fisher Scientific). The RNA quality was assessed by agarose gel electrophoresis.

### Potency and Analysis of IFN-β Expression in Mouse Myoblast Cells (C2C12)

To determine if the firefly luciferase and RSVF versions of the IFN modulating SAM replicons expressed the respective antigens and behaved similarly with respect to innate IFN signaling a potency test was performed by evaluating the percent of cells in a transfected population expressing the antigens by flow cytometry and the supernatants from these transfected cells were analyzed for IFN-β expression by ELISA. C2C12 cells were plated at 1e7 cells in T225 flasks in Growth Media (DMEM+5% fetal bovine serum (FBS)+penicillin/streptomycin/glutamate (PSG), and Incubated at 37° C., 5% C02 for −24 hours. 100 ul outgrowth media (DMEM+1% FBS+P/S/G) was added to each well of a 96-well flat bottom plates, and warmed to 37° C. RNA dilutions were prepared for electroporation resulting in the 1:3 dilutions of the experimental RNA ranging from 2,000 ng down to 0.91 ng. Each electroporation was supplemented with mouse thymus RNA such that each electroporation contained a total RNA concentration of 2,000 ng. Growth phase cells were harvested by washing with PBS and trypsinizing in 0.25% trypsin-EDTA for 5 min. The cells were washed with ice cold Opti-MEM media, and resuspended to a concentration of 2e5 cells/100 ul Opti-MEM media. 100 ul of cell mixture containing 2e5 cells per electroporation was added to 10 ul aliquots containing the specific RNA mixtures. Cell and RNA mixtures were electroporated with one 25 ms pulse at 120 V (2 mm gap). The electroporated cells were allowed to rest at room temperature for 10 mins. Cells were transferred from the electroporation plate to pre-warmed 96 well flat bottom cell culture plate mentioned above, and cells were incubated for 18 hours at 37° C., 5% C02.

The following day, duplicate supernatants (˜150 ul each) were collected with multi-channel pipette for each construct, and placed into 96-well round bottom plate. The plate was spun at 4° C., 1500 rpm, 5 min, to clarify the supernatant. Supernatants were transferred to new round bottom 96-well plates, sealed, and placed at −80° C. until ELISA analysis (see below).

To prepare the cells for flow cytometric analysis after removing the supernatants, the cells were trypsinized with 100 ul 0.25% Trypsin-EDTA, and incubated at 37° C. for 5 min. 100 ul DMEM+5% FBS cell culture media was added to each well to terminate the trypsin reactions. Cells were mixed and transferred to a new round bottom 96 well dish. The cells were spun at 1500 RPM, 5 min, 4° C. The supernatants were removed and the cells were resuspended in 200 ul/well Cytofix/cytoperm (BD) and incubated at 4° C. for 30 minutes. The cells were spun down as above and washed 2× with 0.2 ml/well Perm-Wash Buffer. The cells were then resuspended in 100 ul Perm-wash buffer containing 1:1000 anti-Fluc or anti-RSVF antibody, and incubated for 1 hr at 21° C. The cells were washed 2× with 0.2 ml/well Perm-Wash Buffer and resuspended in 100 ul Perm-Wash buffer containing 1:1000 anti-human or anti-mouse Alexa Fluor 488, respectively, and incubated 30 min at 21° C., in the dark. The cells were washed 2× with 200 ul FACS buffer, and resuspended in 200 ul FACS buffer. The cell were analysed the same day on the MacsQuant VYB flow cytometer, and the potency was measured (% positive Luciferase or RSVF cells, respectively).

The supernatants harvested above were analyzed for IFN-β expression for both the Firefly Luciferase and RSVF IFN modulating SAM constructs using VeriKine-HS Mouse IFN Beta Serum ELISA Kits (pbl Assay Science #42410) to determine if these new constructs performed similarly to the previously described GFP constructs in terms of the down regulation of IFN-β in the supernatants from C2C12 transfected cells. The ELISAs were performed as per the manufacturer's instructions. In brief, the standards provided in the kits were diluted in DMEM+1% FBS resulting in standards ranging from 60 pg/ml down to 0.94 pg/ml. The supernatant samples representing the ˜222, ˜74, and ˜24 ng/well RNA transfections were diluted 1:5 prior to addition of the sample diluent provided in the ELISA kit. 50 ul Sample Diluent Buffer was added to each well of 96-well ELISA plate. 50 ul of the sample supernatants, in duplicate, were added to 50 ul of Sample Diluent Buffer in the 96-well plate. Incubated, shaking at 21° C. (1 hr, 650 rpm). Media was aspirated off, and the wells were washed 4× with provided wash buffer. 50 ul diluted antibody solution was added to each well of 96-well ELISA plate. Incubated, shaking at 21° C. (30 min, 650 rpm). Aspirate and wash as above. Added 50 ul Diluted HRP solution to each well. Incubated, shaking at 21° C. (10 min, 650 rpm). Aspirate and wash as above. Added 100 ul TMB Substrate. Incubated at 21° C. (10 min, in dark, no shaking). Read plate on the Glomax at 450 nm.

It was hypothesized that LNP formulated IVT RNA expressing the IFN modulating SAM replicons would down regulate IFN-β similar to the electroporated IVT RNA. To test this C2C12 cells were plated at 1e5 cells/well in culture media (DMEM+10% FBS+1% Penicillin and Streptomycin) a 96-well plate, and were incubated at 37° C. for 4 hr. 100 ul of media containing 10 ng of each RV39 LNP formulated SAM Luciferase constructs were added to each well in triplicate. Supernatants were collected at 16 hr post-transfection. Samples were diluted 1:8 prior to analysis by ELISA, as described above.

### Results

The C2C12 potency analysis revealed that when compared to control SAM replicons expressing luciferase alone, or RSVF alone, as well as to a control SAM expressing the innate immune-inert influenza HA protein after the respective antigens, the SAM IFN modulating replicons expressing the corresponding antigens were similarly potent across multiple RNA concentrations in mouse myoblast cells (as measured by percent of cells expressing the respective antigen, by flow cytometric analysis. FIGS. 2A and 2D, respectively).

Analysis of the IFN-β expression levels by ELISA showed that SAM IFN modulating replicons down regulated IFN-β expression when the viral proteins were expressed in the context of wither the luciferase or RSVF constructs when compared to the antigen alone and antigen-HA controls (FIGS. 2B and 2E, respectively). The VP35, NS1, E3, and N protein constructs all substantially decreased IFN-β levels in the C2C12 cells supernatant. This data correlated well with the IFN-β ELISA data produced from the LNP formulated GFP constructs as shown in FIG. 1C (Note: the N protein was not analyzed in the LNP formulated GFP construct context.

It was determined that the formulated luciferase versions of the SAM IFN modulating replicons behaved similarly in C2C12 cells to the unformulated/electroporated IVT RNA, with the VP35, E3, and NS1 constructs substantially decreasing IFN-β expression. While the N protein construct did decrease IFN-β expression in this context, it was not as robust as the other constructs (FIG. 2C).

### Example 3. Human Skeletal Muscle Primary Cell Transfections with IFN Modulating SAM Replicons for Potency (HCl), and Innate Signaling Analysis (Luminex, NanoString and ELISA-Human IFN-β)

To evaluate whether the results obtained in the mouse myoblast cells (C2C12) were repeatable in human cells the IFN modulating SAM replicons expressing firefly luciferase were tested in primary human skeletal muscle cells (HSKM). HSKM cells were rinsed with sterile PBS and trypsinized with 3 ml warm trypsin for 5 min at 37° C. The trypsin was neutralized and the cells were mixed gently and counted. The cells were spun down at 3000 rpm for 3 minutes at 22° C., and resuspended in 10 ml growth media. Cells were dilute to 3e5 cells/ml in growth media. Added 100 ul growth media to each well of 96 well plate. Added 100 ul of diluted cells (approximately 15-30,000 cells) per well (96 well plate), and incubated overnight ˜20 hours. The growth media was removed and 100 ul perwell growth media (containing FBS) was added. Incubated for 1 hr. LNP dilutions were prepared. Added 100 ul of appropriate dilution to each well of 96 well plate and incubate overnight (for ˜12 or ˜20 hrs). The cells were dosed with either 10 ng LNP/100 ul or 1 ng/100 ul in 3 ml total media. Note, replicated plates were transfected to accommodate the downstream analyses.

The following day the cells were fixed and stained to determine potency by analysis on a CX7 High Content Imager (HCl). Removed the 200 ul media/supe from the cells (saved for ELISA/Luminex analysis in 96-well round-bottomed plate, cover and place at −80C until use). Added 100 ul 4% PFA/PBS and incubated 30 min at 21° C. The cells were washed with 200 ul PBS and 75 ul 0.1% Triton X-100 was added, with incubation for 10 min at 21° C.

The cells were washed with 200 ul PBS 2×. Added 100 ul 1:1000 Primary antibody diluted in PBS to each well (Mouse Monoclonal Anti-Firefly Luciferase [Luci17](ab16466, abcam)). Incubated 60 min at 32° C. Cells were washed with 200 ul PBS 3×. Added 100 ul 1:1000 Secondary antibody+1:2000 dilution of DAPI to each well (Goat anti-Mouse IgG (H+L) Highly Cross-Adsorbed Secondary Antibody, Alexa Fluor 488 (ThermoFisher A11029)) (DAPI (Thermofisher—62247)). Incubated 60 min., in the dark at 21° C. The cell were washed with 200 ul PBS 3×. The final wash was left on and the cells were covered with seal and foil and stored at 4° C. until scanning on CX7 HCl.

In order to evaluate IFNβ induction by ELISA in supernatants from HSKM cells that were expressing IFN effector SAM replicons with Luciferase “antigen” we performed an ELISA experiments using the following ELISA Kit: VeriKine-HS Human IFN Beta Serum ELISA Kit (pbl Assay Science #41415). The ELISA was performed as per the manufacturer's instructions (PROTOCOL A, as denoted by the manufacturer and summarize below): The kit standard curve was setup in HSKM cell Media, producing standards ranging from 150 pg/ml down to 1.2 pg/ml. The 12 hr post-transfection samples were undiluted (1 ng LNP dose) or diluted 1:2 (10 ng LNP dose) while the 20 hr post-transfection samples were diluted 1:2 (1 ng LNP dose) or diluted 1:10 (10 ng LNP dose). Dilution of the supernatant samples occurred prior to addition of Antibody/Assay diluent provided in the kit.

To prepare supernatants for Luminex protein cytokine/chemokine evaluation, supernatants collected from the 12 and 20 hr HSKM cell timepoints above we processed. Followed the manufacturer's protocol for the Luminex kit (described below), with a minor modification of the initial spin of supernatants to 4100 Xg 10 min from provided protocol. The relevant analytes are listed below for the Human Cytokine/Chemokine/Growth Factor Panel A Magnetic Bead Panel MILLIPLEX ASSAY KIT (Millipore Sigma, HCYTA-60K-17): IL-1α, IL-1β, IL-2, IL-4, IL-6, IL-8, IL-10, IL-12 (p40), IL-12 (p70), TNFα, IL-18, IFNα2, IFNγ, IP-10, GM-CSF, MIP-1p, MCP-1. The raw luminex data was processed and analyzed using Belysa (Mlllipore Sigma software), and graphed using Graphpad Prism.

To investigate the changes in the inflammatory gene transcriptomic profile following SAM IFN modulator transfection in HSKM cells modulators transcriptome analysis was performed on the above transfected HSKM cells using Nanostring Human Inflammation kit. In brief, to generate cell lysates fir this experiment, media was removed from the wells at 20 hr post-transfection and washed with PBS. Added 20 ul of the 30% Buffer RLT (QIAGEN, PN: 79216)/1:300 BME mix to each well, scraped the cells with pipet tip, and transfered to a PCR tube 96-well plate, and stored at −80C until further use. Analysis was then performed on the Nanostring instument with the following kits: XT_PGX_HuV2_Inflammation_CSO (Nanostring PN: 115000072), and nCounter Master Kit—48 rxns NAA-AKIT-048 (Nanosting PN: 100054). The samples were processed as per the manufacturer's instructions: (MAN-10056-02 “CodeSet Hybridization Setup”). 0.84 ul of 50 mg/ul Proteinase K (ThermoFisher) were added to the CodeSet Master Mix. The samples were immobilized on the provided cartridge following the vendors instructions: (MAN-00035_nCounter_Analysis_System_MAX_FLEX, pages: 16-27). Readings were then taken on the nCounter reader with the following settings: 555 fields, RLF files provided by vendor: NS_Infl_Hs_v2_C2534.rlf

### Results

The HCl potency results demonstrated that, as in the C2C12 mouse myoblast cells, formulated IFN modulating SAM replicons expressing the luciferase antigen were similarly potent between contracts in HSKM cells, as measured by percent cells expressing the constructs and by the mean total intensity of the cells in each well of the 96-well plate (FIGS. 3A and 3B).

Intriguingly, the VP35, NS1, N, and E3 constructs all performed as expected, substantially down regulating IFN-β as measured by ELISA in the supernatants of the HSKM cells, when compared to the luciferase alone, and luciferase-HA control contracts (FIG. 3C). This data not only correlated with the luciferase contract data from C2C12 cells, but it also correlated with the data from the original GFP versions of the constructs in C2C12 cells (FIG. 1C).

Analysis of the Luminex Human Cytokine/Chemokine Magnetic Bead Panel revealed that the down regulation of innate signaling factor by the IFN modulating SAM replicons went beyond IFN-β readout at the protein level, and several inflammatory factors from the panel tested were substation ally downregulated in the supernatants of HSKM cell when compared to the luciferase alone and luciferase-HA constructs, including; IL-6, IP-10, MCP-1, MIP1-β and TNFα (FIG. 3D-H). The VP35, and NS1 replicons consistently and substantially reduced the levels of the aforementioned analytes, while the E3 and N protein replicons consistently down regulated these analytes, though to a lesser extent across the panel.

The transcriptomic Nanostring analysis revealed that the SAM constructs expressing IFN modulators (VP35, NS1, E3, N) had a distinct global transcriptomic profile compared to the SAM replicons expressing luciferase alone or luciferase-HA, corroborating the Luminex cytokine/chemokine analysis, as well asl the ELISA data described above (Data not shown).

Herein, it has been demonstrated that in HSKM cells it is possible to modulate the innate cytokine activation profile at both the transcriptomic, and protein level, using viral protein expression driven by SAM mRNA technology.

### Example 4. In Vivo Studies: Evaluation of the Effects of IFN Modulating SAM Replicons on Antigen Expression and Immunogenicity in Mice

To evaluate the SAM candidates' phenotype in vivo, specifically their putative effects on antigen expression, the RV39 LNP formulated SAM interferon regulating candidates expressing firefly luciferase were tested in an in vivo mouse experiment (Balb/c mice) by intramuscular injection (0.15 ug LNP formulated SAM/mouse), along with their relevant controls, to determine their effects on innate immune responses, as measured by cytokines released in the sera at 6h, 24h, 21 and 60 days (data not shown), and antigen expression, as measured by bioluminescence at different time points after administration using an IVIS bioimaging system (PerkinElmer). As controls, additional SAM vectors encoding FLuc and IFN regulating proteins having milder or no effect were tested in the same manner.

To evaluate the SAM candidates' phenotype in vivo, specifically their putative effects on immunogenicity, the RV39 LNP formulated SAM interferon regulating candidates expressing RSV-F were tested in an in vivo mouse experiment (Balb/c mice) by intramuscular injection (0.15 ug LNP formulated SAM/mouse), along with their relevant controls, to determine their effects on innate and adaptive immune responses, as measured by cytokines released in the sera (data not shown), intracellular cytokine staining (ICS) for T cell responses at different time points after administration, and RSV neutralizing antibody quantification. As controls, additional SAM vectors encoding RSV-F and IFN regulating proteins having milder or no effect will be tested in the same manner.

To evaluate T-cell responses splenocytes were collected from 5 mice per group at 2wp2, and RSVF-specific T cell responses were assessed by intracellular cytokine staining and multi-parametric flow cytometry. Briefly, single cell suspensions of 1-2×10⁶ live splenocytes were plated in 96-well U-bottom plates and incubated overnight at 37° C. with RSV-F specific, or influenza HA specific peptide pools. Golgi transport inhibitor, BFA, was added and the splenocytes incubated for an additional 4 hours at 37° C. Cells were then stained with a LIVE/DEAD stain (Invitrogen) for 20 minutes at 21° C. Cells were subsequently washed, fixed, and permeabilized with Cytofix/Cytoperm (BD Biosciences) for 20 minutes at 4° C., blocked with diluted mouse Fc Block (anti-CD16/CD32) in 1×Perm/Wash Buffer (BD Biosciences) for 10 minutes at 21° C., and stained with a mouse Intracellular Cytokine Panel (ICS) including; CD3, CD4, CD8, CD44, IFNγ, IL2, TNFα, IL13, IL4, and IL17F. Finally, the cells were washed and suspended in PBS plus 1% BSA (Gibco, Thermo Scientific). Data was acquired on the BD Symphony (Mozart) using the High Throughput Sampler (HTS) and data were analyzed using FlowJo v10 software (BD Biosciences).

To evaluate RSV neutralizing antibody quantification a plaque reduction assay was performed in a 96-well format. Its purpose was to detect and quantify neutralizing antibodies to the Respiratory Syncytial Virus (RSV) subtype A Long strain raised in mice in response to vaccination that can inhibit the virus ability to infect cells and generate syncytia. Vero cells are seeded in 96 well plates at a final concentration of 1.6×10⁴ cells/well and are incubated overnight (O/N) at 37° C., 5% C02. Heat inactivated experimental and reference serum sample (heat inactivated cotton rat antiserum to RSV from Sigmovir) dilutions and virus-serum mixtures are prepared in 96-well round-bottom plates, and then transferred to the seeded cells in 96-well flat-bottom plates. Sera-virus mixtures are incubated for 2 h at 35° C., 5% C02 and transferred into the previously seeded flat bottom 96-well plates. Plates are them incubated for 2 h at 35° C., 5% C02. The sera-virus mixtures are removed and 200 uL 0.5%-CMC/RSV media was added to all wells. The plates are incubated for 42-48 h at 35° C., 5% C02. The plates were developed with anti-RSV, and staining with TrueBlue substrate. Media was removed and 100 uL/well of 10% neutral formalin solution was added. The plates were incubated for 60 min at 21° C. The formalin was removed and discarded. 100 ul/well of block buffer (0.5% Saponin/3.0% FBS) was added to the wells and incubated for 1 h at 21° C. Mouse anti-RSV Fusion Protein monoclonal antibody and Mouse anti-RSV Nucleoprotein were dilute 1:1000 block buffer (0.5% Saponin), and 100 uL/well was added to the plates. The plates were incubated for 1 h at 21° C. The plates were washed 3× with 300 uL/well PBST (1×PBS and 0.5% Tween-20) using a plate washer. Anti-Goat IgG-HRP was dilute 1:1000 in block buffer (0.5% Saponin) and 100 uL/well was added to the plates, and incubated for 1 h at 21° C. The plates were Washed as above. 100 uL/well of TrueBlue substrate (KPL) was added to each well and incubated 15 min at 21° C. (in the dark). The plates were washed 2× with 300 uL/well dH₂O and air dried in the dark. The plates were scanned using Immunospot 5.0 Analyzer Pro DC software to scan plate(s) on a CTL ELISpot Reader and plaques were counted using Biospot 5.0 Professional software. The serum dilutions versus the percent of plaque reduction obtained were plotted, comparing the number of the plaques in the serum dilution wells from the serum sample to the number of plaques in wells infected with RSV virus alone (100%). The assay results were expressed as 60% neutralization titers (ED60). Plaque reduction titers are calculated by regression analysis of the inverse dilution of serum that provided a 60% plaque reduction compared to control wells incubated without serum. The titers are calculated considering final dilution of serum on cells rather than serum starting dilution.

### Results

When evaluating the in vivo bioluminescence data presented here it is important to understand that when multiple proteins are being expressed from a SAM replicon after the sub-genomic promoter the total expression of the primary antigen, in this case firefly luciferase is reduced. This analysis revealed this trend, with the luciferase-HA control SAM construct expressing ˜1 log less total flux over the course of the study as compared to the luciferase alone control construct. The VP35, NS1, E3, and N protein SAM constructs all increase the total flux to levels comparable to the luciferase alone construct, suggesting that the IFN modulating proteins being expressed may increase the total antigen expression levels across the timeline of the experiment. It is hypothesized that this increase is likely due to the down regulation of the innate immune response, as suggested by the in vitro data described above (FIG. 4). Though luminex evaluation was performed on the sera from the mice in this study to evaluate sera cytokine levels, similar to the supernatant analysis performed and described above for the HSKM cells, the study was under-dosed for such a readout, as the levels of sera cytokine detected were barely above the lower limit of quantitation (LLOQ) for the assay, and were at the lower end of the capacity these kits are capable of detecting, so no trends in sera cytokine reduction were observed (data not shown).

The CD8 and CD4 T cell mediated immunity analysis from the spleens of mice vaccinated with SAM IFN modulating replicons expressing RSVF revealed that mice vaccinated with the VP35, NS1, and N SAM constructs had T cell activation levels notably higher that the RSVF-HA construct, and which were closer to the levels of activation achieved by vaccination with the RSVF alone SAM construct (FIGS. 5A and 5B). This trend correlates with the antigen expression bioluminescence analysis above, and corroborated the hypothesis that decrease innate signaling may lead to increased antigen expression, possible resulting in a more robust adaptive immune response.

This trend was observed again when the analysis of neutralizing antibodies to RSVF was completed, demonstrating that mice vaccinated with the IFN modulating SAM constructs had notably higher Nab titers when compared to the RSVF alone and RSVF-HA control constructs (FIG. 6A). When taken together, in vivo bioluminescence, neutralizing antibody level, and T-cell activation profile data suggest that these IFN modulating SAM replicons may be modulating immune responses to the RSV fusion protein in vivo, and potential increasing antigen expression. This work offers novel proof-of-concept data demonstrating the ability to tune innate immune signalling in an effort to combat reactogenicity from the SAM vaccine platform.

1. A composition comprising a self-replicating messenger ribonucleic acid (mRNA) comprising a construct encoding a first heterologous polypeptide interferon effector that suppresses an interferon response, wherein the heterologous polypeptide interferon effector is
(a) VP35, or a variant or fragment thereof; or
(b) N, or a variant or fragment thereof;
wherein the self-replicating mRNA further comprises a construct encoding a polypeptide selected from the group consisting of: a polypeptide antigen; an antigen-binding polypeptide; an immune-modulatory polypeptide; or a therapeutic polypeptide.

2. The composition of claim 1, wherein the construct encodes a second heterologous polypeptide interferon effector that suppresses an interferon response, wherein the second heterologous polypeptide interferon effector is different from the first and is selected from the group consisting of:
(a) VP35, or a variant or fragment thereof;
(b) N, or a variant or fragment thereof;
(c) NS1, or a variant or fragment thereof; and
(d) E3, or a variant or fragment thereof.

3. A composition comprising a self-replicating mRNA comprising a construct encoding a heterologous polypeptide interferon effector that enhances an interferon response, wherein the heterologous polypeptide interferon effector is PB1-F2, or a variant or fragment thereof, wherein the self-replicating mRNA further comprises a construct encoding a polypeptide selected from the group consisting of: a polypeptide antigen; an antigen-binding polypeptide; an immune-modulatory polypeptide; or a therapeutic polypeptide.

4. The composition of claim 1, wherein the self-replicating mRNA comprises modified sequence that modulates the self-replicating mRNA response to interferon.

5. The composition of claim 4, wherein the self-replicating mRNA comprises a NSP3 region and the modified sequence comprises an amino acid substitution within the NSP3 region.

6. The composition of claim 5, wherein the NSP3 region is a TC83 NSP3 region that encodes an E1595D amino acid substitution, a V1645M amino acid substitution, or both.

7. The composition of claim 6, wherein the RNA sequence encoding the amino acid substitution within the NSP3 region comprises a G4796U nucleotide substitution, a G4944A nucleotide substitution, or both.

8. (canceled)

9. The composition of claim 1, wherein the construct encodes two or more polypeptides selected from the group.

10. (canceled)

11. (canceled)

12. (canceled)

13. The composition of claim 1, wherein the the construct comprises one or more polynucleotide sequences encoding a polypeptide selected from the group consisting of (i) a polypeptide comprising the sequence of SEQ ID NO:17, SEQ ID NO:23; or SEQ ID NO:26, (ii) a polypeptide having at least 90% identity to SEQ ID NO:17, SEQ ID NO:23, or SEQ ID NO:26, or (iii) a polypeptide comprising a fragment of SEQ ID NO:17, SEQ ID NO:23, or SEQ ID NO:26 that is up to 10 amino acids shorter than full-length sequence.

14. The composition of claim 3, wherein the construct (b) comprises one or more polynucleotide sequences encoding a polypeptide selected from the group consisting of (i) a polypeptide comprising the sequence of SEQ ID NO:20; (ii) a polypeptide having at least 90% identity to SEQ ID NO:20, or (iii) a polypeptide comprising a fragment of SEQ ID NO:20 that is up to 10 amino acids shorter than full-length sequence.

15. (canceled)

16. The composition of claim 5, wherein the self-replicating mRNA comprises
a polynucleotide sequence comprising (i) the sequence of SEQ ID NO:8, (ii) a polynucleotide having at least 90% identity to SEQ ID NO:8, or (iii) a fragment of SEQ ID NO:8 that is up to 30 nucleic acids shorter than full-length sequence.

17. The composition of claim 1 wherein the self-replicating mRNA (i) comprises a capy 1 and (ii) is encapsulated in a liposome.

18. The composition of claim 3, wherein the self-replicating mRNA (i) comprises a capy 1 and (ii) is encapsulated in a liposome.

19. The composition of claim 1, wherein the self-replicating mRNA comprises at least one N1-methylpseudouridines (N1Ψ).

20. The composition according to claim 3, wherein the self-replicating mRNA comprises at least one N1-methylpseudouridines (N1Ψ).

21. A DNA molecule encoding the self-replicating RNA of claim 1.

22. A DNA molecule encoding the self-replicating RNA of claim 3.

23. A process for producing a formulated interferon effecting or modulating self-replicating mRNA comprising a step of transcribing the DNA molecule of claim 21 in vitro to produce a self-replicating mRNA molecule and a step of formulating the self-replicating mRNA encapsulated in a liposome.

24. A process for producing a formulated interferon effecting or modulating self-replicating mRNA comprising a step of transcribing the DNA molecule of claim 22 in vitro to produce a self-replicating mRNA molecule and a step of formulating the self-replicating mRNA encapsulated in a liposome.

25. (canceled)

26. (canceled)

27. (canceled)

28. (canceled)

29. (canceled)

30. A method of effecting or modulating an interferon response in a subject in need thereof, comprising administering the composition of claim 1 to the subject.

31. A method of inducing an interferon response in a subject in need thereof, comprising administering the composition of claim 3 to the subject.

32. The method of claim 30, wherein the subject is human.

33. The method of claim 3, wherein the subject is human.

34. (canceled)

35. A composition comprising a liposome comprising a mRNA encoding VP35, or a variant or fragment thereof.

36. The composition of claim 35, wherein the mRNA further comprises sequence recognized by a replicase encoded by a self-replicating mRNA, such that the mRNA can be amplified in trans by in the presence of the replicase.

37. (canceled)

38. (canceled)
